SEQUENCE LISTING (24 sequences)

SEQ ID NO: 1 - DNA, 33 bp, Artificial
  Primer
ggtctagaggtcagtgcccccgcgtaccgtcgt

SEQ ID NO: 2 - DNA, 26 bp, Artificial
  Primer
ggcatatgcttgtgctcgggctcaac

SEQ ID NO: 3 - DNA, 36 bp, Artificial
  primer
cccgcccgcgcgagcggcgcgtggccgcccgagggc

SEQ ID NO: 4 - DNA, 36 bp, Artificial
  Primer
gcgtcctcgcgcagccacgccaccagcagctccagc

SEQ ID NO: 5 - DNA, 35 bp, Artificial
  Primer
ccaaccccgccgcgtccccggccgcgccgaacacg

SEQ ID NO: 6 - DNA, 34 bp, Artificial
  Primer
gtcgtcggctacgggccggtggggcagctgctgt

SEQ ID NO: 7 - DNA, 35 bp, Artificial
  Primer
gtcggtggactgccctgcgcctgatcgccctgcgc

SEQ ID NO: 8 - DNA, 35 bp, Artificial
  Primer
ggccggtggtgctgcccgaggacggggagctgcgg

SEQ ID NO: 9 - DNA, 39 bp, Artificial
  Primer
caccgctcgcgggggtggcgcggcgcacgacgtggctgc

SEQ ID NO: 10 - DNA, 38 bp, Artificial
  Primer
cctcctcggacagcgcgatcagcgccgcgcacagcgag

SEQ ID NO: 11 - DNA, 100588 bp, Actinosynnema pretiosum
gatctggggcgacgagccgcccgccgggccggggccggcgttgcaggcgctcgtctcccggctgcggcgggcgctcggcgcgccgggcgcggtcgcgctgggggtgggcgggtaccggctcgtggcggacgtggacgcggcgcggttcgaggagctggccgcgcggggcggggaggacgcgctgcgggaggccgccgcgctgtggggcgggcgggtcgggggcgagccgccggtggtcgcggccgtcgcgccgcgggtggcgacccggctggcgcggctgtcggtggaggtggtgctggacctggcggaggtcgagctggcgctcgggcgcaccggggcggccatcggtggggcgagcggggtgctggccgagcacccggcgcacgagcgggccgccggggtgctggtggacgcgctcgcgggcgcgggacggcaggccgaggcgctggcggcctacgagcgggtccgcgcggcgctggccgacgagctgggcgccgaccccggcacggccctgcgcgagcgccacctgcggctgctgcgcgccaccccgccaccgctcccccggccgaacgcgctgcccgcgccggtgacgggcttcctcggccgggacgccgacctcgcccgcgtcgccgacctgctggccgccgggcggctggtcaccgtcgtcgggcccggcggggtgggcaagacccggctggccgtggaggcgctgcgccgggaccgggacgcgctgctggtggacctcgcgccggtcgccgagccctcggaggtcgtcgccgccgtgctcgccgggatcgggctgcgcggcgaccgcgaccggccgggcggggacgcgacggcgctgctggccgccgagctggcggcgcgcaggtcggtgctgctgctggacaactgcgagcacctggtcgacgccgtggcccacctggtcgcgctcctgctcccccgctgccccgagctgcgcgtgctcgccaccagccgggaacccctggcggtcgacggggaggcgctggtcccgctggggccgctcgcgctgcccggaatcggggacgggcttgacgccgcggtcggcacggcctcggtgcggttgttcgcccaacgggcgtcggcggtgcgccccggtttcgccgtcgacgccacgacgctgccggacgtggtgcgcctggtgcgggcgctggacgggctgccgctggcgctggagctggccgccgcccggttgcgcgccctgccgctgcccgacctggtggccgggttgtcggcgcggttccgcctgctggcgggcgggaaccgggccgcgccgccccggcaccgcacgctgcgcgcggtgatcgcgtggagctgggacctgctggacgggcccgagcgggccgtggccgagcggatctccgtgctgcccggcggggtcaccccggagtcggccgccgccgtctgcgcgggcgccgtgcccgccgacgaggtgcccgaactgctggccgcgctggtcgaccggtcgctgctgagcctggtcgggggtcggcggcggatgctggagacggtgcgcgcgtacggggtcgagcgcctggccgccgccggggacttgagcgcggtccgcgacctggccgccgcgcacgtggcgggggtgctggcggggcaggacgcggtgctgcgcgggccggggcagcgcgcggcggtggcggcgatcggcgcggagcacgacaacgcggtggccgcgctgcaccaccggtgcgccaccggggacgcggacggggcgctcgcgctggcgctgtcgctggtctggtactggcaggtgttcggccgccagtccgagggcgcgcactggctcgggcgggcgctggcggtgcccggcgggccgtccccggagcgggactgcgcgcgggccgcccacctgctcggcctggccgacggcgggcacggggtgggtgatcgcggggaggtgggggcgctcgcggaccgggtgctggcgcaccgggggctccccggtcacctgcgggtgctcggggcggtcctgctgttcctgctggggcgcggcgagggggtgttccgggagctgggcgcgggcggcgggtggttgtccgggctggcgcacctgttcctggccgagctggcggagaacgcgggcgagctggaccgggcgcgcgggcacgcggaggtgtccctggaccggttccgggcggccggggacgggtggggcgtggcgggggtgctgccggtgcgggcgcgggcgcggcggtacgacgacctggacgggacgtgggcggaccttcgggaggcgcgggcgctggagggggagttcggggcgctgagccccggtgaccgggtgcgggcggacctgcggtgggtcgacctgcacgagcggcgcggtgacagcggggcggcgctggaggtgctggccgcggcccgtgctcggggggagcaggtcgcggtggtggacgcgcgggaggccgcgctgcgggtgcggctcggggacctggggcgggcgggtgagctgctggccggggtgggtggggcggtgggcgacctggcgcgggccgcgtatcgggtggcctcgggggacctggcgggtgcggagcgggcgttgcggcgggcgcgggtggtggcggctgcgagcggggagctgcccgcgctggccccggtggcggtgggggcggcggcgctggagcaggcgcgggggcggtgggcggggtcgggggtgctgctcgggacggccgcgcgggtgcggggcgcgcacgaccgcaccgaccccctggtgcgcgagctggtcgaccgggggcgggcggcggtgggcgggagcgcgttcgcggcggcgtacgcgcgggggtgggaggcggagcgggacgtggcggcggcgttcgtgctctgagcgccgggatcgggcgggcggggtcaggcgggcggggtcatgtgggcggggtcaggcgggccaggtcacacgtccagggaccccgcccagtccgcgatcgtccggacttcggcctgcgtcgggaagaccttctcggtgagcacgcggtgcacctcggggtcgccgtccaggcagccgtcggccaggacggtgagctggaagtccaggtcggcggcctggcggagggtggacaggaccacgccgctggtcgcgatgccggtgagcaccaggtggtcgacgccctgggcgcgcaggacgaggtccaggtcgctgcccgcgaacgcgctgacgcggcgcttggtcaccaccacctcgtcgtcgagcggcgcggtctcggggtggaagtcggtggcgccggagcccctgggggccgcggccaggcggccgaacatcttgttgcgcgggtggatctccgcgtagtcggggcggaagccgacgccgacgtggatcaccggcacggacgcggcgcgggccgcctcgagcgcggtggcgagcctggggaggtaggccgggtcggggtagcgggcgaccacggcgggctggacgtccatcaccagcagggcgggggtggggatctcgggcctcgtttcggtggtggcggcgcgggggccgccggtgggggtcaggggtgcgggggtgccggggtgagcaggctggtgacggtgagcaggcggtcggcgagttcctcggggcgcagcgggtcctcggcgcgcacgacccagtcgtggacgatcgcgccggtgccgtgggagatcagcacggccaggtcgcgggcggcccgctcgtccacctcgcccaggccggcgcgcagggcctcggtggtgatgagggtgcggaagagctcggccagggcctccgccagccgccacgcgcacgggccggtgagcacggcgcggtagaaggggcggtggtcggcgaagtggcgggccacggccaggaggcgggcgtggcgcggggcccgcgggtcggccaggtgcggcaggagctcgcgccgcaccaggtccgccgcagcggcgacgaggagcgtgtcgcggtcgccgaagtgctggtagagcagctgcctgctgacgtcggcggcctcggccaggtcggtcaccgggaccgccgccccgcgctcggcgaccaggtcgacggcggcggccatgagggcggccctggagcgggcgacccggcggtcggggcgggtggtcacgggggtgaaactagacagttgtcaataaatgagcaagtgtcgtcgaacgcgcgcgcgggaatctccggtgcgcggggcccgtccctggcagcatgatcacgcgatgaccgaggtgaggacgcgcccgtacgccgggcccgcggacctgcgcgcgatgcaggggttggcgcggcggatctggacgccgtcgagccggtggcacgtcggcgacctggcctggcagcgcaaccagcacaccgggcgcgaggccgagtggccgaccgcgctgtgggaggcgggcggcgaggtggtggcgtgggggtgggccgagctgccgggtgagctggcgctgctggtcgaccccgcccggccggagcttgcgggggcggtgctcgactggttcgcgggcgtggccaccgcgccccggcggtcggtcaccgtgctggacgccgaaccgcacctggtcgccgcgctggaggctcgcgggtacgagcggctgggcgggccgcacttccggcactcggtgcgcgcgctggacgacctgccgacgcccgaactgcccgccgggtaccgggtccgcgccgtgcggggcgaggaggacgtggcggcgcgggtcgcggcgcaccgggcggcctggtggccgtcgcgggtcaccgaggagagctaccgggcggtgatgggggcgtggccgtaccggccggggctggactgggtggtggaggggccggacgggcggttcgcggccacctgcctgatctggttcgacgagcgcaacggcgtgggcgagctggaaccggtcggggtcgaccccggtctgcggcggcgcgggctggggcgggcggtgtgcctggcggcgctgggcgcgctgcgcgaggcgggcgggcgggcggcggtggtgtacccgctgcacgggcaccccgaccaccccgcgcccgcgccgctgtaccgggggctggggttccgcgagcacgcccgcacgatcaccttcaccgcgctggaggcgcgcgggtagcagcggccgggcggggcgagcggacccggtcgacgagcggctccgctgtcggagcggtcgtcccagcgcgtggacaccagtgccacgaccagaccgcgccccgcttcgcgtggtcggctcgggggtcgaccgcggtgaggctctcgccggggtgggtgaaccacgtcctggcgatggcctgcaccgcgagcaccgggtgccgcccgtggcgctggacgtcaccgacgcagccgccgtcgaccgggccgggccggccgcgttcggccgttgcgccgcgccggccgagtccgacgccaggtggcggccggtccccgggtccgcctggaactgaccccgccggcctccccgcccgcccgtccggccgggcgccgaacccgcctcaggcgtgctcgaccgcgcgcaccgatccccccaccaccaccggcatcgggacgtggtgcacggtcgtcgggctgcggtcgcggcgggggcgggacaggaggagttccacggccatcgcgcccaggcggtggtgcggcagggcgacggtggtcaggcgcgggcgcatccaggcggccacggggtggtcgtcgaagccgaccacggagacgtcgtccggcacggacaggcccgcctccgcgagcgcctggcacgcgccgaacgccaggcggtcgttgaagcacagcagcgcgcgagggcggtggtgggacaggaggtccagggtggcgcggtagccgttctccggcatccactccacgcacgggcgcacgctctccacctccacccccgccgccgcgaaggtctccagcgcgccggagaggcgggccacggcggcgatgtggcgcgggtcgatcgcctcggccgtgggcccggtgccgatcaggtgcacgccctcgcggtgcccggcgtcgagcagcacgcgcgccgccgaacggccgccgccgcggtcgtcggggagcacggcgtgcgcggggaagtcgttggcgggcagcacgttcagcagcacggacggcccgtcgcgcagcccgtccgggacctccagcagccgggggaacctggccgcgaagaccacgccctccacctggcgggcgcgcagcgaggccaccagcgccgcctccacctcgcggtcgccgccgctctcaccggcgaacagggtgaacccgtgccggtgggcggcgccgaccgcgccctcgatcagctcaccggacagcttggccgaggccacggcgtccgagacgaaaccgagggtcttggtgcgggaggcggacagcagcgtgtcgcggcggtagccgagctgctcggccgtcgcccgcaccttgcgctccaccgccgccgagatgcgcagctcccgagcgcggccggagagcaccagggaggcggtgggcaccgacacggcgcaggcggacgcgacgtcggccagcgtgacgcgcgtccgcccgctctcgcggacacctgctcgcgggggtgtgcccgtcacccgtgcctcccgtcaccggtcgcgcgacagccccgcgcgaggtcctaccccatcgtgcaggccgcgccgttcaaggagaaccccgaaggtggggccgcgtccccgccgtgggtgacctggtagccgatgctgactttgccaccgggtgggatcgccgcgttgtagcccgcgtcgcgggcggtcacccggcccgagctgggcgcgtacgaggcgttccagccggaggtgatcacctggcccgcgggcagcgcgaactccagcgaccagccctgcacctgcgtggtcccggtgttggtgatggcgagctccgccgtcaggccgttgccccaggcgttgacggtggccgacacccggcaggcccccggctgcggttcgccgggcgtggtggtggtggtggtggtggtggtcgtggtcgtggtggtgctggtcgggtcggggccggttccggcgaactgggtgaagaaccgccaggtctcctcgggcgcccacgtcctggtgccgctgtcgccgggcgcgttgtcctgcggtgcggcgatgtggccctcgtcgaacgcgacccagcgcaccgggtagccgtcgcggcagccggtgtaggtggtgccccggtgggtcaggctgccctgggacggttccggcgggttctgcgcggcgcagccgttgttgcgcacgaaccggtcgcgcatcgagcgcccgccggagatgttcaggacgctgtcgcgcaggccgtggatgccgaggtaggcgatgggctgcgtgccgccggcgcagccgctgagcacgccgcccgcgatgaccgcgaccgcgcggaacaccgtcggccgcgagcaggccaccgagtaggacatcgcgccgccgtagctgaagccggtggcgaaccgctgggtggtgtccacgcacagcccggcgtcgagctggcggacgatgtcgtcgacgagggtgatgtcctcgccgccgttgttggcccagccgttgttgaagccctgcggcgccacgaagatcgtgctgctgcccgccaggcgcttgaggccgtagtaggaccagacgtcccgctgcacggtctggccggtggcgacgtcgttcgcggtgccgctgagccagtggaagccgaagacgacgcggtgggggcggttccggtcgtagccgtccgggatcgacaggatgtaggtgcgggacttgccgctgctggtgatcgtgcgcgtgccgctggtgagcgcgggcgccttgccgcagccctccgtcgtggcggacgcgccgggggcgccggacgcgccgggcgctccggtcgcgctggtggtgatcagccccgcggcgagggtgagcagcgcgatgcccgctgccgcgaggaccctgttgcgcgccaagggattcgcccttcctgtggtggttccggtgggtgtggtcacggggtggtgaggtcgaagcggcgggcggtgacggagccgccgagcgcggcggtggcgtggttgaagacggcgaagcggtagcccatgaagaaccgccagtcgttcttgagcgtgaacgccgggccgaaggcggtgaagttgacgccgtcggtgctgtaggagaaccgggcctgcctgccgttgccggggcggatgtcggcgttggcgcgcaaccagatccgggagccgcccaggtcggcgctcgcgacctcgtagccggttccggtggtgcgccaggagccgtccatggtcaggccggtgacggagacgatccggttgcggccgttgtcgcgcttgacgccgatccacgccgaggagtcgcgcagcacggccagcccggtgcggtcgccgtcgcgcatccccgacaggtccagttccacggtgccggtggaggtggggccctggatgcggtgggtgagggtgttgcgggcggagtacaggtcgttggtgacggtcgcggtggacaggcgaaggccgttgttcacgctgtacttggcggtgtccgggttgtggttccactcccactgcgggccgagcgcggcgccggagaaggtgtcggcgccgatcatgggtttgacctggcgcgggggcgcgggcaggttcggcttcgggtaggtcgcgccccagccgccgttgacggtggtgacgcgcggccagccgtccgaggtccaggtgatcggggcgagcaccggcacgcgcccgccggggtaggcgtcgacgaacgccaggtagtgccagtcgccgttctgggtctgcaccaggccgccctggtgcggcactcccccgccctggatcggcgagggcaggtcgagcagcacctgctggatcgagtacgggccgaacgggctggacgacttgagcacgtactggccgttcgcgggcctggtgagccagatgtagtagttgccgccgcgcttgtagaagcgcgccccttcgagggtgccgatgttcgagggggtctggaacacctgctgggagcggacctccgacttcccgtcggcggagagctgggcgacgctgatgctggtgttgccgtaggcgacgtacagggtgtcgtcgtcgtccacgagcatcccggcgtcgtagtagcacttgttgatggtggtgtgcttggaccactggccgtcgacggcggtcgcggtgtacaggtgcgtctgggcgaagtcgacgcagccgccccagtagaaggtgcggttgctcttgcggtgcgccaggaacgacgcccagatgccgttgacgtacgcgcgggagccgttgcccatgtcgtacttggccccgaagtccaggcgtggcacggagtgcccggcgaactcccagttgaccaggtcgtaggagcgcagcacgggcgcgccgggcgagtagtgcatggtggaggccgagtagtagtaggtgtcgtccacgcgcaggacgtcgatgtcggcgaagtcctgccacagcacggggttggtgtaggtcccggccgcgcgggccgggtgggtggtggtggcggtcaggctcgccgccacgaggccgagcacggccagggcgatgggcgcccatcggcgttgacggggcatcggtgtgcctctcctggtgtccgggagttggctctgggcgcggcggcggtggacttgtcgggcgcggcggtggtcgtgggggtcagcagggggagttggtctgggtcagcaggcccagccgccagggcaggcggttgtagtcgccggacgcgttggggtccaggccctggtacaggtagctcagcttgcaggggttgatctccatggtctggtcggtcccgctgcgcaccagctcgccgtggctgatgtcgcgcgtccactggccaccggggaacgtggtgttgttggccctggcgaacgggttcgactcgctgtcggccagcgcggtccacggtccggcgatcgccggggcggtccaggagcggaaccagcggcggccgtccgagccgatcgcctcgtggagcatcagccactggttcttgccggcgaccttgtagatgttggacgcctcgaacaaccggttgcggttgctgtcctgcatggcgatcacggtgttggtgaagccgttggggaactgggcgaggctggtctccgagcggtacaggtggccgttgtcgtccgaggagaacaggtggcacttggccgtgtcgcagacggtccagaagtcgacccagtagccgttgccgatgttgtcccggatgatctgcggcatcccgttggcgtagaagttcctcggcgcggaccaggacgcggggttctcgatgtcggcggtcgtcgagtacgaggcgttggacccggtctggtacaccaggtaccacaggcgttgcggggcgaagtagaacacctgcggcgcggcccggtagcccgtgccgatcccggagcggtccaggtagtggtgcggggcggacgcggcctgggaccagtcggtgaagctggtgtgcacgaggttgtagccgttggtgtagaccgaggcgaacacgtggtagcggccgttgtggcgcaccacgctggggtccttgacggagaccgtggcgtgcgaggagtcgggcttgggtccgatcagcgcgccgctggaggaccaccggaagctgctcggcagcgagccgcccggttgctgcggggtcgtggtggtgggcggcgtggtcgggggcgtggtggtcgtgggcccgactcctcccgtgcacgtggtcccgttgaggctgaacgaggtcgggtcggggttggacccggtggaggtcgcggtgaacccgaactcgacgcggcccccggtggggatggcggcgttgtaggaggcgttgcgggcgctcacctggccgccggactgggacacctcggcgttccaggcctgcgcgacctgctggcccgagccgtaggtccaggtgagcgtccagccgtcgacggcgtcaccgaggttggtgatggcgacgctcgcggtgaaaccgccctgccactgggaggtcgccgcgtaggcgatcgagcaccccggcgccgcggcggcctggggtgggagggcggcgagcgcggccaccatggcgagcgaggtggtggccatggcgccgatccgggcccggcgcggggtgaacagccttgcgaggagcatggtcgcccttcgtcgtcgtcgacgggtggtcggcgcggccgaccgggagcggcgggagcgggctggtactcccccacttcctgcaatctagccaggtggcacagggtggtcaaagctaaaaagggcggacgcggtttagcttccagcgcaaaggtttcgcgcgttctttcggccggggggcaggtggatcggggcgggctcggggcgaggacggggctgggaatggggcgggggatggggcgggctcggggcgggggtcgcccggagcccgccacgggtcagaggcgcacgcggacgacggtgaacgggaggttcggctcggcgatctggtactggaagttgaccaccagcaggtcgtcgccgtcgaaggtcgcggtggacgggacgtccatgcccctgccgttgacccgccgcagcaccgtggcgcgggagtggtcctcgctcagccgcaggacgctgatctcgccctccgggtggaacaggctggtgacgctgtagaggtcgttgccgcgcaggagcagcccgtccgagccgatgtcgcccacgccgcccaggtcgatcggggtgacggcgccggtgcgggtgctgatgcggtggaacgcctgggagttggtgtcggcgagcagcacgtggcggccgtccggggtgaccacgaggccgttgccgttgatgccctcctcgtagcgcaccggggagtcggcgaggtccacgaacgtcctcagtggctggtcgacctcggggctcgccagctgggcggcggtgatccggtagaggacggggcggaacgagtcgctgacgtaggcgtcgccgttcggggcgatggcgacgtcgttgaccaggccgtcgcgggcgccggagtcgaacacgtgcaggagcgcgccggtgcgggtgctgtggacgaagaccttgccggtggcgccgcccgcgatgaccagcctgcctcgggtgatcttcatgccgacggcggtggtgcggccgtgctgaccggcgggcaggaacggctccagggcggggcggtcgacgtggccgcgccagatcgtgccgtcggtcgtgccgccgacgtagaagtgcggcgtgcccggctcgcggacgatgccctccgggtaggcgcggtcgccggggaccacgtagcgggtgacggggtggtgcgcggcggcggcgggtggcacggcggcggcgggtggcgcggcggcggcgggtggcgcggcggcggggagggctggggcgagcgcggtgaggagcagggtcgcggtgaggagggctcgggtggtggtcacggaagggctccgggggtcgaaggggtgtctggcgccagacaagcgcttcgtggcgcggggtggcagtgggcgcttgtcgggggtagttcttcaccccccttccgggcggggcggccgactagggtgagcggtgtgggcgatcttgggcggcacccggtggcgaaccgctccgacgtgcgggacttcctggtcagcaggcgcgcgagggtgagtccggggcgggccgggctgcccgtgctcgggcggcggcgggtgccggggttgcggcgggaggaggtcgcgctgctcgccggggtcagcgtggactggtacacccgcttggagaaggggcacatcggcggtgtctcgcgggaggtgctcgacgccgtggccggggtgctgcggctcgacgccgaggagcgggtctacctgttcgacctggcgcgcgcggcccggcgtccccgcgccgccgaggtggcggcggaggccgcgctgcccgcgacggcgcagtggctgctggacagcatgacgctgtcgtcggcgatggtgaccgggcggcggcaggacgtgctggcggtcaacccgctggcccgcgcgctctacgcgccgctgttcgccagcgccaccacgcgggacggcggccgggcgaacctcgcccgctaccacttcctcgacgcgggcgcccgcgagttctacggggactgggcgggcaccgccgacgtgctcgtcgccgcgctgcgcgccgaggccgggcgcgacccgcgcgacggggccacccgcgagctggtgggcgagctgacggccgcgagcaccgagttccgggcgcggtggagcgcgcacgacgtgctgctgcacccgcgcggcgccaagaccttccggcaccccgaggcgggtgagctgagcctgagctaccactcggtggacctgccgatctccgccaccgagacccggcacgtgtgcgcgtgcaccgccgaacccggctcgaccgacgaggcgaggctgcgcgcgctcgtcgggtgagccgggggtggccggccaccgccgtcgcgctcgcggcggcggggcggggccgccggtcagagcgtgagcgccatcccgatcgagccgggcgcggtctcggtgaagccgtgcttgaggtacagcgggcggccgggcgcgtcggccaggagggtcacgaacgcgccgggcggggcggcctcgcggatgcgccggagcagcgcgtccatgatcgcgccgccgacgccccttccctggtggtcgggcagcacggccatgtcgacgacgtggaagtaccagccgccgtcgccgaggacccggcccatgccgacggtcccgccgtccgcgtgcgtgacgtggaaggaggcccaggcgccgggcagggcggcggcggcctgctcggcggtcttgggcgacaggccggactcggcgcgcaggcggaggtagtcggcgacggacggcggggtcgggtggagctcgtagtcccggtgcacgcggtcaggctcccacgggcgcgggcgggcccccgcccgacctgacgatttccccgtcggcggggatgcgggcgggcgctcgcggattttcgacatcccccggcccggcgagacgcggcggcgccgctgaaaagagcgccgtcgcggcccttcgcgccgcccccgacatcccccgcgcggcgaccggtcaatgcggtccacgcctgggggtttccctcccacgtcgaacaccgccaccacgcgcccacgcgccgcgtcgaccaccccgacgccgaggaacacctgttcacgggcacgggaagccgcagcggagggggaaccgggaatggccgcaggcgatcgcggcacgacgtccgcacatcaccgcgagcagaatcgcgaggcgttcaccggggcggcggaggaagattccagcgcccctctcgaagaacctgcgggaagccctggaagaaaacccggacccgaaacgcgacaaaattgcggacacccacccgtgaaacaccgggcgcccccaccaggtcacccgctgacatcacgctcagtcagtatcggcacgctcccccgccgaggcggagcgcgacaccccgcccaaccgggcaccgagcgggcacctccactcggcccagcaccgccccaagatcgcacgtagcacgggttgaaaccgctcaagcgcatctcaacccgttcggagcagagtggcgcccgtcacgtcccgaccggtcacggttggcaacgggtccagtccacgcgaggtggcatcaagcgcacttgccccgatcacacccgcccgtgcaaccgaatgcagcagggatatctttcccgagaactcggccgttaaccgggagtggagccaggcccacccctaagacgcttgcccacatgcccaacaatggtgaagatggaacggcggaccgcaccgcgaacgcgaaccgaactccgcgagagggcacggtgaacgatcctggaacagctactgccgcgtagctcaagggtggaacgcccggctcgcgcgcggcggagggaataacggcttttacgccctcgacaacagcttgtcaacgaaacccgtgcacccgagcggtccccgcgcgcaccgctcgcgggggtggcgcggcgcacgacgtggctgcccggcgtcgacgacgacgcgagttccccgaccgcccgcgaaggcggtcgcggatcgccacgacggggcacccggaccacgcctcccccggaacagcgcgcccacgcgcggttccggcgcgcgccgggaccgccgcacccgccggagcgccgccaccggccggggccggtccccgcggaccgggtcgccttccggaccaccactccacggaccacggaaaggaccactcccccagtggagcttctgcgcgcacccgagatccagtcggccgtcgagcacctcgcggtggacctgccggaccgggcgggacgggcgttcctggtggacggaccgcccgcctgcggcaagacgacggccctgcggcggctcgtcgaccggatcgcccacgaggaccacctcgtgctcaccgccacctgcaccccgccggagacggagctgccgttcggggtgctcaagcagctcctcgcctcccccggcatggccagggtcgacccgcgcctggtcgccgacctcggcgagctgctcgccccggccccgccgcccgccgacgactcggcgctcctgcagctgtaccactcgctgtgcgcggcgctgatcgcgctgtccgaggaggtgccgctggtcatcgcggtggacgacgtccgccacggcgacaccgcctcgctgcacgtgctgctgcagctggtgcaccggctggacacggcgcgggtgcggctgctgctcaccgacgacctgctgctgccggtgagcttcccgccgctgcgctacgagctgctgcgcctgcgcgggctcggcctggtccgggtcgcgccgctgcctgcggccagggtgcgggaggaggcggtgcgggcggtcggcgcggacgtcgcgaagcgggtcgacttcgccgcgctgaccggcggcaacccgctgctgctgcacgcgctggcggtggacgtgctggaggcgggcgagccgcgcgagatcggctacggcaactcgttcctgtcctgcctgcaccgcaacgaacccctgttcctggacaccgtgcgggcgctggccgtgctgggcggcggctcggcgtcggacctgggcaggctgtccgggcacgagccggagcaggtcgcccaggtgctgaacgccctgcgggagtcggggctgctggccgaggacgggttccggcacgacgcggcgcgccgcgcggtcgtcgcggacaccccggtcgccgagcacgaggtgctgcaccgccgcgccgcgcggctgctgcgcgaccagggcggcgcggtcaccgacatcgccgaccacctgctgcgggcgggccgcatcaccgacccgtgggcggcggacctgctggtggacgcggcggagctggtggtgcagcgcggcgagccgacggcggcggtggcgctgctccagcgcgcgctcgactgcagcccggaccgggagcgcaggacggccgtgcaggcgcggctggccacggccgagtggctggtgaacccgtcgacctcggcaaggcaccacaccgcgctgctggcggcgttccacgcgggcaggttgtcggtgcgcgacagcgcgacgctgatgaagcacctgcgctgggccgggaacaccgccgactcggacgcggtgctcgcccggctgcggaccgacccgcgcgccgccgaggacgtgccggtgctggagcactggctgaccagcacctaccccggcgcggcccggcccaggaccgtgctggggcgggacgtggactcggcgcgcagcagggcggacctggtgccgagggcgaacgcggtgctgctggacgtgctggtggccggggacagcgacgacgtggccgaccgggcggaggcggtgctgcgggagctgcggctggcgcgggagtccgggtgctacggcggtgcggccgtgctggcgctgtccgcgctgctctactcggaccgcgcggacgtggccgcctcgtggtgcgagcagctgctgtcggcgcgggccgtgccgctgctgccgatgccgcgcgcgcaggtgctggcgctggcggccgagtcggcgctgcgccggggcgaccacccgagcgcggacgagctggcgcgggaggcgctgaccgtggtgtccccgaccgcgtggggggtgtcggtggggctgccgctgagcaccagggtgctggcgctgaccaggatgggccgctacgacgaggcggcggccgtggtggcgcagccggtgccgaacgggatgttcgggcaccgcaacagcgtggactacctgtacgcgcgcgggcacttcttcctggcgcgggaacggccgcgcgcggcgctgggcgacttcctgctgtgcggggagcagctgacccggtggggcctgggcagcgggtgcgcgccggtgccgtggcggaccgcggcggcggaggcgtggctggcgcagggcaaccgggaccaggcgcgggtgctgatccacgagcagctcggcaggccgggcacggacagccgccgggcgcgcgggcaggcgctgcggctgctcgcggcgaccagctcggtgaagcggcacccgcagctgctgcgggaggcggtggcggtgttcgaggccgtcgacgacaagtacgagctggcgcggaccctgcgcgacctggggagggcgcagcgggcgctgggcgagaacaagctggcgcgccgggtgatccggcgggggtggcacgtcgcccggatgtgcgaggcggcgccgctgtgcgaggagctgatgcccaccgccgacgggctggtgcccgcgcagcccgcgtcggcggcccgcaggtcggacctggaccggttgaccagctcggagcaccgggtggccgcgctcgcggcgtcggggctgacgaaccgggagatcgcggtgaagctgtacgtcacgcacagcacggtggagcagcacctgacgcgggtgttccgcaagctcgggatcaagcagcgggagcagctgccgccggagctgagcgtcgaccggtcgaagtgacgcggacggggcggtcccgtggatctggggccgccccgtccggtcccggtccgtccggtcccgccctgtccggtcccgccctgtccggtcccgccccgtccggtcccggtccgcctcaggctcggggcatcgcggccagggtggtggcgacgacgtgctcgtcgacgtcgcgcaccagctcggcgccgcgcgggccgtcgagcacgaacgccaggccgccggtggacttcttgtcgcggcgcatgaaccgcagcagctcgtcgtccggcacgcccggcggcagcgcgacgggcagcccgtagcccgcgaccacggagtggtgctcggccacccggtccgggccgatccggccgagcgcgcccgcgaggcggccggcgaagaccgtgccgatcgcgacgccctcgccgtgccgcaccgcgaaaccggtggcgagctccagggcgtggccgagggtgtggccgtagttgagggtgtgccgcaggccggagtcgcgctcgtcggcggccacgacgcgggccttgagggcgacgctcgccgccacctggtccagcagcggcagccggtccaggcccggcgcgccgatgaagtggcagcgggcgatctcgccgaggccgttgcgcagctcgcgctcgggcagggtggcgagcaggtcgaggtcgcacagcacggcggcgggctgccagtaggcgccgacgaggttcttgccctcggggaggttgaccgccgtcttgccgccgacgctcgcgtcgacctgggccagcagcgaggtcggcacgtgcaccaccggggtgccccggtggtagagcgaggcggcgaggccgaccgcgtcggtggtggtgccgccgccgcaggagacgacgacgtcggcgcgggtcaggccgaactcggcgaaccggctgcacaggtgggcgacggtggcgagggtcttgtcgtgctcgccgtcgcgggccgggaggacgagggaggggacgccggggtcgggcgtctggtccgccgggcgggcggtgaccacgacggcgcggcgcgcgccgagggcccgcacgacgtccgggagggcggcgcgcacgccgtgtccgatgtggacggtgtaggcgcgctcgcccagctcgacccggacctcgcgggtggtggcggcggtgggggcggggtgggtggagctgggcactgcttcctcctcgggtgggcgggacggggggcgatcgggggacgcggaggggtgacgggaaagcaatcgggcaggaatgggaacgggtccgggggcgaacgggcaggaattcgaatgggggcaagcgaccgggagcgatcccagtggtggggcggaagtgcgggcggcggaaaggcggtcgtgctgcctcagccgccgcccgcggcgcccgtcacgagcgtggtgcgcagggtgagcgccgcccgcgccgccacggccgcgccgacgccgaggcggttgtgggtcagggtcgcctgctcgaacagcaccggcagggccgggcggcggcggtccgcggcggcgcgcagctgctccaggtagcgccgccacgcgcccgccgagccgaccacgcgcccgccgggcggcgccccctgcgcggcgacggcggcctcggtgcgctcgaccgggcggagcggcctgctccagctctgccagcagtggaacaggaacgcgggcctggccggttccggggccagcgcgaccagttcggccaggtggtgcagcaccgtggcctgcgcgcccgactcgccagggtcctcgccccacaccgggctcaccaccgacgcgacgtccagggccagttcgctggacgcggtcgcgaccagcggctcgaccggcccgcccggcaggcccccacccggcaggccctcgcccgccgggtcgacgcgcaggtcgcgggccgccgcctcggcgcacagcacggcgagcaccgggccccgcgcggcctcggtcgtgcccagccacagcgccaccacccccgccccgcgcaggaacgaccagtgcgcgccccggtcccgcgcgcgcagctcccgcacgacggggggcagcacctcggccaccgagcgggctccaccgcctgccagcgaccagcccgaccaggacggggcgcccgccgccgggggtccgccgaggggcgctctcgcggaaccggtccgcgtcatgtccaccaccacttccgccttggcgagaacgggtcctgcgggatcaccgcgctgttccgacgccgccgacaatagcgacgcgcaatacgccgaattcaccgccaaatcaggtcaggggggttgagggggatgccttagggggcgagtgcccgcaaagcggaagaagaatcggaagcacatgcaggagcgacttccaagctcaggccgcaggaccgggtccgcgtcgtcgcggacaccccggtcctgcgcgtgcgcgcaccgaaggacgtggtgacatgcttcggaccgacctgatccgcccggttcccgaactgctcggggccaacgcggatcgcttcggcgacaggaccgcctactcggacggtcgccgttcggtcgggcacgccgggctggaacggcgcacgcgccgcctcgccggtcacctcgggcagttgcggctgcaccccggcgaccgcgcgatgatctgcctgggaaatcgcgtcgaaatgatcgagagctatttcggcgtgctccgcgcggacgccgtggcggtcccggtgaacccgcgttccaccgacgcggagctgacccacctgctcgccgacagcggggcccggctggtgatcaccgacgcggcgcgcgccgagcggttcgaccggttgcgcgccgagcggttcggcgacctgaccgtgatcgccacccaggacggcccgctgcccgacggcgtcatcgcgttcgagccgctggccgccgaggagccggagctgcccgcgcgcgacgggctcgggctcgacgacgtggcctggatgctctacacctccggcacgaccgggcgccccaagggcgtgctgtccacgcagcgcagctgcctgtggtcggtggccgcctgctacgtgccggtgccggacctgcgcgccgaggaccgcgtgctgtggccgctgccgctgttccacagcctgtcgcacatcacctgcctgctggccgccacggccgtgggcgcgaccacgcgcatcgtggacggcacgtccgcgcaggacgtgctcgcggcgctggagcaggagcggtcgacgttcctggcgggcgtgccgacgctgtaccggtacctggtcgacgccgcccgcgagcgcgggttcaccgccccggacctgcgggtgggcctggtcggcggggcggtgacgacggcggagctgctgcgcgcgttcgaggacacgttcggcgtgccgctgatcgacgcctacggcagcaccgagacgtgcggggcgatcgcggtgaactggccgaccggggcgcgcgtggcgggctcgtgcgggctgccggtgccggggctgacggtgcggctggtggacccggagacgctgctggacgtgcccgccgggcgggagggcgagttctgggtgtcggggccgagcgtgatgctgggctaccacaaccagcccgaggcgacggccgaggtgctgcgggacggctggtaccgcacgggcgacctggggcggcgcgacgaggccgggttctgcacggtcaccgggcggatcaaggagatgatcatccggggtggggagaacgtgcaccccggcgaggtcgaggccgtggtgcgggcggtgccgggggtggcggacgtcgccgtcgtgggcaagccgcacgacgtgctgggcgaggtgccggtggtgttcgtggtgccgggcgcgggcgggttcgacccggcggcggtgctggcggcgtgccgggaggagctgtcgtacttcaaggtgcccgaggaggtctacgagatcgagcgggtgccgcgcacggcgtcgggcaagaccacccggcacgtgctgctggacctgcccgcccggttgcgggcggcgtcgagcgggcagttccagtcgctgctgcggctggactgggtgccgaggacggcgctgccgggtgaggaggtcccggcgagctgggtgctggtggacggcgacccgctggggctcgcggacgggttgcgggccacgggcgcgcgggtgcgggtgggcgagccgggcgcggatgcgctgggcgacggcggatcggacgccgacgagccgggcgcgagcagcgcgggcgaaccgggctcgggtggctcgggtgagccgggctcgggtggctcgggcgaaccgggctcgggtgaaccgggcgcgagcagcgcgggtgagccgggtgcgggtgagccgggcgcggccgaacccccgcaggtcgtgctggtcgccgcggtccccggtgagcgtggtgaggtcgcgcgggacgtggaggcgctcgcggacgggctcgcgcggcggctcgtcgggtggctggccgacgagcggttcgcgggggcgcggttcgtcgtggccacctcgggcgcggtgtcgacctcccccggcgaggacctgcgggagctgcgggcggccccgctgtggggtgtggtgcggtcggtgcaggccgcgttccccggtcgggtggtggcggccgacctggacgcgtccggcgacgggcgggcggcggcgctggctcgcgtcgtcgcgggcgggcacgaccaggtggccgtgcgcggcgacgtgccgctggcgccccggctggccagggtgtccgtgccgtccgacccggcccccgccccggcgctggacccggacgggctggtcgtggtcaccggtggcgactcggcgcgcggcgcggccctcgcgcggcacctggtggccgcgcacggcgcgcggcgcctgctgctggtctcccccgacgggctgcccgaccaggccgccgccgacctggaggccgggttcgcggcggcgggcgcgcgggcggagtcggtggtgtgcgacccggccgacccggtcgcgctgcgcgccctgctcgacgcgcaggaccgcccggtcacggccgtggtgcacgtgcagggcgggcgggcgctgctggactcggcgcgcgccctcgtcgccctgcacgagctgacccgccaggcgcgaccggcgctgttcgtcgtggtcacctcggtggccgggctgctgggctcggcgggcgacccggcgcgcgcggcggccgaccagttcgccgaggcgctggtgcgcaggcgcgccgaccggggcctgccggggctggccgtggcctggggtccgctgccgggcgagcccgcgcaggcgggcgcgggcgcgctgccgatggccgaggcgctgaccctggtcgacgccgcgctcgccgccgaccagggcccgctggtggtgctcgggctcgacgcggtcgggtcgcggcgcgcggtgggcgcggtgccgccggtgctgcacgacctggtcgacggcggtcgcgccgcgcgggtcgcgccgggcgcggtggccgagttcacgcgcaggctcgcggaggcgggtgggcagcgggcccgacgcgtcgcgctggacctggtgcgcgagcacgtcgcggcggcgctcggcctgcccgaggacaccccggtgcgcgccgaccaggcgttccgcgacttcggcgtcacctcgctgaccgccgtggcgctgcgcgaccggatcaacgccgcgaccggcgcgtccctgcccgcgacggcggtgttcgaccacccgaccccggccgcgctcgccgaccacctggtgcgcgaggtcaccggcgaccggccgcacgtcgagcgggcgcgggacgagcgggcgcgcgggacctcgcgcgcggacgagccggtggcgatcgtcgccatggggtgcaggctgcccggcggcgtggcctcgccggaggacctgtggcggctggtggacgagggcgtcgacgcgatcggcccgttcccgaccgaccggggctgggacctggccaccctgctcgacggctcggactcgccggggaggtcctccgtggaccgcggtggtttcctgccgggcgcgggcgacttcgacgccgggttcttcggcatctccccgcgcgaggccctggccatggacccgcagcagcggttgctgctggaggtggtgtgggagaccgtggaacgcgccgggatcgacccgcgctcgctgcacggcgaagacgtcggcgtgttcagcggcctgatgtaccacgactacgggaccgaacccggttccgcgccggagggcctggaggggttcgtcagcaccggcagcgcgggcagcgtggtctccggccgcgtcgcctacgcgctcggcctgaccggcccggcgctgaccgtggacacggcgtgctcgtcgtcgctggtggcgatccacctggcggcgcaggcgctgcgctcgggcgagtgctcgatggcgctcgcgggcggggtcgcggtgatggggcagccgacgtcgttcgtggagttctcccggcagcgcgggctcgccgccgacgggcgctgcaagtcgttctccgacgacgccgacggcacgaactgggccgagggcgtgggcgtgctgctgctggagcggctctcggacgcgcgccgcgacgggcacccggtgctggcggtgctgcgcggcagcgcggtgaaccaggacggggccagcaacgggctgaccgcgcccagcggcccggcgcagcagcgggtcatcaggcaggcgctggcgaacgccgggctgcgaccgtccgaagtggacgccgtggaggcgcacggcaccggcaccaccctgggcgacccgatcgaggcgcaggcgctgctcgccacctacgggcaggaccgcgagcagccgctgtggctgggctcgctcaagtccaacctcgggcacgcgcaggcggcggcgggcgtcgcgggcgtgatcaagatggtgatggcgctgcggcacggcgtcctgccccgcaccctgcacgtcggcacgccctcgtccaaggtcgactggtcggcgggcgcggtcgagctgctgaccgaggccaggccgtggcgcgcgaacgggcggccacgccgggcgggcgtgtcctcgttcggggtcagcggcaccaacgcgcacgtcgtggtggaggagcaccgggaaccggccgccgcgccggtcgacccggtctcccccggcctggcggtcagcggcggcgtcgcgccgctggtgctgtccgggcgcacccgctccgcgctcgccgcgcaggccgcggccctgctggggcacctggccgacgggaccgacccggcggcgctgggccgcgcgctcgccaccacccgcaccgcgttcgagcaccgggccgcggtcctcgcgccggacgtcgacgccgcgcgcgccggggtgcgcgcgctcgccgaggaccggcccgcgccgaacctggtcaccgggcaggccgacgtggacggcccggtcgtgttcgtcttccccggccagggcgcgcagtggaccggcatgggccgggagctgctggagacctcgccggtgttcgccgcgcggctgcgcgagtgctcggaggcgctggagcggtggaccggctggtccctgctcgacctgctcgccgacggggcggagctggaccgggtcgacgtgctccagcccgcctcgtgggcggtgatggtggcgctggccgcgctgtgggagtcgtgcggggtgcgcccggacgccgtggtcgggcactcgcagggcgaggtggccgccgcgtgcgccgccgggtggctgtcgctggacgacgcggccagggtggtggcgctgcgcagccgcgcgatcgccgagcacctggccgggcgcggcggcatgatgtccgtcgccgccggggcggagcgggtggccgggctgatcgccgaccggcagggccgggtgtcggtggccgccgtgaacgggccgtccgcgaccgtggtggccggggccgccgacgcgctgcccgagctggccgcgcgctgcgagcgggagggcgtgcgggcccggatcatcccggtggactacgccagccacaccgagcacgtggacgcgctcgacggggtgctgcaggaggtgctggcgggcgtcaccgcgcaggccgggcacgtgccgtggctgtccaccgtggacggcgagtgggtcgacggctcggggctggacgcggactactggttccggaacctgcgcgggaccgtgcggttcgccgacgcggtggcggcgctggcgggctccgggcaccgggtgttcgtggaggtgtccagccacccggtgctcaccgccgcgaccggcgaggtgctggaggccgccggggtgcgcgacgcgctggtggtcggctcgctgcggcgcgacgacggtggccccgagcggttcctcaccgggctcgccgagctgcacgcgcgcggcgtcccggtggggctggaggcggtgttcgcgggcgcggacgggcgggtggagctgccgacgtacgcgttccagcacgagcggtactggctggcgcgcggcccggtggccggggacgtgtccgggtcggggctggtggacgcggcgcacccgctgctcggggcggtcgtgccgctgccgggcacgggcggggtgctgctgtccgggcggctctcgcaccggcggcagccgtggctggccgagcacgcggtggccgggacggtgctgctgccgggcgcggcgatcgtggagctggccgtgcgcgcgggcgacgagaccgggtgcggggtgctgcgggagctggtgatcgggcagccgctggtggtgccgccggacgccgaggtggacctgcaggtgctcgtcggcggcccggacgacgggggcgtgcgggacctgcggctgtactcgcggaccggggcggcggcggagtgggtcgagcacgcggcaggcgcgctcgcccccggcggcgcggtcggcggggcgcgaccggccggggcgcggacggccggggcgcgactggacggggcgcgactggacggacagtggccacccgcgggcgcggaacccgttgcgctggaaggcttctacgagaacctggcggagctgggctacgagtacgggccgctgttccgggggctcgcggcggcgtggacgcgcgacggcgaggtgttcgccgaggccgtgctgcccgaggaggcgttgtccgggcaggcgttgtccgggcaggcggggtccgggcaggcggggtccgggaacgggtccgggaacgggttcggcatccacccggccctgctggacggggcgctgcacgcgggcaacctgtgcgtgccgcccgcgccgggccggacgctgctgccgttcgcgtggaacgaggtgcggctgcacgccaccggggcgacggcggtgcgggtgcgcgtgcgggcgaccggcgaggactccctggagctggagctgttcgacgccgacggcgcgcccgtggcgagcgtcggcgggctgaccctgcgaccggcggtcacgggcgcgcgcccggccgagtcgctgcacgaggtggagtggaccgaggtcgcggcgggcggttcgtggccggaggtcgccgacacccgcgactgggaggccgccgccgacctgccgacccggtcgcgcgagctggccgcccgcgcgctggaactggtgcaggaccggctggcgggcgtggacggcgcaccgctgctggtgatcaccacgggcgcggtggcggtggccgacgacgccgaggtcaccgacccggccgccgccgccgtctgggggctgctgcgctcggcgcagtccgagcaccccggccggttcgcgctggtcgacgtcgacggcggcgcggcggccgaggtcgccgcgctcgtgcccggcgacgagccgcagaccgcgctgcgcggcgggctcgtgcgggctccgcgcctgcgccgcctgccccccggtctcgtgccgcccgccggggcgcactggcacctggacgcagtcaccaccggcacgctcgacgggctcgcgctcgtggcctcggaaccggtcccgctgcgggccggggaggtgcggatcgaggtcagggcggccgggcagaacttccgggacgtgctggtggcgctggacggcgtcgcgggccaggagggcatcggcggcgagggctccgggatcgtgaccgaggtcggccccgaggtgaccggattcgccgcgggcgaccgggtgatggggctgttcccgcgctcgttcgggccgctggccgtggccgacgcccgcacggtggtgcgggtgccgcgcggctggtcgttcaccgacgcggcggccgtgccggtcgcgttcctgaccgcgctgcacggactccaggacgtcgccgggctgcgggccggggagacggtgctggtgcacgcggcggcgggcggcgtcgggcaggccgccgtgcagctcgcccaccacttcggcgcgcgcgtgctggccaccgcgcacccggccaagcacagcgtgctgaccgcgctgggcgtgcccgccgagcggctcgcctccagccgcgacctcggctacgcgcggcggttcggcgacgtcgacgtggtgctgaactccctggtcggcgagcacgtcgacgcctcgctgcggctgctgcgcgcgggcggccggttcgtggagatcggcaagaacgacgtccgggacgccgactcggtcggggacgtccgctaccgggtgttcgacctgggcgcggacgccgggccggaccggatcggcgagctgctggagcagctggtgggcctgttcgagtcgggcgcgctgcggccactgccggtgcgcacgtgggacgtcacccgcgcggcctcggcgttccgcgagatgagccggggcgggcacaccggcaagatcgtcctgacgatcccgcgccgcctcgaccccgagggcacggtgctgatcaccggcggcgccggcacgctcggggccaccgccgcccgccacctggtcaccgcgcacggcgcgcggaacctgctgctggtcggcaggcggggccccgacgcgcccggcgcgagcgagctggcggaggagctgcgcgggctgggcgcggacgtgcgggtggcggcgtgcgacgtcgccgaccgggccgcgctcgacgccctgctcgcctcggtcccggccgggcgcccgctgacggcggtcgtgcacgcggcgggcgcgctcgacgacggcacggtcaccgcgctcaccccggagcggttcgacgcggtgttccgccccaaggtggacgcgatcgcgcacctggacgaggcgacccgcgacgccgacctggccgcgttcgtcgtctactcctcggcggcgggcgtgctcggcaacgcggggcagggcaactacgcggcggcgaacgccgtgctggacgcggtggcccgcacccggcacgcccgcgccctcccggcgacctcgctggcctgggggttgtggagcgacacgagcgcgctgaccgcgacgatggacgggcgcgcggtggaccgcacgcggcgcgcgggcgtgctgggcatgggcaacgacgaggcgctggcggcgctggacgcgggcctggcgtccgggctgcccgcgctggtggccgcccggatcgacccggccgcgctgcgcgaccccgcgtcggggtcgccgctgctgcgcgggctggtgcgcgccacccgccgcacggccgccacccgcgaccgggacgccgtgggcgggctggccggacggttggccgggttgtcggccgcggagcaggacgagctgctgctgggcctggtgcgcagcgaggccgccgccgtgctcgggcacgcgagcgccgagcgggtcgagccgcaggtggcgttccgggacatggggttcgactcgctcaccgccgtggagctgcgcaaccggctcgcggcggcgaccgggctgcggctgcccgcgacggcgacgttcgaccacccgacgccggtgcggttcgccgcgctgctgcggggcgagctgctgggcgccgtcgtggctcccggagccgtgaccgccgccgcggctcccgtgaccgccgccgcgcccgccgacgagccgatcgcgatcgtgtcgatggcgtgccggctgcccggcggggtggtcgacccggccgggctgtgggagctgctcaccggggagcgggacgggatcgtggacttccccgacgaccggggctgggacctggagtcgctctaccacccggacgccgactcccccggcacctcctacgtgctgcgcggcgggttcctggacgacgcgggcgggttcgacgccgggttcttcggcatctccccgcgcgaggccctggcgatggacccgcagcagcgggtgttcctggagacctgctgggaggcgttcgagcgcgccgggatcgacccggtctcggtgcgcggcagcgacaccggggtgttcgccgggatcatcgaccaggactacggggtgcgcgcgggcacggcccccgaggagctggagggctacctgctcaccggcaccgccacgtcggtggcgtccgggcgggtggcctacctgttcgggctggagggcccggcggtcaccgtggacacggcgtgctcgtcgtcgctggtggccacgcactgggcggtgcaggcgctgcgccggggcgagtgctcgatggcgctggcgggcggcgcgaccgtgatggggcggccgtcggcgttcgtggagttctcccggcagcgcgggctggcgcgggacgggaactgcaaggcgttcggcgcggacgcggacggcaccgcgttcagcgagggcgcgggcgtgctgctgctggagcggctctcggacgcgcggcggcgcgggcacccggtgctcgcggtgatccgggggtcggcgctgaaccaggacggggcgtcgaacgggctgaccgcgcccagcggaccggcgcagcagcgggtgatccgggcggcgctggccgacgcgggcctgcggccgtcggacgtggacgcggtggaggcgcacggcaccggcaccgcgctcggcgacccgatcgaggcgggcgcgctgctggcgacctacggcgcggaccgggagggcgcggaaccggtgtggctggggtcgctcaagtccaacaccgggcacacgctggcggcggcgggcgtgtcgagcgtgatcaagatggtgctggcgctgaaccacggcctgctgccccggtcgctgcacgtgcgggagccgagcgcggcggtggactgggagtcgggcggcgtgcgcctgctgacgagcgcccggccgtggccggagagcggcaggccccggcgggcgggggtgtcgtcgttcgggatcagcggcacgaacgcccacctggtgctggaagccgcgcctgcggaggagggcgcgggggcgcggagtggggcggcggcgccgggaccggacacccggtcggcgcccaccccggacgccccagcgggccccgtccagacctccggcgtgatcccctggccgttgtcggcccgctccgccgacgcactgcccgcgcaggccgcgaagctggccgcccacgtgcgggcgcacgacgacctctcgccgctcgacgtcggctggtccctcgcgaccacccgcaccgcgcacccgcaccgcgccgtgctcgtcggcggcacccgcgaggcgctgctgtcggccgccgacgcgctcgcgggcggcgaggccagccaggccgtgctcaccggctccgccgtcgggtcgggttcggcgaagaccgtgttcgtgttccccggccagggcgcgcagtgggcgggcatgggccgtgagctgctggggtcctcgccggtgttcgccgcgcggctgcgcgagtgcgccgacgcgctggccccgcacaccgactgggacctcctggacgtggtgcgcggcgcggagggcgcgccggggttcgagcgggtcgacgtgctccagcccacctcgtgggcggtgatggtggcgctggccgcgctgtggcgctcgtgcggggtggagccgtccgccgtcgtcgggcactcgcagggcgaggtggccgccgccgtggtcggcgggtacctggcgctgggcgacgcggcgcggctgatcgcgcggcgcagcagggccatcgcgcaggagctgaccgggcgcggcgggatgctgtccgtgctcacctcgcccgagcgggtcgccgaactgctggagccgtgggccgggaagctgtggatcgcggcggtcaacagccccgcgtccgtctcggtgtccggtgacgccgaggcgctgggcgagttcgtgcgggtgctggccaaggcccggatcaaccggtggcggctgcccggcgtggacttcgccgggcactccgggcacgtcgacggcatcgaggcgcggctgcgcgaggagctggccgacgtcaccgccgcggcgggcgaagtgccctggctgtccaccgtggacgggcggtgggtggagcgcaccaggctggacgccgactactggtaccgcaacctgcgcgacgtggtccgcttcgacgaggccgtccgcgcgctggtggacgccgggcaccgggcgttcgtggaggtctccacgcacccggtgctgaccaccgcgatcggcgaggtcgccgacgagcggcaggacgtgcgggtcgccgtggcgggcacgctgcgccgcgacgacggcggcgcggaccgggtcgtgggcgcgctcggcgaggtggccgcctcgggcgtggcggtggactgggcggcggtgttcggcgggaccggggccgcggtggtggagctgccgacgtacgcgttccggcacgagcggttctggctcaccccgtccggcggcgacgtgcgcgcggtggggctgcggcaggccgggcacccgctgctgggcgcggtggtcagcgtcccggacaccggcggcgtgctgctgaccgggcggctgtcgctgtccgcgcagccgtggctggccgaccacgcgctgtccggcgtgccgctgctgccggggacggcgctggtggagctggcggtgcgcgcgggtgacgagaccggcacgccggtggtggcggagctggtgctgggcaggccgctcgtgctgccgcgcaccgggtcggcgcaggtgcaggtgctggtgggcgaggaggcgcgggacgggcggcggccggtcgcggtgtactcgcgggcgggcgacgaccggccgtggaccgagcacgcctcgggctcgctcgcgccggacgaggacgccgcgccgggagcggagggcgacgagtggccgcccgccggggccgagccggtggacctcggcggcttctacgacggcctcgccgaacggggctacgactacggcccggccttccggggcctggtgcgcggctgggtcaggggcgacgaggcgttcgccgaggtcgggctgcccgacgaccagcacggcgcggcggcccggttcgggctgcacccggcgctgctggacgcggccctgcacgcggcctcgctgtgcgcgggccacggccggggcacggcgctgccgttcacctggaccggcgtgcggctgcacgcggccggggcgacggcgctgcgcgtgcggctggaggcggacgggccggagcggttgtcgctgcgggcgagcgatccggcgggcacgcccgtggtgaccgtcgggtcgctgctgctgcgcgccgccgacgcggaccggctgcgggcgacagcggcggcgacggcggcagcggcggcggacgacgggctgcacgcgctggagtggaccccgcacccgctgcccgaggagacgaccggttcccccgccgtcctggacaccagggcgtgggagctgcccgagggcgtcgggcgggccgaggcgatcaccacgcgggtgctcgccgagctccaggccgagctcgacgggacggcgaccctggtcgtggtgacgcggggcgcggtggccgtgcatgacgacgccgaggtcaccgacccggccgccgccgcggtgtgggggctggtgcgcgccgcgcaggccgaggaacccggacgcgtcgccgtggtcgacgtcgacgacgcctccgaggccgcgctggacgccgccgcgcacgccgcgggcgcagaaccgcagctcgccctgcgcggcggggcggcgttcgcgccgaggctggtcgaggcgtccggggcgctggccgtgccggacgggccgtggcggctcgacagcaccggccggggcaccctggagaacctggcgctcgtgcccaaccccgccgccggggcgccgctcgcgcccggtcaggtgcggatcgtggtgcgggcgggcggcctgaacttccgggacgtgctgatcgcgctcgacgcctacgagtcggagatcggcaccgagggcgcgggcgtggtcgtggaggtcgcgccggacgtcacccgcgtggccgtgggcgaccgcgtgatgggcatgatccccggctcgttcgggccgctggccgtggccgacgcccgcacggtggtgcggatgccgcgcggctggtcgttcaccgacgcggcgggcgtgccggtcgcgttcctgaccgccctgtacgggctgcgcgacctcggcggcctggcggagggcgagaccgtgctggtgcacgcggcggcgggcggcgtcggcatggccgccgtgcagctcgcccggcacttcggcgcgcgcgtgctgggcaccgcgcacccggccaagcacgccgcgctggacctgcccgccgaccacctggcctccagccgggacctcgcctacgcgcagcggttcggcgacgtcgacgtggtgctgaactccctggtcggcgagcacgtcgacgcctcgctgcggctgctgcgcgcgggcggccggttcgtggagatgggccgggcggacctgcgcgacgccgacgaggtggcgcgcgagcaccccggccgcgcctacctcccgttcgacctcggcggcgacgccgggccggaccggatcgccgagctgctggtggagctggtggccctgttcgagtcgggcgcgctccgcccgctgccgacccggcgcaccgacctggtgcgcgcccccgaggcgttccgggccatgagccagggccgccacgtcggcaagctcgtgctcaccccgccccgcgcgctcgaccgcgacggcacggtcctgatcaccggcggcacgggaaccctcggcgcggctctggcccgccacctggtggacgcgcacggcgtccggaacctgctgctggtcagccgcagcggccccaacgcgccgggtgcggccgacctggtcgcggagctggccgagcggggcgcgagggtccgggtggccgcgtgcgacgtggccgagaaggacgcgctcaccgcgctgctcgcctcgatccccaccgggcgcccgctcaccggcgtcgtgcacgcggcgggcgcgctggacgacggggtgctcaccgccctggacgccgaccgggtcgcggcggtgctgcgccccaaggccgacgccgccctgctgctgcacgaggccaccgaggacgccgacctcgcgctgttcgccctgtgctcgtcggtggcgggcgtgctgggcaacgcgggccaggcgaactacgccgccgccaacacctacctggacgcgctggcccagcaccggtcggccgccggtctggccgcgctgtcgctggcctggggccggtgggcgcagaccagcgccctcaccgcagacctgcccgcgcccggcggtcgccgcgacctggtgcgccccatggacaccgcgtccgcgctgcgcctgctcgacgccgcgctccgcaccggacgctcgacggtcgtcgccgccgagctggacgtcacggcggccaccgccgcgaacccggtgctgcgcggcctggtccggcccgcccggcgcgcgctggccacgtccgcgcgggacgagcgcggcgtggcggcggcgctggccgggctgggcgaggccgaccggcgccggttcgtgctggacctggtgcgctcgcacgccgccgtcgtgctgggcctggcgggcaaggaggccgtggacgccgagcgcgcgttcaccgagaccggcttcgactcgctcaccgccgtggagctgcgcaaccggctcgccgccgcgaccgggcttcggctgccctccacgctggtgttcgaccacgccaccccgaccgcgctggccgcgcacctgcgcgccgagctgaccggcgacgacctgccgcaggcgcgggccgtcgccgccacggcgggggcgcgggacgacgacccggtggtgatcgtgtcggcgagctgccgcctccccggcggcgcggactcgccggaggcgctgtgggagctgctggagcggggcagggacgccatcaccccgttcccgcgcgaccggggctgggacctggaggcgctctacgacgccgacccggaccggccgggcaagagctacgtgcgcgacggcgggttcctcgccgacgcggccgggttcgacgccgagttcttcggcatctccccgcgcgaggcgctggccaccgacccgcagcagcggctgctcgccgagacctcctgggagctgttcgaacgcgcgggcatcgccccgacctcggtgcgcggcagcgacgtcggcgtgttcgcgggcgtgatcaaccaggagtacggcgtgcacagcggcacgacccccgccgagctggaggggtacgtgatgaccggctcgaccaccagcatcgcctccggccgggtggcgtacctgctcgggctgaccgggcccgccgtcaccgtggacaccgcgtgctcctcgtcgctggtggcgatccacctggcggcgcaggcgctgcgctcgggcgagtgctcgatggcgatcgcgggcggcgcgacggtgatcgcgaggccgggcgggttcgtctcgttctcccggcagcgcggcgcggcccccgacgggcgctgcaaggcgttcggcgacggcgcggacggcatggcgttcgccgagggcgtcggcctggtgctgctggagcggctctcggacgcgcgccgcaacgggcacccggtgctggcggtcgtgcgcggcacggccctgaaccaggacggcgcgtccaacggcctgaccgcgccgaacgggcccgcgcagcagcgggtgatccggcaggcgctggccaacgccgggctgtcccccgacgaggtggacgcggtcgacgcgcacggcaccggcaccgcactcggcgacccgatcgaggcgcaggcgctgctcgccacctacgggcgggaccgggacccgcggcggccgctgtggctggggtcggtgaagtcgaacatcgggcacacccaggcggcggcgggcatcgcgagcgtgctcaagatggtgctggcgatgcagcggggcgtgctgcccgcgaccctgcacgccgacaccccgacgacgaaggtcgactggtcctcgggcgcggtggcgctgctgtcgcgggcgcggccgtggccggagaccgggaggccgcgccgggcgggcgtgtcctcgttcgggatctccggcaccaacgcgcacgtgctgctggagcaggccccgcaggacgcgcccgccacgccggtcgccccgcggggcgccgggctggtcggggcggtggcctggccggtgtccgggcgcacgcccgccgcgctgcgcgcccaggccgccaggctcgggacgcacctggcgggcgcgcaggccggacccgccgacgtgggctggtcgttggcgggcacgcggacggcgttcgcgcagcgggcggtcgtggtggccgggacggcggagcaggcccgtgacgggctggcggcgctggccgaaggccgctcgtccgcgctcgtgacgaccggtgaggccggggtcgacgggcgcgtggtgttcgtgttccccggccaaggggcgcagtggatcggcatgggcgcggagctgatcgacgcgtcgccggtattcgccgagcggttgcgcgagtgcgcggaggcgctggaaccgttcgtggacttcgacctgatcgaggtgctgcgcggacgcgggtcgctggagcgggtcgacgtggtgcagcccgcgtcgtgggcggtgatggtgtcgctggcagcgctctggcggtcgctgggcgtggaaccggacgccgttgtcgggcactcgcagggcgagatcgcggcggcggcggtcagcggggcgctcagcctgcccgacgccgcagccgtggtcgcgttgcgcagcaaggcgatcgcccaggacctggccgggctcggcggcatgatgtccgtcgccctgcccgccgacgacgtcgacctgagcgggtatcccggacgcctgtgggtcgccgcgcacaacggccccacctcgaccgtggtggccggtgacgtggacgcgctgcgcgagctccacgcccactacgagggcgccgaggtccgggcccggatcatccccgtcgactacgccagccacaccgggcacgtcgacaccatccgcgagcggctcgccgaggcactggcgcacgtgcggccgagggcgggcacgatcccgtggctgtcgaccgcgaccggcgagtggaccaccggtgaggacgccgacgccgactactggttccgcaacctgcgcggcgcggtgggcttccacaccgccatcaccaccctcgccgagcagggccaccgggtgttcgtggaagtctccagccaccccgtgctcaccaccgccatcgaggccacgctcgaaggaaccggacccaccgccgtcaccggaaccctccgccgcgacgacggcggccccgaccgcctcctcaccagcctcgccaccctgcacgtgcgcggcgtccacgtcgactgggacgcggtctacgcgggcagcggcgcgcaccgcacgacgctccccacctacgcgttccagcacgagcgctactggctcaccgagccggacgcgccgcaggccgtcgcggacgccccgttctgggacgccgtggacagcggcgacgtggccgcgctcgccgggtccctgggcgtcgagcccgccgccctggagccggtgctgccggggctgacgagctggcgggcccgcaaccgggacggcgcggccgtggacgactggtcctaccggatcggctgggagcgggtggacgtgcccgccgccccgctgtccgggacgtggctggtcgtggtgcccgaggcactcgccgacgacacctcggtcgccgaggtcgcggcggcgctggccgcgcgcggcgcgacgccgaggatcgtggcggcgggcccggacctgggcccggacctgggtgacgagccggacggggtgctgtcgctgctggcgtgggacgaccgcccggccgggggcggcacgctctcgcgcggcgtcgtggacgcggtcgggctggtgcgggaggcggtgcggcgcggctggtcggccccgctgtggtgcgccacgctcggcgcggtcgccgtcgccgaccccggcgaggtgacggccgagttcgggccgcagctgtggggcacgggcgtcgtgctgggcctggacctgccggacacctggggtggcctggtcgacctgcccgcgcggccggacggggtcgcgctggacctgctgtgcgcggtggtcgcgggcgcgggcgacgaggaccagctggcggtgcgcccggccggggtgttcgcgcggcgcatgacccgacgcccggtcgcgtcggcgcccgcgtggcgaccgcgcgggacggtgctggtcaccggcggcaccggcggcctcggcggctacgtcgcccggtgggcggcggagcggggcgcgcgggacgtggtgctgctctcgcgcggcggcccggacgcgccgggcgcggacgccctggtcgccgacatcacggcggcgggcgcccgctgcgcggtgctggcctgcgacgtcaccgaccgggacgcgctggccgaggtggtcgcgaacctgccggacgggccgctgtcggtggtgcacgccgcgggcgtggcgcgaccgggacggccgctggtggagaccacgccggaggagttcgcggccatcggccggggcaaggtcgcgggcgcccgcctgctggacgagctgctgggcgaccgggagctggacgcgttcgtgctgttctcctccggcgcggcggcctggggcagcggcgggcaggccgggtacgcggcgggcaacgccttcctggacgggctcgcgcagcgcaggcgcgcccgagggctcgcggccacctcggtggcctggggcgcgtggggcggcgtcggcacggtcgacgaggtgctgggcgagcagtggcggcgcgccgggctgctcaccatggacccgcgcctggccaccctcgccctcgcgcacgccgtgggctcgggcgaggcgcacctgctcgtcgcggacgtcgactgggcccgcttcgcccccgcctacgcgctggccaggccgcgcccgctgctggcggcgctgcccgaggtcgccgacgcgctggcggtcgtggacgcgcccgccgacgccggggggatcggggcgcggctggccgggctgccgcccgccgagcaggagcggctgctcaccgagctggtgcaggcggaggcggcggccgtgctgggcctgggcggcatcaccggcgaccgggcgttccgggaggtcgggttcgactcgctcacggccgtggagctgcgcaaccggctcggcgcggccacgggtctcaccctgcccgcgacgctggtgttcgaccacccgcgcccgagcgccctggccgcgcacctgcggtccgcgctgggcccggccgccgcgccggtggactcggtggcgggcgtgctggccgagctggaccggctggaggcggccatcccggcgctgccgtcggccgagatcggccggtcccggctggagctgcggctgcggcggttgagcgcccgcgtcggcgagctggtcgccgcgaacggcgagcgggcgaacggcgggcgcgcgaacggcgggcgcgcggcggccgacgagctggacgacgcgggggccgaggacgtgctcgcgttcatcgaccgggagttcggggacgcgtgagcggccacacgagccccgaccccggcccccaccgcggcccccacaacgacgaccctggcgaggaacagatggcgaacgacgagaggctcctcagctacctcaagcgggtcaccgccgacctgcaccgcacgcgggagcggctgcgcgaggcggagtccggggcggacgagccgatcgcgatcgtcggcatggcctgccgcttccccggcggcgtgcgcaccccggacgagctgtgggagctggtggcgtccggccgcgacggcatcggcccgttcccggacgaccggggctgggacctgggcgcgctgttcgacccggaccccgacgccaccggccgctcctacgtcaccgagggcgggttcctggacgacgcggccctgttcgacgcgggcttcttcgggatctccccgcgcgaggcgctggccaccgacccgcagcagcgggtgctgctggagaccgcgtgggagaccttcgagcaggcgggcatcgacccgacctcgctgtccgggcaggacgtgggcgtgttcaccggggtcgccaacggggactacgcgctgaccgtggaccgggtgccggagggcttcgagggctacctgggcatcggcggggcgggcagcatcgcctccgggcgcatctcgtactccctgggtctggagggtccggccgtcacgctggacaccggctgctcgtcgtcgctggtcgcgatgcactgggccgggcacgcgctgcgggcgcgggagtgctcgctggcgctcgcgggcggcgtgatggtgatggcgacgccgggtgggttcgtcgggttctcccggcagcgcgggctggcccgcgacgggcggtgcaagtcgttcggcgacggcgcggacggcacgtcgtggtcggagggcgtgggtctgctgctgctggagcggctgtcggacgcgcgggccaacgggcacgaggtgcttgcggtggtgcgcgggtcggcgatcaaccaggacggggcgtccaacgggctcaccgcgcccaacgggccgtcgcagcagcgggtgatccgcgcggcgctcgacgcggcggggctcgggcacgcggacgtcgacgcggtggaggcgcacggcaccgccacggtgctcggcgacccgatcgaggcgcaggcgctgctgaacacctacgggcggcaccgggacggggcgcagccgctctacctggggtcggtcaagtccaacctcgggcacacccaggcggcggcgggcgtggccggggtgatcaaggcggtgcaggcgatgcgccacggcgtgctgccgcccaccctcaacgtcggcacgcccaccaccaaggtcgactggtcctcgggcgcggtggaggtgctggcggaggcccggccgtggccggagaccgggcgtccgcgccgggtgggcgtgtcgtcgttcggcgtgagcggcaccaacgcgcacgtgatcctggagcaggcacccgagcacgagccagcgccggaggagccgggtgggcgcgggccggtggcggcgggcggcgcgacgccgtggacgctgtccgggcgcacgcccgccgcgctcgccgaccaggcgcggcggctggccgggcacgtgacggccgacctgcgggcggaggacgtcgggttctcgctggccaccaccagggcgcacctggagcaccgggcggtggtggtcggctcggacgggctggcggcgctggccgaaggccgctcgtccgcgctcgtgacgaccggtgaggccggggtcgacgggcgcgtggtgttcgtgttccccggccaaggggcgcagtggatcggcatgggcgcggagctgatcgacgcgtcgccggtattcgccgagcggttgcgcgagtgcgcggaggcgctggaaccgttcgtggacttcgacctgatcgaggtgctgcgcggacgcgggtcgctggagcgggtcgacgtggtgcagcccgcgtcgtgggcggtgatggtgtcgctggcagcgctctggcggtcgctgggcgtggaaccggacgccgttgtcgggcactcgcagggcgagatcgcggcggcggcggtcagcggggcgctcagcctgcccgacgccgcagccgtggtcgcgttgcgcagcaaggcgatcgcccaggacctggccgggctcggcggcatgatgtccgtcgccctgcccgccgacgacgtcgacctgagcgggtatcccggacgcctgtgggtcgccgcgcacaacggccccacctcgaccgtggtggccggtgacgtggacgcgctgcgcgagctccacgcccactacgagggcgccgaggtccgggcccggatcatccccgtcgactacgccagccacaccgggcacgtcgacaccatccgcgagcggctcgccgaggcactggcgcacgtgcggccgagggcgggcacgatcccgtggctgtcgaccgcgaccggcgagtggaccaccggtgaggacgccgacgccgactactggttccgcaacctgcgcggcgcggtgggcttccacaccgccatcaccaccctcgccgagcagggccaccgggtgttcgtggaagtctccagccaccccgtgctcaccaccgccatcgaggccacgctcgaaggaaccggacccaccgccgtcaccggaaccctccgccgcgacgacggcggccccgaccgcctcctcaccagcctcgccaccctgcacgtgcgcggcgtccacgtcgactggaaggccgtgttcgccggcacgggcgcgcgccgcgtcccgctgccgacctacgcgttccagcacgagcgctactggctggaccggggcgcggcggccggtgacgtcacgggcgcgggcctggccgacgcggcgcacccgctgctggccgccgtcgcccagctgcccggcaccggcggggtgctgctgagcgggcggttgtcgcgggcgacgcacccgtggctggccgagcacgtggtgaacgggaccgcgctggtgcccggcacggccctggtggagctggcgctgcgcgcgggcgacgaggtggacgcgcccgtgctgcgcgagctggtgatcacccggccgatgccggtgccggagcggggtttcctgcacgtgcaggtggacgtcgcgggtgcggcggacgacgggtcgcgggcggtgcggatctggtcgcgcgccgaggacgcggcgagcgagacggcccgctggaccgagcacgccaccggctccctcgcccccgacgacgcggccccgcccgcccgcgcgagcggcgcgtggccgcccgagggcgcggcggccgtggacgtggacgacttctacgaccgcctcgcgggcgcgggctacgagtacgggccgctgttccagggcctcaccgccgcgtgggccggggacgggcaggcgtgggccgaggtggtgctgcccggtgaggcgggcgggttcggcgcgcacccggcgctgctggacgcggcgctgcacgtgggcacgttctgcctgcccggcgggccggggtcgcgcacgctgctgccgttcgcgtggacgggcgtgcggctgcacgccaccggcgcgacggcggtgcgggtgcacgcccgcgccaccggcgacgacggcctcgtcgtggagctgcgcgacgcggacggggaaccggtcgtgacggtcgacgcgctcgtgctgcgcgacgccgaccccgccgacgcgcaggccccggacgtcacggcggacgcgttgtggcgggtgcggtgggtcgagcagccgcccgcgcccgcggcgcccggctgggtgctgctgggcgggccgtccgggcacgccgggttcgccgccctgccggtgttcgccgaccctgcggccgtggcggcgctgcccgagggcgaccggcccgcggtggtcgtcgtggacaccaccgcgtgtcgggagccggggcgggacgtgccgggggcggcgcgggcgttcgtggtgcgggcgctggagctgctggtggcgtggctgcgcgaggacgcgctggccgggacccgactggtgctagtcaccagcggcgcggtgccggtgcgcgcggacgccgaggtcaccgaccccgctgccgcggcggtgtggggtctgccgcgctcggcgcagtcggagcacccggaccgggtgtggctgctggacgtcgacgagccgggcgcggcgccgggcgcgctggcctcgccggagccgcagctggccgtccgggcgggcgcggggttcgcgccccggctcgccagggccgaggccgcgcccggcgcgctgccggtcgacgggccggtgctggtcaccggcggcaccggcacgctcggcgcgctcgtggcccggcacctggtcaccgcgcacggcgcgcggaacctgcacctggtgagcaggcgcggcccggacgcgtccggcgctcgagaactcctggacgagctgcgcgggctgggtgccgaggtcgagctgtcggcgtgcgacgtggccgaccgggtggcgctcgccgccctgctggggcgcgtgcgccccgccgccgtggtgcacgcggcgggcgcggtggacgacggcctgctcaccgacctgaccgccgaccggttcgacgccgtgctgcggcccaaggtcgacgcggtcgcccacctggacgacctactcggggacgtgccgctggtggtgttctcctccgcgaccggcaccctcggcacccccggccaggcgaactacgccgcggccaacgcggtcgccgacgcgctcgtgcagcgccgccgcgcgcggggcctgccgggcgtgtcgctggcgtggggcctgtggtcggacaccagcgagctgaccgcgaccatggacgccgccgacgtggcccgcacccgccggggcggggtgctcggcctggacgcggcgcgcggcctggcgctgctcgacgccgcgctcggcgcggacgacgcgctgctcgtgccgatccacctggacaccgccgcgctgcgccggggggccgacccggctccgccgctgctgcgcggcctggtccgccgcgcccggcgcgcggcgggcgcggcccggcaggccgcgctgccgctggtggcgcgactggccggggtggacgcggcggagcggcggcgggcgctggtggagctggtgcgcgccgaggccgccgccgtcctcgggcacggcggcccggacggcatcgggcaggaccagccgttccgggaggtcgggttcgactcgctcacggccgtggagctgcgcaaccggctcggcgcggccacgggtctcgcgctgcccgcgacggtggtgttcgaccacccgacgtccgcgcgggtcgccgagcacctgcgggagctgctgttcggcgcggagacggctcaggcccccgcgcggcgggaggtggtggccgacgacgacccgatcgccgtggtgggcatggcctgccggttccccggcggggtcgccgacgcggacgggctgtggcggctggtcgccgaggagcgcgacggcatcggcccgttcccggacgaccggggctgggacctggcggcgctgttcgacccggaccccgaccacgcgggcacctcgtacgtgcgggaaggcgggttcctcgacggggcgaccgggttcgacgcgccgttcttcggcatctccccgcgcgaggcgctggccatggacccgcagcagcggctgctgctggaggtggcgtgggagacgttcgagcaggcgggcatcaacccgcgctcggcgcacggcaccgacaccggggtgttcgcgggcgtgatctaccacgactacggcgaggcggcgggcgagctgcccgagggcgcggagacctaccgcagcaccggcacgtcgggcagcgtggtgtccggccgcgtcgcctacgcgctgggcctgaccggcccggcgctgaccatcgacacggcctgctcgtcgtcgctggtggcgatccacctgggcgcgcgggcgctgcgggcgggcgagtgctcgatggcgctggtcggcggggtgacggtgatgtcgacgccgggcgggttcgtgagcttctcgcggcagcgcgggctggccccggacgggcggtgcaagtcgttctcggagggcgcggacggcaccgggttcagcgagggcgtcgggctggtgctgctggagcggctgtcggacgcgcgggccaacgggcacgaggtgcttgcggtggtgcgcgggtcggcggtgaaccaggacggggcgtccaacgggctcaccgcgcccaacgggccgtcgcagcagcgggtgatccgcgcggcgctcgacgcggcggggttggggcacgcggacgtggacgcggtggaggcgcacggcacgggcaccaccctcggtgacccgatcgaggcgcaggccgtgctcgccacctacgggcaggaccgcgagcagccgctgtggctgggcacgctcaagtccaacctcgggcacacccaggcggcggcgggcatcggcagcgtgatcaagatgatccaggcgatgcggcacggcgtgctgccgcgcaccctgcacgtcaccgagccgaccacggccgtggactggggcgcgggcgcggtggagctgctgacgcgggcgcgggagtggccggagaccgggcgtccgcgccgggcgggggtgtcgtcgttcggggtgagcggcacgaacgcgcacgtgatcctggagcaggcccccgaaccggtggcggtggaggcggcgccggaggcgggggtgctgccgtgggtgctgtcggcccgcacgcccgaggcgctgcgggagcaggccgaccggctcgtggcgcacctgggcggtgagtcgtcctcggcggccgtggcccggtcgctggtgctgggtcgggcggccctggaggagcgggccgtggtcgtgggcgaccgggcgcgcgccggggaggcgttgcgggcgctggccgaggggcggccctcccccgcgctcgtcaccgggcggaccggggtcgaggggcgcgtggtgttcgtgttccccggtcagggcgcgcagtgggtcggcatggggcgtgcgctgctggacgcctcgccggtgttcgccgaacgcctgcgcgagtgcgcggcggccctgcgcccgtacaccgactgggacctggtcgaggtgatcacctcgggtggcgcgctggacgacgtggacgtcgtgcagcccgcgtcgtgggcggtgatggtgtccctcgcggcgctgtggcgctcgctcggcgtcgaaccggacgcggtgatcgggcactcgcagggcgagatcgccgccgcgaccgtcgcgggctggctcagcctccaggacggcgcgaagatcgtcgcgctgcgcagccagctgatcgacgaggagctgaccgggctgggcggcatgatgtccgtcgccctgcccgccgaggacatcgacctgagcggttacgagggccggttgtgggtcgcgacggtcaacgggccgagcgcgaccgtggtcgccggggacaccggggcactggaggagctgcggcgcggctgcggcgaggcggtccgcacgcgggtgatccccgtggactacgccagccacaccgggcacgtcgacgccatccgcgaccagctcgcccggatgctcgccgacgtcaccccgcggcccggcgagatcccgtggctgtccacggtgaccggcgagtggatcacccccggcgacgacgacgccgactactggttccacaacctccgccgcaccgtccacttcgccgacgggatcaccaccctgctcgacgccgggcaccgggtgttcgtggaggtctcctcgcaccccgtgctggcggcggcggtgcaggagagcgccgaggcggccggggacgcgcgggtcgccgtgaccggcacgctgcgccgcgacgacggcggctgggaccgggtcctgaccggcctggccgagctgcacgtgcgcggcgtggacgtggactggacgcgggtgctgcccgaggcggggcgggcgccgctgccgacgtacgcgttccagcacgagcgctactggccggaacccgcgcgcccggccgccgcgccgggcggtggtgacgacgcgctgtgggcggtgatcgagggtggtgacgcggcggacctggccggggagctggccgtggacgaggacgagctggcgcgggtgctgcccgccctgacctcctggcggcggcgcagccgggcaaggagcgcgctcgacggctggcgctaccgggtcgactgggtcccggtccccacgagcgggtctgggctgcccggcgggcaagcgctgtccggcgggcaggcgctgtccggcgggccgaggtcgtccggcggggcagggctctccggcggtcaggggacgccaggcgggtccgggtcgcccggcggagcggcactgccaggcgggccagggtcgcccggcggagcggcgctgcccggccgggtggccgtggtggtgcccgcgaacgacgagcgggcgcgggcggtcgcgggcgggctggtcgcgcggggtgtggacgtgaccgtcgtggcggcggtcgacgccacccgcgacgggctggcgaaggcgctgcccgaccgccccgacgccgtggtgtcgctgctgtcctgggacgcgggggccgacgagccgggcgcgcccggttcggccacggccgcgctggtgcaggccctggccgaccggggtgccaccgggccgctgtggtgcgcgaccgggggcgcggtgagcgtcgcgggcgaggacgccgaccccgaccaggccgccgtgtgggggttgggcggggtgctggccctggacctgccggaggcgttcggcggactggtcgacctgccgcggcagcccaccgacgccgacctcgacgcgttcgccgccgcgctgaccgcccccggcggcgaggaccagctcgcggtgcgcgacggccgcctgctggcccgccgcctggtccgcgacggggccgacgcgccggagtggacgccgcgcggcgcggtgctggtcaccggcggcaccggcggcctcggcacgcacgtggcccgctggctcgcccgctccggggccgggcacgtcgtgctcgccagccgctccggccccgccgcccccggcgcggccgggctggccgccgaggtggaggcgctgggcgcgcggtgcagcgtggtggccctggacgtggccgaccgggacgcggtggccgccgtgctcgccgacgtcgagcgggacgggccgctgaccgccgtggtgcacgcggcgggcgcgggactggccccgacgccggtggtggagctgaccgccgggcggtacgcggacgtcgcggccggcaaggtcgagggcgcgcgggtgctggacgaggtgctcgccgaccgggcgctggacgcgttcgtgctgttctcctccggcgcggccgtgtggggcagcggcgggcaggccccgtacgcggcggccaacgcgttcctggacgggctggccgcccgcaggcgggcgcgcgggctcgtggccacctcggtggcgtggggcggctggggcggcggcctcggcatgatcggcgacggggacgcggagcggtgggcccggctgggcatccgcacgatggacccggaggcggcgctgcgcggcatggcgctggcggtcggctccgggcgggccgcgagcgtggtggccgacgtcgactgggcccggttcgcccccggctacgccctggcgcgggagcgcccgctgctgcgcgggctgccggaggtggtggcgctgctggccgaaccggacgagcccgccgcggcggtggacgcgcggggcgcgctggcggcccggctgaccgggctggacgcggccgggcaggacgagctgctcgcggacctggtgcgggcgcaggcggcggcggtgctggggttcgccgaccctggcgcggtcgcggcggaccgggcgttcaaggacgccgggttcgactcggtgaccgccgtggagctgcggaaccggctgggcgcggccaccgggctgcggctgccgccgaccgtggtgttcgaccacccgaaacccctggctctggcgcgcgtgctgcgcgccgagctggtcccccagcggggggacggggtgacggcggcgcaggtggcgcaccgggaggacgcgatccggcgggtgctggcgtcggtgccgctggcccggttcgaggagctgggcgtgctcggcgcgctcgtggacctcgtgcccgccgcgccaccggcgggcggcgcggcgacagcggagcgggacgacctggcggacctggcggagctggacctggacggtctggtccgcagggcgatgcgcggcaccaccgccgggaacgactgaggctttgatgcggagcggagagagcatgagcgcgggcacctcgccggagagcgtcgtccaggccctgcggaccacgctggtggacaacgagcggctgcggcgggagaacgagcggctggtcgccgaggccggtgagccggtggcgatcgtgtcgatggcgtgccggctgcccggcggcgtcaccgacccggagtcgctgtgggagctggtgcgcgagggccgggacgccatcgggccgttcccgaccgaccggggctgggacctggggtcgctgttcgacgacgacccggacgcggcgggctcctcgtacgtgcgggagggcgggttcctggcgcgggcgggcgggttcgacgcgccgttcttcggcatctccccgcgcgaggccctggccatggacccgcagcagcggctgctgctggaggtggcgtgggaggccgtggagcgggccgggctcgacccgcgctcgctggagggccgggacgtcgcggtgttcgcgggcggcaacccgcagggctacggcggcggaccgggtgacgccccggagggcctggaggggttcctgggcgtcaacgcctcgtcgtcggtgatctccgggcgggtctcctacaccctgggcctgaccggcccggccgtcaccgtggacacggcgtgctcgtcgtcgctggtggcgatccacctggcggtgcggtcgctgcgctcgcgggagtgctcgatggcgctcgcgggcggggtgaccgtgatggggcagccgaccgcgttcgtggagttctcgcggcagcgcgggctcgccccggacgggcggtgcaagtcgttcggcgacggcgcggacggcacgtcgtgggccgagggcgtcggcgtgctgctgctggagcggctctcggacgcgcggcgcgacgggcacgaggtgctggcggtgatccccggctcggcggtgaaccaggacggggcgtccaatggcctgaccgcgccgaacggcccgtcccaggagcgggtgatcgcggcggccctggccgacgccggtctcggcctggccgacgtggacctgctggaggcgcacggcaccggcaccaggctgggcgacccgatcgaggcgcgggcgctgctcaacacctacggccggggcaggccgcaggaccggccgctgtggctggggtcggtgaagtcgaacctcgggcacgcccagtcggcgtcgggggtggcgggcgtgatcaaggtggtgcaggcgatccggcacggcctgatgccgcgcacgctgcacgccgacgagccgagctcgaacgtggactgggcggcgggggcggtggagctgctggcgcgcgagcgggagtggccggagaccgggcgggcgcggcggggcgcggtgtcgtcgttcggggtgagcggcacgaacgcgcacgtgatcgtggagcaggcgcccgaggaggccgccgccggggtcgcggcggcggggcggcccgcgcccaggtcggcgggcgggcaggacgccgggatcgcggcggtgaccgggcaggccgcccccgccgctggccccgccaccgccgaacccgccgcgtcggccgtcgaggacgggaccggcgtcgcccccggcccggtcgcgaccggcggggtcgtgccgtgggcgctgtccgggcggaccgccgccgcgctggccgcccaggcggcccggttgcgcgcgcacctcgccgcgcacccggcggcccgcccggtggacgtggcctggtcgctggccacgacccgctcggtgctggagcaccgggccgtcgtgcccgccgcctcgctcgacgaggccctggcggggttggacgcgctcgcctcgggccgcgcggaccggtcggtggtcgtcggcgaggcggcgcccggccgggtggcggtgctgttcaccgggcagggcagtcagcgggccggtgcggggcgcgagctgcgggagcggttcccggtgttcgcgcgggcgttcgacgccgcgtgcgccgccgtgggcgagctgcccaccggcgacggcggcgcgatcgggctcgccgaggtggcgctggccgaccccggcacgcccgccgccgcgctgctcgaccggaccgcgttcacccagcccgccctgttcgcgctggaggtcgcgctgttccggctggtccagtcgtggggcgtgcgcccggcggcgctggccgggcactcggtcggcgagatcgccgccgcgcacgtggccggggtgctctccctcgccgacgccgccgcgctggtgcgcgccaggggcgggctgatgcaggagctgcccgagggcggcgcgatggtggcggtggaggcggccgaggacgaggtcgtgccgctgctcggggacggggtgtcgctggccgccgtcaacggcccgacctcggtggtgctctccggcgacgaggaggccgtcaccgccgtcgccgcgaggctggcgcagcggggcaggcgcaccaagaggctcgccgtctcgcacgccttccactcgcaccgcgtggacccggcgctggccgccttccgcgccgtggccgaggagctcgcctacgccgcccccacgatcccgatcgtctccaccctcaccggccgccccgtcacccccgacgagctgcgctcccccgactactgggtgcggcacgcgcgcggcgccgtccggttcctggacgccgtgcgggcgctgggggacgcgggcgcgcgcacgttcctggagctgggcccggagggcgtgctcacggcggcgggcgcggactgcctgccggacgcggtgttcgcggcgacgctgcgcgccgacgtgcccgaggcgcgggccgtgctcgccggggtcgcgggcctgcacgtgcgcggcgcgacagtcgactggggttcgctgttcacgggcgcggacgcgcggcgcgtcccgctgcccacctacgcgttccagcacgaggaccactggctggtgcgccgctccaccgccgccgacgtgggcgcggtcggcctgcgcgaggccgggcacccgctgctgggcgcggtcgtcgcgctgccggagagcggcggggtgcagctgagcggtcggttgtcggtggcggcgcagccgtggctggccgagcacgtcgtctccggcacggcgctggtgccgggcgcggcgctggtggagctggcggtgcgggcgggcgacgagaccggcacgcccgtgctggaggagctggtgatcggccgcccgatgccgctgccggacggcggcgcgctgagcgtgcaggtcgtcgtcggcccggacgagggcgggcgccggtcggtgcgcgtgtactcccgcgcggacggggcggtggactgggtcgagcacgcggcgggggcgctgaccgcgccggaggccgcgccgaccgccgacgcgggcccgtggccgccggagaacgccgaacccgtggacacgcggggcttctacgacaccctcgcggagggcggctacgcctacggcccgctgttccggggcctgacctcggcgtggcgcggcgagggcgaggcgtgggcggaggtggcgctgcccggtgacgcgaccgggttcggcatccacccggccctgctcgacgccgcgctgcacaccgcgcacttctgcctgcccaccgggaccgagcggcgggccgggctgctgccgttcgcctggaccggcgtgcggctgcacgcgggcggcgcgacgaccgcgcgggtgcacgcccgcgccaccggcgacgacggcgtgaccgtgcgcctgctcgacggtgccggtcagccggtcgcggacgtggccgccctgaccttccgcgccgcagccgacaccccgtccgccgaggtcccggacgcgctgtgggcggtggagtggaccgagcacccgctgcccgcggacgggaccacccccgcgggcgggaccaccacggccgtggtggtcgtggacacccggagcgtcgacgcccccgacgacggccccgcccgcgcccgcgcgctgaccgcccacgtcctcgccgagctgcagcggcacgccgacgacgaccggccggtcgtcgtggtcacctcaggcgcggtcgccgtgcgcgtcgacggcgaggtcaccgaccccgcgtccaccgccgtgtgggggctggtgcgggccgcgcaggtcgagcagcccgaccgggtccggctggtcgacgtcgagccgggggccgacccggtgctcacctcgcccgagccgcaggtggcgctgcgcggcgggaccgcgcacgtgcccaggctggtccgcgcccgccgcgccctcccggcgccgaccgcgacgtcgtggcggctgggctccgaccgccccggcacgctggactccctcgccctgctcccggacgactccggcacggccccgctcgcccccggcgaggtgcggatcgcggtccgcgcggcgggcctgaacttccgcgacgtgctggtcgcgctggggatgtaccccggtcgcgcggtgatcggcgcggagggcgcgggtgtggtcgtggaggtcggccccggccccgacgacaccgacgccggcgacaccggccccggcgacaccggctcgggcggcctggccgtgggcgaccgggtgatgggcctgttccccggcgcgttcggcccgctggccgtggccgaccaccgaatggtgacccggatgccggacggctggtcgttcaccaccggcgccggcgtgcccatcgcgttcctgaccgccctctacgggctgcgcgacctcggcgggctcaccgcgggcgagaccgtgctggtgcacgcggcggcgggcggggtcggcatggccgccgtgcagctcgcgcgggcgttcggcgctcgggtgctgggcaccgcgcacccggccaagcacgcggccgtgacccgcctgggcgtccccgagtcccacctgtcctccagccgcgacaccgcctacgccgacctgttcggcccggtggacgtggtgctgaactcgctcaccggcgagcacgtggacgcctcgctggggctgctgcgcgcgggcggccggttcctggagatgggcaagaccgacctgcgcgacgccgacgaggtcgcgaaggcgcaccccggcgtcgcctaccgcccgttcgacctgggcggcgaggcgcccgccgagcgcgtcgcggagctgctggccgagctggtcgcgctgttcgaggcgggccgcatccacccgctgcccaccgcggcctgggagatcacccgcgcgccggaggcgttcggctggatgagccgggccgggcacgtgggcaagatcgtgctgaccctcccccgccgccccgacccggacggcacggtgctggtcaccggcggcaccggctcgctcggcgcggtcgcggcccggcacctggtcaccgcgcacggagcccgccacctgctgctcgcctcccgacgcggcgagcaggcccccggcgcggcggagctgaccgacgggctgcgcgggctgggcgcggacgtgcgggtcgcggcgtgcgacgtcgccgaccgggacgcgctcgccgcgctgctcgccacgatccccgccgcgcacccgctcaccgccgtcgtgcacacggcgggcgtgctcgacgacggcgtgctcgccgcgcagacccccgagcgcctggacgcggtgttccgccccaaggtcgacgccgtcgcgaacctgcacgagctgaccggcgacccggccctgttcgcggtgtactcctcggcctccggcgtgctcggcggcgcgggccagaccaactacgccgccgcgaacgcctggctcgacggcctcgcccacgtccggcgcgcggcgggcctgcccgcgacctcgctggcctggggcctgtgggcgcaggacggcggcatgacgggcggcctggcgggcggaccggccgggccgggcgggcgggcccgccggggagccgtcgcgccgctgtccaccaccgagggcatggcgctgttcgacgcggccgtcgcgtcgggccgcccgctcctggccccgatcaggctcgaccccgccgcgctcaccgccgacggcgcgcagccgcccgcgctgctgcgcggcctggcccgccccacccgccgcaccgccgtcgcggccaccaccgacgacggcctcgcgggcaggctcgccgcgctcgacggccccggcaggcagcggctgctcgtggagctggtgcgggagcaggccgccgccgtgctgggcttcgcgaccccggacgccgtgtcgccgggccgggcgttccgggacctgggcttcgactcgctgacggccgtggagctgcgcaaccgcctctccgccgccaccggcctgccactgcccgccaccaccgtgttcgaccacccgaccccgctggacgcggcggcccacctgctcgacgcgctgggcgtcgcccccgcgcccgccccggccaccccggtcgtgacggccgcgcgggacgacgacccgatcgcggtcgtcgccatgggctgccgcctgcccggcggcgtgtcctccccggaggacctgtggcggctgctcgacggcggcgtcgacgccatcggcccgttcccggacgaccggggctgggacctggggtcgctgttcgacgacgaccccgacgcggtcggcaagtcctacgtgcgcgagggcgggttcctggcgggcgcgggcgggttcgacgccgcgttcttcggcatctccccccgcgaggcgctcgccatggacccgcagcagcggctgctgctggaggtggcctgggagaccgtcgagcgggccgggatcgacccgacctcgttgcgcggcgcggacgtcggcgtgttcgccggggcgggcgcgcagaactacggcagcggccccggcccggtgcccgagggcctggagggctacctgggcgtgggcggcgcgacgagcgtggtgtccggccgcgtctcctacacgctcggcctcaccgggcccgcgctgacgatcgacaccgcgtgctcctcgtcgctggtggcgatccacctggcggtgcggtcgctgcgctcgggcgagtgctcgatggccctggcgggtggggtcgcggtgatgggcgagcccgcggcgttcgtggagttctcccggcagcgcgggctcgccccggacgggcggtgcaagtcgttcggcgcggaggcggacggcacgacgtgggccgagggcgcgggactggtgctgctggagcggttgtcggtggcgcgggcgcgcgggcacgaggtgctggcggtgctgcgcgggtcggcggtcaaccaggacggggcgtccaacggcctgaccgcgccgaacggcccgtcgcaggagcgggtgatccgggcggccctggccgacgcggggatcaccccggacgcggtggacgcggtggaggcgcacggcaccggcaccaccctcggtgacccgatcgaggcgcaggccgtgctggcgacctacgggcaggaccgcgagcagccgctgtggctggggtcgctgaagtcgaacatcgggcacgcgcaggcggcggcgggcgtcgcgagcgtgatcaagtccgtgctggcgctgggccggggcgtgctgccccgctccctgcacgccagcaccccgaccccgcaggtcgactggtcctcgggggcggtggagctgctggcgcgggcgcgggagtggccggagaccgggcgtccgcgccggatcggggtgtcctcgttcggggtgagcggcaccaacgcgcacgtggtcctggagcaggcccccgagccggaacccgcgcgggaggcggaacccgcgcgggagtccgcgccagggccggagtccgttccgccgctgaccggggccacgccgtggctgctgtccgcccgctcccccgaggcgctggcggaccaggccgcccggctggtggacgccgtgcccgccgagtggcgggcctccgacgtgggctggtcgctggccaccacgcgggccccgctggagcagcgggccgtggtcgtggcgcgggacaccgcgcgcgggctcgccgccgcgtccgcgctggccgccggacgccccgacccgcacgtggtcaccgggaccgccgacgtggacggcaggaccgccttcgtcttccccggccagggcgcgcagtgggcgggcatggggcgggaactcctggacgcctcgccggtgttcgccgaacgcctgcgcgagtgcgcggcggccctgcgcccgtacaccgactgggacctggtcgaggtgatcacctcgggtggcgcgctggaggacgtggacgtcgtgcagcccaccagctgggcgatcatggtgtcgctggccgcgctgtggcgctcgctcggcgtccacccggacgcggtgatcgggcactcgcagggcgagatcgccgccgccaccgtcgcgggctggctcagcctccaggacggcgcgaagatcgtcgcgctgcgcagccagctgatcgacgagcacctgaccgggctcggcggcatgatgtccgtcgccctgcccgccgaggacatcgacctgaccggctaccagggccggttgtgggtggccgcccacaacggccccaccgcgaccgtggtcgccggggacgccgacgccctggcggagctgcgggacgcgctggagggcgaggcccgcacccgcgtgatccccgtcgactacgccagccacaccggccacgtcgacgccatccgcgaccagctcgcccggatgctcgccgacgtcaccccgcggcccggcgagatcccgtggctgtccacggtgaccggcgagtggatcacccccggcgacgacgacgccgactactggttccacaacctccgccgcaccgtccacttcgccgacgggatcaccaccctgctcgacgccgggcaccgggccttcgtcgaggtctccacgcaccccgtgctcaccccggccgtgcaggaggccgccgaggcgaacccggcgctgcgcaccgtcgccgtgggcaccctgcgccgcgcggacggcggcgcggagcgggtggtggcgggcctggccgagctgctggcgcgcggggtggccgtggacccggcggcggtgttccccggtgcgaggcgggtcgcgctgccgacgttcgcgttccggcacgagacgttctggctctcgcgggcgctgcccgacgcgcggccggtgccgcagggcgggcacccgctggccccggtggtggtgagcgatccgggcacgggcggggtgatcctgtccggccggatctccgcggccacccacccgtggctgctcgaccacgccgtcgcgggcgcggtgctgctgcccggcgcggcgctggccgagctggcggtgcgggccggcgacgagaccgggacgcccaccctggaggagctggtgatcggcaggccggtggtgctgcccgaggacggggagctgcggctccaggtggtcgtgggcgccgaggacggggcgcgccgcgaggtgcgcgcctactcccgcgccgacgacgccgcgccgtggaccgagcacgcgagcggcacgctgtcggcgaagtcctcgctgcccgccgacgtcccggccgccccgtggccgcccgcgggcgcggagccgatcgcgctggacgggttctacgaggccatggcaggggccggttacgggtacgggcccgcgttccgggggctgcgcgcggcctggcgcgacggggacgacgtggtcgccgaggtggccgtgccgcgggcgcaggagcaggtggcgggccggttcggcatccacccggcgctgctggacgccgccctgcacgccgggaacttctgcttccccgcgcaggacggcgagcgggccacgatgctgccgttcagctgggacgacgtgcggttgcacgccaccggcgcgacgtcggtgcgggtgcgggcccgcgcggtgggcggccctggcgcgcccgcgctgaccgtggcgatcaccgacccgagcggggtgccggtggccggggtgggcgcgctcgggatgcgcgcggtcagccccgagcagctgggcgcgccgggcgtcggcggtgacgcgctgcgggtgctggagtgggccgaggtggcggtcgaggcggcggaccggtgggccgtgctgggctccgagcggcacccggacgtggacgcctacgcggccgacccggaccggccgggggcgctgctggtggacgtgggcgcctggctgggcggcgacgacgccgtggcccgcgcgcacgcgctgaccagcgcggcgctggagctggtgcgggactgggcgacccgcggggacctgggcggtgagcggctggtgctggtcacgaccggggccgaggacgtgcgcgacaccgcgccccgcgacccggcgcaggccgccgtgtggggcctggcgcgctcggcccgctcggagcacccggaccggttcgcgctggtcgacgcggacgaccggtccccggcgacgctcgccctggcggccgggtcggcgttcccggaggtggtcctgcgcggcgagcgggcgcacgcgccgaggctggcgcgggccgtccccggcaggccggtggcgctggacccggacggcacggccctgatcaccggcggcaccggcgccctgggcgcgctcgccgcccggcacctggtgaccgcgcacggcgtgcggcgcctgctgctcaccggccgccgggggccggacgcccccggcgcggcggagctggccgaggagctgcgcgggctgggcgcggacgtgcgggtggaggcgtgcgacgtcgccgaccgggacgcgctcgccgcgctgctcgcgtcgatccccgccgggcgcccgctcaccgccgtcgtgcacgcggcgggcgcgctcgacgacgccccggtgaccgacctgaccccggagcggctgtccgccgtgctggccccgaaggtcgacgcgctggccaacctggacgagctggtcggggacgggcccgcggtgttcgcggtctactcctcggcgtccggggtgctcggcacggccgggcaggcggcgtacgcggcggccaacaccttcgcggacgcgctggtgcgccgacgccgggccgagggccgggcgggcgtgtcgctggcgtggggcctgtgggcaggcgccagcgagctgaccggcgacctggccggtgaccggctcgcccgcacccgccggggcgggctggtgccgctgaccgccgccgagggcatggcgctgttcgacgcgggcgcggtcaccacgggcggcccggcgctggtcgtgccgctgccgctggacctggcggcgctgcgcgcctccgcgcgcgacgaggcggtgcccgcgctgctgcgcgcgctcgtccccgccgcgcggcgctcgctctcccccgccaccgggcaggccgcgcccccggccgggttgcgggcgcgcctggccgggctgtcgggcgacgagcaggaggccgtgctcaccgagctggtccgcgacctggccgccgccgtgctcgggcacggcgagaagggcgcggtgggcccggacgacgcgttcttcgagatcggcttcgactccatgacggccgtgcagctgcggaaccggctgaacaccgccaccgggctgcgcctgcccgccgcgctgctgttcgaccagccgacgcccgcgatcgccgccgaggcgctgcgcgagcgactggccgccgagcaatcgggctcagggcaatcgggcgcagggcagccgggcgcagggcattcaggcgcagggcagtcgagcgcagggcgatcaggcgcagggcagtccaccgacccgaccgacgagaggtgagcaccagcatgatcgacgtggccgagtacctgcggcgcatcggcgtggagggcggcgtgccgagcccgacgctggagtcgttgcgggcgctgcacaagcggcacctgatgtccgtgccctacgacaacggcggcgcggccgaccggttgccgccgaaccgggggctcgcggagatcccgctgccccgtgtgttcgcgcacgtggtgaccggccgcaacggcggggtctgctacgagctcaaccggctcttccacgccctgctcaccgcgctgggctacgaggtgctgatggtcgcggcggcgatccggctggccgacgaccggttcgggccggacgaggagcactcgttcaacctggtgcgcctggacgggcggacctggctggtggacgtggggttcgtcggcccgtcctacctggagccgctggagctgtcggcggtcgagcaggagcagtacggctgcgcctaccgggtcgtggagcgcggggacgcgcacgtggtggagcgcaggcccagggacggggcgtggcaggcggtgtaccggttccggccggggcgggcggaccgggacggctgggaggcggtgcggttggacgggctggacgactacgcgcgggactcggtgctggcgggcaccacgttccggggtcgggcggcggagaacgggcagcacgtgctgatcggccgccgctacttcaccgtgctggacggggtggagacgacgcgggtgctcgtgaagaaggacgagttcgcccgcgtcaccgagtcgatcatgatcggggggtgagcgcgtggcgggcgaggtcgagcacgacgtggtggtcgtcggctacgggccggtggggcagctgctgtcggtgctgctggcgcagcgcggctggcgggtgctggtgctggagcgctggccgacgccgttccggctgccgcgcgcggtcgggttcgacagcgaggcgacccgcgtgctggcctcggccgggctcgggcccgcgctggccgagttcggggagcccgcgggcgactacgagtggcgcaccgcgtccggggagacgctgatcgcgttcaccgtgcgggaggaggggcactgcggctggcccgaggcgacctcggcctaccagcccgcgctggaggacgcgctgatcgcgcgcggcgaggcgctgccgggggttcaggtgcggcgcggctgggaggtgaccgggctgaccgaccggggcgaccacgtgcgggtggtggccaccgaccccggcggggcgcgcgtgaggctgacggcgcggttcgcggtcggctgcgacggggcgaacagcgtggtgcgggcccgcaccggcaccgacgtgaccgacctggacttctcgcacgactggctggtgtgcgacgtgcggctgcacgaccggcgcccggtgacgccgaacaacctccaggtgtgcgacccggccaggccacgcaccgcggtgtcggcggggccagggcaccggcggtacgagttcatgcgggtgcccggcgacgacccggagcggttcggcacgccggagagggcgtgggagctgctggcgctgttcggcgtcgggcgcggcgacggggtgctggaccggctggccgtgtacacgttccaggcgcggtgggcgcggcggtggcgggcgggccggatgctgctggccggggacgccgcgcacctgatgccgccgttcgccgggcagggcatgacctccgggttccgggacgcggcgaacctggcgtggaagctggacctggtgctgcgcggcgaggccgggtcggcgctgctggacagctacacgctggagcgcgccgagcacgtgcggcacgccgtgacgatctcggtgggcctggggcgggtggtgtgcgtggccgacccggcggtggctgcggaccgggacgcggcgatgctggcggcgcgcgagcgcgagctgacaccgggcgcgtcggcccggtcggtgctcaagcccctggaggacggggtgctgcaccgggacggcgacggcgccctcgcgccgcacgcgggggccgtgggcccgcagtggcgggtggggcgcggcgggcgggtcgggctgttcgacgacgtggtggggaccgggttcgcgctgctcaccaggggcgggctggtggcggggccggaggtgcgggcgcggctggacgggctgggcgcgcgctacgcgcacctggtgcccgccggggcggcggcggacgggccggacgacgtggtcgacgtgagcgggaactacctgacgtggctggaggagctggacgcggcggcggtgctgctgcgaccggacttctacgtgttcggcgcggccggggacgcggcggggttggccgggctggtggcggacctgcgcgcgcggttggggtgacgccccgcaggccccggcacgtgccgcgccggggcctgctcgcgcgtcacgtccggtcgtcggcgaggtgggccaggcaccagtcgagcacctgcgagggcttgcggaccaccgcgtccgggttcgccgccagcagctccgcctcgtccgtctcgccccacagcgcggccagcgccgggtagcccgcggcgcgggcgctggccaggtccgtcagggcgtcgcccaccatcaccacccggtcggccgggacgtcgagcaggccggtggccagcaggagcatgtccggcgcgggcttggggttcgcgacctcgtcggagccgatgatatggtcgaacagccccgccatgccgagggtggtcagcagcgaccgggcgcgcggcccgctcttgccggtgaccacggcggtgccgaagccgtgctgccgcaggtccgccagcagctccggcgcgccctcgaacacctccacctcacccgccagccggtagctctcgcggacgaacggaccctccatctccagcggcaggtccatgatccgcatgatgtccgggaagtaccgccccaggtgccggttgtactcctcgaacggcgcgggcccgtcgccgacgacctcggcgtaggcgatctcgaacgcctgccgcatgacggcgaagctgttgaccagcaccccgtcgaggtcgaacagcacggcccggtcgtaggtcgcgccggggacgtgccggtggggcgcgggggtcggcggggcgagggggcgcggggccgcgggcgccggaaccgcggtcgcggcccgctcgtccccggctcgggcccgcacgactcgggggttggtctgtccggtggtcatcacggggctcccgtcgggacgaggtcgaccggcgcgtgtcgtcgttcggcgcaccgcacggtgtcggcggcgcggtagacccgttcgatcgcgcccgcgatccagcgcgccccggacgccgcctcgccgcgcgtggcggggtcggccaggcgggtgggcaggctcgcgagctgggcgtcgtactcggcgcccaccggctcggcgggcagctcgaccggggtggtgcggccgtcggtggtgagcaggagccgcgacgggccctcgcggttcgggctgaagccgaaggtgcagcgcagctccgccgtgccgccgctgccctccacgcgcacgaccgtggtgtccagcgcctggtgcgaggcccaggccgcgcgcaccccgatcgagatccccgaccgggtgacgaggaaccccctggcggtgtcctccacgtcgccgaccaccgggtccaccggcgccccgtcgccgcgccaggcggcccggaacgcgtggtcgttgacgaagtccgcggacaccgcgccggtgacgtgctccagctccgcgccctccgggtcgccggtggcgccgcgcagcagcacgcgggcggtgtcgagcaggtgccagccgaggtcgaccagcgcgccgccgccggagcgggtgcggttggtgaaccagccgccccggtccgggatgcccttggaccgcacccaggacacgtcgacgtgccgcagcgcgcccagcgacgcggccacctggcgcagcgcccgcacgtcggcccggtgccgggcggcgctcccgcccagcagcaccgcgccaccggcctgctcggcggcggtgagcgcggcggcctcggcggaccccaggcacagcggcttctccaggaacaccgggacgccccgccgcagcagaccggacgcgaccggcgcgtgcaggtggttcggcacggcgaccacggccaggtcgacctcgtcgcggcgcaggtcctccacccgctccagcgcggtgatcccgcgggagccgggcacggcggcgcgggcctgcgcggacggctcgaccacggcgacgacccggaaggcggggctgcccagcaaccggggcagccacacctcccgcgccacccacccgagcccgaccaccgcgacccgcaccggaccaccgctcccggccctcggcccgtcgctcacaccaccacccccgctccccgcgcccgccaccccgcgctcacgcgcccgcgaccacgtcggccacgacggcggcaagccggtgcagctgctcctcggtgcccagcagcacccggtggtgcagccacacgcagtcgcgggtgatctcctccgacaccgggcaccgggcggccagctcctcggtggtcaggtcgggcgcgccggtctcccagaacgcctgggtgcggtagaccgcccggaacgccatgaacgccgggatcccgcgccgcaccagctcgtccaccaccgcgttgcgccgctcctcggtgacgccgggcatccggaacatcgccatgtagctcgggttgcggtcgctgcgcgggtcgacggtctgcggcacgacgccgtcgatccccgccagcagcgcggacagcaccggccagcgggcctgcctggtcgcgatctgcgagtccagcctgccgagctgggcgcgcagcacggcggcggagaactcgttcatccggaagttcgagcccgaggtgaggtggaagtagccgcggtcgcccttgggcctgccgcagctgtgcaggacgaacgccttctcccactgggcctcgtcctcgaacagcacggccccgccctcgcccgccgtcatcagcttgccgttctggaagctgaacgtggcgatcgacccgagctcgccgacccgcttgccgcgccagtgggcgccgtgggcgtgggcggcgtcctgcaggaccggcacgccggtgctcgtggacagcttgtccagccggtccatgtcggcgaactggcccgccatgtgcacgggcatgatcgccgaggtgcgggaggtgacggcggcctcggcggcggcgacgtccaggcagtaggtgtcggggtcgacgtccacgggcacggcgaccgcgccgaggcgctgcacggcctgcgaggacgagatgaaggtgaaggcgggcacgatcacctcggcgccggggcccacgtcgagcacctggagcgccagctccagcgcgtgcgtcccgttggtgacggcgagcgcgtggcccgcgccgtggtactcggcgaactcgcgctcgaactcgtcgacctcgctgccgccgacccgccaccactggccctggtccagcgcgcgcagcagggccgtgcgctcggcgtcgtcgtgctgcggccaggccgggaactcgatgcctgcgtccggagaattgctcatgagcccctgtcccgtcgttcgcggaaatggcgcgggggaattcgccgcggcctgctttcggaattcgacgctaccgattccgcagatcccgaccaacccccttgacctccccctaatcccccctgttcccaggccatcaccgcagcacgcgggcacagcggcacagccgtgcgcacaatgggggcgaacgggaaccggggcgtccgcgcgccccggcggcgctttcggggaaaggtgtcaggcgtgggcgagctgctgctggtgaacgggccgaacctcggcatcctggggcgccgcgaggtgtcggtgtacgggaccgacacgctcgcggacgtcgagaaggcggtcggcgaggaggtcgccgggcgcggctggtcggtccgctcggtgcagcgcaacggcgagggccagctcgtggacgagatcgaggcgtcctacgacacggtgggcgcgatcgtgaaccccggcgcgctgatgatggcgggctggagcctgcgggacgcgctggcgaactacccgcgcccgtggatcgaggtgcacctgtcgaacgtgtgggcgcgcgagagcttccggcacgagtcggtgctggcgccgctggcgagcggtctcatcgcgggcctgggcgcgcgcggctaccggttggccgcccgcgcgctgctggacctggtggactgaccgccgtcgcgcgcgagcccggccgcgtgcacggccccgcgcagcgaggacaggccgccgagcagcgcgggccgcacgggcgcggtcgggtggccgggccgggcgagtgcggcgcagcggtcggcgaccgcgtcgacgtatccgggcacggcggcggcgaacccgcccccgaccacgcacagcgagggccgcgccagctcgccgacgccgacgagcgcggcggccagcgcccgggccccccgctcgaccgcggcccgcgcccacccgcgcccgtcgccgagcgcccgcaccaggtcctccccggtgaccggcgcgccgccgagcctggcggcctcggcgagcacggccggtccggacgcgaacgcctgcacgcacccggcccgcccgcacgggcagggcggcccgtcgagcgccaccacgacgtgccccagctcgcaggacccgcgctcgggtccggggaacggcaggccaccggacacgacgcccccgccgacgccggtccccacgcccgcgtagaccaggtcggcgcacccgtgggcacgggcctcggcgagcgcggcgaggtcgccgtcgtccgcgacgagcaccggggcggccagcccgccgaggaaccccgcgaggtcgacgccgacccaccccggacggctgggccaggccgtgacgaccccgccgtcgacggtgccggggaacgcgatcccgaccccgtcgagcggcgccccggcccgcccggcgaggtcggcgacggcgcggccgagcaggtcgaggtcggcgcgcggatcaccgtccccaggccaccggaagccctcccgcagcaccagcggcccgcgttcgagccgcagcgccaccttggtcccgccgacgtccaccccgagcagcgccccgctcacaccccgacctcccgccgtccgcacccctcgccgcaccaccacccgcgcgggccgccgcccacgacgccgcccgccacaccgatacccgcgcggtcctcgctcacgacgccgccacaccaccaccgcacgggcctgcggtcacgacgccgccacacctccacccagcgcaccaccacccgcgcgggccgccgctcacgaggccaccgctcacgacgccgccgcccgccaggccgccaccgcctcggcccgccgggcgtcgccgctctccaccagcgcgaaccggatcgcccgcgtggccgcgttcgccgcctgcagcgcctcgtgcgcgccgggctcggggtcgctgcgcccggtcgccacctcggtgatgctgcgcgccgcctccaggcacgccaggcaggccgcgacgtacgcgtccgccccgcctcccgcgccgcccagcttctccagcagccgcgtcacgtcggcgtgcacctcgcgcacggcgtcctccacccggcccgcgccgggtggtgtgccggggatgggggtcaccgctgcctcccggtgcctgacgcggacccggtcagccgagggccgggatgagttcgacgaaccgaccctgccacaaccggcgcagctcgcgcgtcaactcctcggacggcgcgccgccgaccagctccgccagggtcgtcacgccgtccacccggctgagcagcgcgtgcgcctgcgcggtggtggcggtgacgggcccgtggtcgtactccagcgacacctcgtgcaccggctcgcccgccgccgcgctgcccggcgcgggcgcggtgcgcacggccaggcgggtcaccgggcgcagcctgggcaccagcgcgcccaggtcctgctcggggcgggcccgcaccaggaagccctccacgaccaggccgtccaggtcggtggtcaggaagctggtgagcacgtcgtccaggctctgcgcgatcggctcggcgttgttgttgaacgaggtgttgagcagcaccggggtgccggtcagctcgccgaaccgcgccaccagccggtggaagcgctctcccgactcgggcgtgacgacctgcacgcgcgcgctgccgtccacgtgcgtgaccgcgcccagctcggcccgcctggcgggcagcaccggcaccacgaacgacatgaactcgtggtgccccagcgccccggacaggtcgaaccagtcccgcgcggcctcggcggtgaccacgggcgcgaacggccggaagctctcccgcttcttcaccatggcgttgatccgcgtctggttctccgcgggccgggcgtcggcgatgatgctgcggtgcccgagcgcgcgcggcccgaactcggagcggccgtgcgcccagcccagcacctcgccgtcggcgagcagcttcgccgcggtctccaccgggtccaccagcggcgtcacctccaccaccggcgaccagtccgcgagccgcgcggcgacctgctcgtccgtgccgaggtccggcccgagcgccgccgacaccagccgcgccgacggccgctccagcacgcccagcgcggcggctgcggcgtacgcggcgccctcgccggcgcccgcgtcgtgcgaggcggggtggatgaacacctcgtcgaacagcccggccttgaggatgcggccgttgagcgtggagttgtgggcgacgccgccgccgaacgcgagcgtgcgcagcccggtgacctcggcccagtgcccgagcacgtgcagcgcgatcttctcggtcgcctcctgcagcgcggcggcgaagtcccggtgcgcctggctgaacggctcgtccttgcggcgcggccggaacccggcggcgaccagcgcgggggtgaccaggttcggcaccttggtgttgccgatcaggtcgtactcgcccttgtcgcgcagcgcgtgcagcccggagaagacctcgcggtaggtcgacgggtcgccggacggggcgagccccatgaccttgtactcgtcgccgaagccgtagccgagcaggaacgtggcgttcaggtacagcccgccgagggacttctccaccgggtagtcgtgcagcttctccaggtgcgcgccacgcgcgtggtagaccgtgccggagttgtcctcgccccggccgtcgaagatcaccaccagcgcctcgtccgcgcccgagtgcaggtaggacgagtaggcgtgcgcctcgtggtgcggaacgtagacgagcttgtcgtcgggcagctcccagccgaggtcctcgcgcagccgctgcttgatcagctcgcgggagaaccgcagcggcaccctcgggtgctcggtgtagacgtggttgagcaccaggtcgaggtggtcctcggggaagtagtagccgaccgcgtcgacgtcgtccacggtcgcgcccgcgagcgccaggcactcgcggatggcggtggacgggaacttggtcgtcttcttgacgcggttgagccgttcctcctccacggcggccacgagctcgccgtcgcgcaccagggacgctgccgcgtcgtggaagaacagctccgacatcgacgggacgaggtcggtctcggcgggcgagaagttcccgttgatcccgagcacgagcatgtggcatcaccttgatccgggaggcgagggctggggcgcggagggggtcgccgtcaggcgcggggcgcgacggcggcgaggtcgggcgaggtcagccgcagcgcggtgggcgcgggcttcgcggtcggcacgaggtggaagcgctccacgccctcctcggtgggcgcgggcagctcggcggcgcaggcgcagtcggcgggggcgaacccggcgaaccggtaggcgatctccatcatccggttgcggtcggtgcgccggaagtcggcgaccaggtgcgcgcccgcgcgcgccgcctggtcggtgagccaggtcagcagcgtcgacccggcgccgagcgagacgacgcggcacgaggtggccagcagcttgaggtgccacacccgccggcgccgctccagcagcacgatgccgaccgcgccgtgcggcccgaaccggtcggacatggccacgaccagcacctcgtgcgcggggtcggcgagcaggccgcgcagcgcccggtcgtcgtaatgcacgccggtggcgttcatctggcttgtgcgcagggtcagctcctcgacgcgggtcaggtccgcctcgcccgcgcgcgcgacgaccacctccaggtccagggtgcgcaggaactcctcgtcgggcccggtgaagccctcgcggctggcgtcgcgctcgaagcctgcgcggtacatcagccgccgctgccgcgagtcggcggtgaccacggcggggctgaactcggggcgctcgggcagggaggcgacgtcggcctcggtgtacaggcgcacctcgggcagggcgcgggccacctcggcgcgctcgacggggctgtcgtcgacgaacgcgatcgtgcggtgggcgaagccgagccggtcggcgatggcgcgcaccgacgccgacttggcgccccagccgatctgcggcagcacgaagtagtcggcaaggcccaggcgttcgagcacgggccaggcgtggtcgtggtcgttgcggctggccacggactgcaggacgccgcgcccgtcgagcgcggtgatcacctcgcggacccgctcgaacgggacgacgtcggcgtcctccaggagggtgccgcgccacagggtgttgtccaggtcccagaccaggcacttgaccgtcggtgcgggggtctcggtcacggctgctgctccctgagcggagttcggctgcgctggtcaagtccgcgcggggcccggctgcgcacgtgctgggcgagcacgagctggcagatctcggaggtgccctcgatgatctccatgagcttcgcgtcccggtgcgcccgcgccaccacgtgcccgtcgctggcgcccgcggagccgagcagctgcaccgcgcgcccggacccggcggcggcctcgcgcgaggccaggtacttggcctgcaccgccgccaccgccaggtccggcgagttcgcgtcccacagcgcgctggcgtgctcgctcgcgcgggcggcgacctgctcgccgacgtgcaactcggccaggtgccgggcgacgagctggtggtcggccagcacgccgccgccctgctcgcgggtcgtggtgtgctcgacggcggcggccaggcaggcgcgcaggatgccgacgcagccccacgccaccgacacccgcccgtaggtcagcgcggcggtgaccaccagcggcagcggcagcccggtgccgcccaggacgtcggcggcgggcacccgcaccccgtccagggtgatcccggagtgcccggcggcccggcacccgctggggttcggcaccctctccacccgcacgccgggcgcgtcggcgggcacgacgaccgcgctcgccccgccccggtagtgcccgaagaccaccagcaggtccgcgtagtgggcggcggtgatccacgacttgcggccggtcaccaccacctcgccgtcgccggtgtcggtgatggtcgtggtcatcgcggacaggtcgctgcccgcgcccggctcgctgaacccgaccgccgccagcccgcccgaggtgagcctgcgcaggaaccgctcgcgctgctcggcggtcccgagcctgcgcgcggtccacgccgccatgccctgggaggtcatgacgctgcgcagcgagccgcacagctcccccaccgacgcggtcagctcgccgttctcccggctgcccagcccgaggccgccgtgcggggcgccgacctgggcgcacagcacccccagcccgcccagctccaccagcagctcgcgcggcagctcgcccgccaggtcccacccggcggcgcggtcgccgacgcgctcggcgaccagcccggccagtgcgacggcgtcgctcaccgccccgcctcccgcagccgcagcaccagcgtggtcatggtgttgacggtgcggaagctgtccaggcccaggtccggcccgtcgatcacgacgtcgaaggtcgactccaggtgcaccacgagctccatcgcgaacatcgaggtgacggtgccggacgcgaacaggtcggtgtccggctcccaggtctgcttggtgcgctcggcgaggaacgcctgcacccgctcggccaccgcgtcggcggtgagcgcgccgggctgggaggaggtcgtcacagctgtgccttcccgtagtcgtagaagccccgcccggacttgcgcccgaggtgcccgtcgcggaccttgcgcagcagcagctcgcagggcgcggagcgggggtcgccggtgcgctcggccagcacgcgcagcgagtcggccaggttgtccaggccgatcaggtcggccgtgagcagcggtcccgtgcggtggccgaggcagtcctgcatgagggcgtccacggcctcgacggaggccgtgccctcctggacgacgcggatcgcgtcgttgatcatcgggtggacgatccggctggtcacgaagccggggccgtcgccgacgacgaccggtgtgcgggccagctcgcccagcacgcccacgagggtctccagcgcgtccgcgccggtgcgcgcgccccggacgacctcgaccgtggggatcaggtacggcgggttcatgaagtgcgtgccgatcagccgcgccgggtcggggacgtgcccggccagctcgtcgatcgggatcgaggaggtgttggacaccagcggcacgcgcggcccggtgagcgcggcggccccggccagcacctcggccttgaccggcagctcctcggtgaccgcctccaccaccagcgagacgtccgcgacgtcggcgagcgaggtggtggtgagcagctcgccccgctcgcggtcctcgggcagcgcccgcatcagcctggccatgcgcagctgggcggccaccgcctcccgcgcccgcccgaccttggcccggtcggtctcgaccagcaccaccggcacgccgtgcccgacggccagggaggtgatccccaggcccatcgtgcccgcgccgagaacggcgagcaccgtcctgccgtcctgctctcccatcgcgctcccccgccgcggccaccgcggccgccgtccggtccgcgcgccgtcccggcacgcgcattccaccctcgatcgtgtgccgggaaaggcgcgcccgaccccctgacctgcccccctgaacccccctcaacggaaccggaaatcgaatgtcccgaacgcgccgtcaaatcgtcgattgacagccgcagaactgttcatagactgtggcggcagtaccgatctccgaattccacggaagagtcctcccccatggctcagcagatcagcgccacctcggaaatcctcgactacgtccgcgcgacctcgttgcgcgacgacgacgtgctcgccggtctgcgggagcggaccgcggttctcccggccgcgtccgcgctgcaggtggccccggaggaggggcagctgctcggcctgctggtgcgcctggtcggcgcgcgctcggtgctggaggtcggcacctacaccgggtacagcacgctgtgcatggcccgcgccctcccgcccggcggacgtgtcgtgacctgcgacgtcgtcgcgaagtggccggacatgggcaggccgttctgggagcgggcgggcgtcgcggaccgcatcgacgtccgcgtcggcgacgcccgcgccaccctggccggcctgcacgccgagcacgccgtgttcgacctggtgttcatcgacgcgaacaagtcggattacgtccactactacgagcgcgcgctgacgctgctgcgcaccggcggcctggtcgtcgtggacaacacgctctttttcgggcgggtcgccgatccgtccgcgaccgatccggacaccaccgccgtgcgcgagctgaacgcgctgctgcacgccgacgagcgggtcgacatgtgcctgctgccgatcgcggacggaatcacgctcgccgtgaagcggtgaacccgcccgaatcgcgccgaattcccccggagagaaaggccgccgcagtgttcaccgaggacgtggccaccgacctgcccgcctacccgttcctgcgggaccggggcgactgcccgttcgcgccacccccgcgctacggccaattacgggaggagcagcccgtcaccagggtccgcctgtgggacggcagcaccccgttcctgctcaccggtcacgaggtgtgccgcaccgccctgaccgacccgcgcttcagctccgacggcgccaaccgcgcccagccgcgcttcgtgaagttcgacatcccggacgacgtgttcaacttcggcaagatggacgacccggagcacgcgaggctgcgccgcatggtcgccgggcacttcgcgagccgccccgtggaggcgatgcgccccgcgatcaccacgatctgccacgcccagctgcgccagctcgtgcaggcgggctcccccgccgacctggtggcccactacgcgttcccgatcccgtccctggtgatcggcggcgtgctcggcgtggcgggccccggcctggacgagttcgcgcgcgactcgacgcgcgccctggacccgtccctgtccgccgaggagatgggcgccgccatcaactcgatggtcgggttcgtggacgacctgtgcgcggccaagcgggccgcccccggcgacgacctgatcagccgcctggtgctggacttcgagcgcaccggcgagctgacccggaagcagctcgtcgccaccgtgatggtcgtgctgctggcgggctacgagaccaccgcgaacatgatcgcgctgggcacgaccgcgctcctgcgcgaccccgagcagctggccttcctgcgcgccgagcccgccggtttcgccaacgccgtcgaggagctgctgcgctggcacaccatcgtccaggacggcaccggccgcgtggccctggacgacgtcgagctggacggcgtgctcgttcccgcgggctccggcgtgatcgtcaacctgcccgcggccaaccgcgaccccgacgtcttccccgatcccgaccgcctcgacgtgaccaggcacaacgcccggcggcacttcgcgttcggctacggcgtccaccagtgcgtgggcatgacgctggcgcgcgtcgagctgcagatcgcgctggagaccctgctgtgcggcctgccgggcctggcgcctgccacgccgttcgaggacctggacttcgccctggagtccatgaacctcggcctgcgctcgctgccggtcacgtggtgagcaccgaccgtccaccaggggagagccgatgacccgcaccacccccacccccgacctggccccggagttcccgatgcccaggtcgcccgagcacccgttcgacccgccccctcgactccgcgaggcgcaggaggcgggcggcctgtcgcgggtgcgcctgtgggacggcagcaccccgtggctgatcaccaagcacgcccaccagcgcgagctgctgcgcgacccccgcctcagcgcggacttcctgcgccctggctaccccagcccgattcgcatcgaggacaagtcgacgttcatcagcagcttcccgctcatggacgaccccgagcacaaccggcagcgccggatggtcctgggcccgttcaccgtccgcaaggtggaacgcctgcgcccgttcgtgcagcggatcgtcgacgagaagatcgacgaactcctcgcgggccccaacccggtcgacctggtcaccgcgttcgcgctgcccatcccgtccctcgcgatcagcgccgtcctgggcctgccctactccgaccacgaggtcttcgagcgcaacagcgccgtgctgatccgccaggacgtgcccccgcaggaacgggccgaggccagcgaggagctccagcaccacctcgaccgcgtcctgggcgacaagatgaccgaccccgccgacgacctcctctccgacctgggcgcacgggtgctggcaggcgagatcagcaggccggaggcggtcgacatgaccgtcctggtgctggcgggcgggcacgagaccaccgcgaacatgatcgcgctcggcaccctcgcgctgctccggcaccccgaccagctggcgctgctccaggcgggcgacgaccccgccctcgccgagaccgccgtcgaggagctgatgcgctacctgacgatctcgcacaccgggatgcgccgcgtggcgaccgaggacgtggagatcgacggccaggtgatccgcgcgggcgagggcgtggtgctggcgacctcgatcggcaaccgcgaccccgacgtctacgacggcgacccgcacgtgctggacctgcgcaggccggtgaagcagcacttcgcgttcagcttcggcacccaccagtgcctgggccagtcgctggcccgcatggagctgcaggtcgtcgtgaacaccctctaccgccgcgtcccgaccttgcgactggcgaccgcgctggagcgcatcccgttcaagcacgacgggatcgtctacggcgtctacgagctgcccgtcacctggtgaccccgtcccaccagacctcctgccacgcagacctcccgcaagccgaccccgaaaggccgttcccatgagcgacaccacgctgtccgtgcccgtccccgaggaggtcggcaagctctacgaccagatcctgaaggacgagcacacctacgagcagttcgagaagttcaaccaccagctgcacatcggctactgggacgacccgacctcggacgtgcccatgcgcgaggccgtggtgcgcctgaccgagctgatggtcgagcgcctgcgggtggacgccgaggaccgcgtgctggacctgggctgcggcatcggcggcccggcgacccagatcgtgcgcaccaccggcgcacgcgtcgtcggcgtgagcatcagcgaggagcaggtcaagctcgccaccaggctggccaccgaggcgggcgtgggcgaccgcgccaccttccagcgcgccgacgccatgcggctgccgttcgaggacgagtccttcgacgcggtgatggccctggagtcgatcctgcacatgccgtccagggagcaggtcctgtccgaggcgcgccgggtcctgcgccccggaggccgcctggtcctcaccgacttcttcgaacgcgcaccccgcacgccggggatgcaccccgcgatcgagggcttctgccgaaccgcgatgacgacgatggccgacgtggacgactacgtgccgatgctgcaccgggtgggcctgcgcgtgcgggagctgctggacatcaccgagcagaccatggaacgcacttggcgggagaccctggagatcgtcagccagaacgaccgcccggtcgacttcgacctggcggagctgttcggcgtggacgagttcggctgcctgctggtcgccgcagaccgcccgtgaggcccgtccccgaggccgtgggccgcctgtacgacgacctgctggaggccgagctggaggggggcgcagccgacccgaacctgcacatcggctactgggacgcgccggactcgccaacgccacgcgcggaggcggtagtgcgcttcaccgacgaacacgtccgccgcctgcacgtgaccacgggcgaccgagtgctggacgtgggctgcggcgtaggcggcccagccctgcgcgcggtggacctgaccggcgcccacgtgaccggaatcagcatcagcgccgcccagatcacccacgcgacccacctggccaagtccgcgggccacgcggacaacaccaagttcctccacgcagacgcgatggccctcccgttcccggactcctcgttcgacgcggtcatggcgatcgagtccctgatccacatgcccgaccgcgagcgggtcctgaacgaggcaagacgcgtactgcgcccaggcgggcgactggtcctcaccgaactgttcgaacgcgccccaagacccacccgcagacacccagcgataaccgagttctgccgagcatcgatggtgtccctgcccaacgcagacgactaccccgcactactacaccgagcaggcctacgcctacgggaactcctggacatcaccgaccacaccgtccaacgcaacttccgcgaactggccgatctggtaggcgacgcgaagggcctgctgttccacccacgcgacctggtgggcgtcccagaattcggctgcttcctagcagtagccgaacacccgtaaccacgcggtggcgtcccccacggacgccaccgcctcgcgggctgcggggcgagcgcagcgagcccgcgcagccccactcccgcgtccctcttctccgtgtggcctggcgcatgtcaaattcccactgactgccaacagatcatgtgccgtttgagcaggtcagcgacttgtcgcgcttcggtgccttaaggccgagctgggatgggggcactgtttccggactgagcggggcagcttggaaggtggagttcggtgagcagaggcagcacgtcccgtcgcacgtagaggtggttgtacacgcggtggcgggacctgcgcagtaggccgctatccgcaagctgctccaagatcaggagtgcggcgcggtgcgtatagccgagttcggcggtcagcatggtgctgttgagcagtggggcgacgagcagcggggcgggaagcgctttgaccttcctccgcccggtgcgcatcgcccaggtgggcgatcgcgcgagcctcacggatcgcggtcacctcatgcaggctggcgctcaacctggaacgcgcgactgtttcgtccagacgtgccagggcggtgtaggcgtgcaacaaggtcttgctggtttcggagcgcagtctgagccgggaccaggacgacaactccgcgatcctcgcggacgggggcggcctcgtgtcttcaccggtggtagttgacctgcgcggggcggaggtgccctattgctgccgggacgaggtcatcccccggagcagtttctcagcacgccgtgaatcgagatccggggcgctgagcgcggtgaacgcctcgtccagcgagtcgcacgcgcacgtcgtcctgacatcgggccgcgcatggcccgaggtggtcagcggtgagcgggaaggcgcggcagggtgtgtgcgagacactccgggactccgtgcagaaggtcgatcaggcgaaagggttgaactgcgaatcgcaaagcggcccggccgcaaaggggtcgggccgcctgcgacgattggtcacgctgctgcggcgcggtcccgccggaactgcttgccgagcaggtcgatccgccccttgtgatcttctgccagcgcctccagaaccgagagcagtcgtcgggcgtgcagtgcatggccaataccatcgtcgcgtaccccagagggtgtcgctcccgttcaggggcgaccatttcccacgcccgcttggcctccttggcggcccggccaagatcgccgagcatcaggtaggtgcccgacaacccgacaaccctgcctgccaacgcggcttccggcaccccgcgcgcctcgtcggcttccaacgcccgaacaccgtgccacagcacggcccgcgcgttgccctcgctcgtctccagccatcccatgacaccgtgcgcttcggccagtgaccacgatcggctgtcgggatcggtgttgcacaacgccagctccagcgctcgttcagcagcgttaccgaccacagcgcggcgccgatgtccagcacttcttgccggtacccgcccacgagtgcggcggtgcgctgcacggccacgacttcccgccgatgcacgatcagccacttgtacgccgccaaggcgttgtcgaacagcggcttcgccccgacctcgaagccgtcgacgaacacctcgcgcaaatcaccgagcagtttccctgcggccaaggtgcgccgatgcaggtacctgcccaagcgctccaactcttgcgggaactcctgctgcacagcccatcgcagcagtgcctgggcttggtctgtctcctgcgcgcgatggcgaccggccagccggtaacgcgaggaggtgaactcggggtgcgtgatgttcgggtgaagttcagtccacgaaggctgcgtcagcaccagcacgccctggttcacccagtcccgcgcgtgttgggcggtctcctcgacggtgattcccagcatcgcggccaacgacgaggtcgagaccacggggtccggcagcaggtccagcaatctcagctcttgcggaatgggcacaagagtgttgatcatcgatgcccctcccggaggacggcgatgattggagtggcgaacagaaggggaaacgccagttcgccgggttccggcggtccacgcgccttcggccggccacttggactccgacgggcagaagttcaccggcaaggactctggtgacggtggagcggtgcacgcccatcgcttcggccaaggcgtagtcgctgtggtaaccagcgaactgagctagctttcgcatcttgtcgccgcgcaccccgacgaccttcttgatcttttcggtctcgctgtcgttgtcgtcgacatgtccgccgtccggcgctgacaccgttctccttgagatcgccgagctgaatgggggatgcttcgacgtaaggcgttgcgtatgcgcaacaggtcaggcggcgtcgagtctccccattaccgaggtttcgcttgatcgccgacggggcccgcctcgaagaagtccaatcgagctggcatccccttcgattgatcaatagcgcgacgggtgtcgctcgacatcgccccaccgcctgctcctgacgtgccacgagcagggaggagcgacctccctcgggactgcaccgaccgttcctccctgtccgccgattcagttgcattccgccacgctaggtgccggatgcgggccgaagggacaacgaagggacaagtcgaacagcccaggtgcgaggtatcttgaaatagcccgaatcctccgtcgcgaagcaggtcgccatgcccactgacgaacagtccgagggtgtcggagagcgcatagccgtccaacgcaaactggctggcttgactcagcaagctctggcgaagcgcgcacacgtcagcctcagcctcatcaaaggggtggaacagggaaggattcccgcctctcccgcgctcgtgtcccaggtctcgcgggcgctcaaggtcgaggcgacgatcttgctggggcagccgtaccgccccgaggatcggagcagtcttcgcgttcactccgtcatccccggtctgcgccgagccttggcggcctaccggttgcccgctgatgagggcatcagccctcgcgggtacgacgagctggccgccggtgtagccgccgcgtcgaagatgcgccacgccgcgacgttggacgtcctgggggctgaactccccggcctgctcgacgagatccgctcggccatcgacgaggctcggggagttgagcggcagcgcctgttcagcttgctggcagaggcatacgcagccgctggtcaagtcgcgtggaagctgggttacgcggacctgtcctccctggcgacggagcgcgtggagtgggcggccaaagagtccggcgatccgctcgcgatgggcgcagcggacttctacatcgccggtgagctgatcgcagcagcggagtggcgcggcgccctctcctacctcgacggctcccgtcgccgcctggagcacgtggtgcgcaaggacgacgaggccgccttgtcgatctacggagtcctgcacctgaagtcggggctcgcggcggcacgggccgggaaagccgacgaatccgacgcgcacctcgctgaagcccgtggcatcgcggaaagggtgccgctgggcagtgaccactaccggctcgcgttcgaccgggactcggtcaacatctggaccgtggggctggcagtggagcgcatggacggcacggaagccgtcaaacgagcccacgggatgcgcttcagcaagaccaccccgcgtgaacgcgtgggccaccactacatcgatctggcgcgcggctaccagctgcacggagaccgtgaccgcgccctgcacacccttcagatcgccaggcgaacctcaccgcagcaggtgcgctaccacccgcaggtcagggaaacccttctcacgctcgcggaacaggaccgcaggcgctcggattccctggcagggctcgcgcgctggatcggtatgccggtgtgacaggacggcgagctgacgtcgctgttgaggggcagccccccatcggccgcccctcaacagcaggtgccggtacgtccctcacagcgcgacgctgacgatcaggctggcgaacatggccacggccagcgcgatcatctgcttgggcgcgccgccgaagaagagggaccccacccaccgcagtggtaaaccggcaccgagcaccaacggccaccggccggcagagcccgtccccctcttttttggaggtctgccccgccggcgaggcgtgcccttcagctctcaagctctccactctcgatcttgtggtccgaacaccccccgcacccccactacgatccccccatgggggctctgatcatcgcggtagtgctgctgctcgtcttcctcgtgcaactcaagcgggaacccagacgactgggcaacggcgtctacctgctgatgagcctggcgttcttcgccctctggctgctcaccctcgccaccccccagaccaggacgctggtggtaggcgcggtagtcctgatcgccccggtattcgtcaccgtgatcgccctgttcctcatcgccaacggcgtcaccctgctgcgccgcgagggcgtcaaaccaggcaacgccctctccttcggcgcaggcaccgccatcctgtgcgtcgtaggcggcctgctcctggtcctgctctccgccctgcgcgaaggctcccccgacccctgggtgctggcagcagccggttccctggtcctcctggccggctacctgggcttcgccttcaccctcttcctgctctactccgtgctctacggccgagtccgcaagcgcaccggccacaccgcgatcatcgtcctgggcgcgggcgtccccggcggccgagtgaccccgctcctggcaggccgcctggaccgcgccctgaagctctaccgccgcgccgcagccaagggcgcttcccccgtggtagtcgcctctggcggccaaggcccagacgaaccagcctccgaagccgaggtcatggccaactacctccgcgaacgcggcatcccggacgaggccctcctggaagagcgcgagtccacctcgacctgggagaacctccgcctctcctccgccctgctcgccgaacgcggcgtgaccggcagactcctggtcgtcaccagcagctaccacgtcccccgagccgcgatcctctcccgccgcgcaggcctgaaggcagacgtccgcggcggccgaaccgcctggtacttcgtgccgaacgccttcctccgcgagttcgccgccctcctggtccagtaccgcaccctcaacgccctggcagcctgcaccgcactctccgtcttcccgctcctggcctacggcgtctgaaaagcacgacccggccgaccggacaccgcgtcacagatccagcggcgccgacccgaaggccacgttgaaccggtcgcaccacaccacgacgctgcgcagccccgacaggtccacgtcctccggaatcaggtagttctggttgccgtcggtggccttcatgggaccgagcggcaggtagcgcccatcgtcgtacttgccccactccccacccgcggtcgcgtcggagagccagatgtgcaggtcgggcccgtccgaggtggagaacccatccagccgcagcacgcgcgcggccccgctgcgcagcacggtggcggtgccccgcgtctcgtgctcctgggtgacgaacccacccgttgccagcaccgtcggctggtcggcggtcgccgacgacgtcccaccgggcgtcgtggcaccgctgcccgccgccgccccggtgctcgacgccccagccccggtgctcacccccgcaccggtcgagacgctgaactcggcgggcagcgcctcgtccgcctcgctgcgcgtccacaaccgccacggctggaacacccacagcccgacgaccgcagccaccaccacaacccccgacaccgcccaaaccgctctcctgcgcaccgaaccgcgcaccacgtcccctcccgttctccgcagacgacctgccaccatgccacgggtcgcgcccgatgaccacgaccaccgcgccacacccgccccacgcagcgactaggctgcccaccggggtcgccagccgatcccgagcgggttgagcaggcagcccaccgcagttcgcgctagtgggatggagggagcgggccggtgtccgagctggatgcagccgcggtggtcacggtgggttccgacgtggtgcgcggggtgcccgtgctgcgcgtcgccggggagatcgacaccaacgtcgccgacgaggtccgccgggcgctgctgccctggctggacgggttgcgcgggccaggggtgctcgacctgaccggggtgaggttcatggcctccaccgggttgtcgctgctgatcgaggccgcccggcgcaggccggcgaagctggtgctggccaccgcccagcgcggcgtgctccggccgctgcagctgaccgggatgagcgcgctgctcccgacgcaccccaccgtggacctggccgtggacgcccagctcggggccgccctggccgggatgcccagcacggcctgaccaccctcggtccacgggcggcctgcccgcggaccacccgcacggcgccctggggggacgagatcacagctggtggaagacgcgatcctggtccgcgcgccgcgacgccggtgggcgcgggcgctcccgccacggcggcggacccgcccccggtccccaccacggctccggcaccggccccgacaccgacaccgaccccagccccgcccctgggcacgaccacaccaccaaccccggtcctgggcgcaggtgtcgccaccgccaccgccctgacgctggcactcgccggggccgcagccccagccgacaacagcgcgggaaaggcggccatcatggacgaggtggacgccccaaccaccccacccaccccggccgcgctggacctcacgccccgcccacccctggccgaggtgcgccgctggaccggcgcgctgctgatcgacgccgacgaggaagcagcggacgacgtgctgctcgtggtcaacgagctggtcgccaacgcctacgaccacaccacctccccactcgccctgcgcctcaccaccacccccgagcacgtgcgcgtggaggtcgaggacggctcccccgacccaccacgcccggacctcaccgcgggcctgcgccagatcggcacgcgcggacgcggcctgctgctgatccgccagctgaccgatcgctggggcagcacgccccaccccggcggcaagaccgtgtgggcggagctgccgaacgtcccggcgacctgagcccgacgccccaccaacgaggccacggcggatctcacgggaagagcgcggcggggcactccgggcgcgttggacgccggcgcactccccggtgaggggtcgggcggcggagtggatgagcgtggcggcgagcagggccggtccggcggacgagacggccatcagcagcccgccgaccggggccgcgaggcgtcgggcgcgggcgccgagcctgcccggcaccgggccgaccacggagctgacgccgaggacggcggtgcaggcgccgagcgcgcgcctgcgggccgcgccctcgaagcgcacctggacggcggtcagcgccccggagaccatgagcgccgcgcccgcgccctggaccacccgcgcggccaccagcgccgggccggtgggggtgaggccgcaggccggggaggcggcggtgaaggtggcgggcccgacgaggtgggcccagcggcggccccggatctcgccgaggtgggccccggtgatcagcaggacggcgagctgcggcaggacaccgacacgggcacgaccgcgtcgatgtgcgtcgcggcggcgcagggcgcggagacgggcgcgggcgagcgctgagggcccgcccggcgccactcccccgtcacacctcccgccgcagcacatcctcctccgtctcccgccgcaccagcacccgcgccactccgtcgcgcacgcccaccacaggcggcctgcccacggcgttgtagttcgacgccagcgcgtggtggtaggcgcccgtcaccggcaccgccagcaggtcccccgcgcgcacgtccgcgggcagcggcacgtcctcggcgagcacgtcacccgcctcgcagtgcctgcccaccaccgtcaccggcgcgcgccgcccgccccggccgaccaggcgcaccgcgtaccggctcccgtacagcgcgggcctggggttgtcgctcatgcccccgtccacggccacgaacacccgcctcaccccgcgcttgacggcagccacccggtacagcgtcacaccagcgcccgcgacgaccgaccgccccggctcgatcagcagcctcggcaccggcacgcgccgcagcgcgcactcgtggctcagcgccacccgcacccggtgcgcgaacccgccaaggtcgaactccccctcccccggcaggtagggcaccgcgaacccgccgccgaggtccagctgctcgatccgcaccccgcacgaggcgatcagcccgaccatccgccgcgccgcctcctcgtacaccgcgacgtgtcgcacctgcgacccgacgtggcagtgcagccccaccagcctcagcgacggctgctcgaccacccgcagcaccgcctccagcgcgtccccacccgccagggagaagccgaacttctggtcctccaccccggtcgccaccgcccggtgggtgcgcgggtcgacgccgggggtgacccggaccagcacgtcctgcggccccctggccagcgcgcccagctgctcgatctcgtcgaacgagtccaccaccacccgcccgaccccgtacccgagggcggccttgaggtcctcgggcgtcttgacgttgccgtgcagcagaatccgctccgccgggaacccgaccgaccgcgcgatcgccagctcccccgccgagcacacgtccagcgacagcccctcgtccgccacccaccggtacacctcgcggcacggcagcgccttgcccgcgaacaccacctcagcctccggcagcacctcccggaacccgcgcgcccgcgcccggaccgtgccctcgtcgagcacctggcagggcgtgccgaaccgggcggcgagctcggtcgcgggcaccccgccgagcagcagctccccccgctccagccgggtccccaggggccacagccccgcctccagggccggttcgccggtcatgccgacgctgggcagcaactccgcgagtgtcatgcccgccagcacacgcccgaaccggccggggcgacagcggcgcgaacgcgtccctgacggcgtgccgggcgggattgacgccgccctgacccgaccgccccagcccgctctcgaacccggcggaagcacccccgaaacgcgccggaaacccgcccgcgcattcccccgaacgcctacctcacggcgattttgatgctttttttacgccgggacgccgcgatattcactcctccgagccgcgcggggacgttgacttctcatgcccgacgacgtgatcgaggagagaccccgaatgtccgaaacaccggttttcgccgttccacccagggtggaaagcccggtacgcccggccgcgcccgccaaccgggtggggcgctggctgctggagcaccgggtgcaaccggcgggacccgcgggcaccgaccagcacagcacgccccaggcgtggtggaaggtcatgtgcctgaccggcgtcgactacttctcgaccctgtcctacctgccgggcatcgcggcgctggcggccggggcggtctcgccgctggcgacgctgctgatcgtcgcgctgaccctgttcgggatgctgccgatgtaccgccgggtggcgcacgagtcgccgcacgggcagggctcggtggcgatgctggaggacctgctgccgttctggcgcggcaagctgttcgtgctggtgctgctgggtttcgtggccacctcgtggatcatcacgatcaccctgtcggcggccgacgcgtcggtgcacgcgctggagaacccgcacgcgcccgcgttcctgcacgggcacgaggtgctggtcaccgtggtgctgctgctcgtgctgggcggggtgttcctgctgggcttcaccgaggcggtcagcgtggccatcccgctggtcgcggtgttcctgctgctcaacgcggtggtcgtggtcgccggcgtgctggaggtgatcgcgaacccggacgtgctggacggctggttcgcggcgctgacctccaccggcggcggcggggtgctgggcgtggtcggcccggccctgctggcgttcccgctgctcgtgctcggcctgtccgggttcgagaccggggtgagcatgatgccgctggtcgaggcgaagggcgccgacgacgccgaacgcctggcgaaccgcgtccgcaacacccgcaagctgctcaccaccgccgcgctgatcatgtcggtgtacctggtggccaccagcttcgtgaccaccctgctcgtgccggtcgagcagttccgccccggcggcgaggccaacgggcgggcgctggcctacctggcgcacgagctgctcggcgagtgggtcggcacggcctacgacatcagcagcgtgctgatcctgtggttcgccggcgcgtccgcgatggccgggctgatcaacatcgtgccgcgctacctgcccgcgtacggcatggccccggactggacgcgcgccgtccgaccggtcgtgctggtctacacggtgatctgcgtcggcatcacggtgatcttccaggccgacgtggacgcccaggccggcgcgtacgcgaccggcatcctggcgatgatggtgtcggcgtcggtggcggtgaccctgtcggtggcgcgcgccgggcggcggggcgcggcctcggcgttcgcggtgctgaccctgatcctggtgtacgcgctggtggagaacgtgatcgagaagccggacggcatcacgatctcgttcgtgttcatcgtcggcatcatcgccgtctcgctggtctcgcggatctcgcgcaccaccgagctgcgcgtggagcacatcgagttcgacgagaccgcgcgcaggctcatcaccgactcgatcgcccacgacggcgcgctgaccgtgatcgcgaaccgcaggcaggccggtgacgtggccgagtacgcggacaaggaggccgagcagcgcggggtgaacccggtgccggggcaggcggacgtgctgttcctggagatcgacgtggtggacccgtcggacttcagcgacgtgctggaggtgcgcggcgtggaggtgggcggccaccgggtgctgcgcgcggacagcccggcggcgccgaacgcgatcgccgcgatactgctggcgctgcgcgactgcaccggggtgcgcccgcactgccacttcgcgtggagcgagggcagcccgctggggcacctgttccgctacctgctggtggggcgcggcgacacggcgccggtggtgcgggagatcatccgggcgcacgagtccgacccggagcgcaggccgggcatccacgtgggggcctgagcgggcacgacggcggggtggtccaggcaggcagcgtggtccaggccagtggggtgctcccggccagcaacgtgctcccggccggtgggggctccagggcgctgcggcggccgatcgcgcgggcgtggtcggcgaaccgctcgcagtgctcgctgagcagggccgcgtcgacggcggcgtcctcaacgccgcgcagcacggccagcacggaccggggcactcaccaaacgcgaagagccacaccaactgggcttcggcgtgggaggcgcggtgcagcggtttgtggtctcgcgctgccgcgcggcgcgggggactgggtcgcgagcagcacctggccgccgtgccgcgcggcgccccgcgccaggtcgcacacggcggccaggtccggcaccggcgcgtcccgccggtcgtggaacacgtcgcgcatcgcgctctccctcggaggatcggatcggaaggccctgatcccaaccgggcgcgcaccccggcgacaagccctcacccgccgaacttgcgctttccttccgccccgacccccgcccgtcacaaacccccgtcaccccgccgtcactttttgtgatgacgatcaggaaacagtagtagcccattcgtgacctgcactgacgcgcagatcaccccacccgtcaacgaaacgtaaaaccgcctggtcaccccgtcaaagacccgtcagcaccccgctcacggcgttttccccgttgcacccttttggcgtcgcggtccccacgaacgggggccgctcggagtcgggaagggagcacgctcatggccgacctggcctacgcgtcgctgctcatcgctgtgttcggactgctcgtcctcggcattcgcggactggggcggctctgatgggcggcacgggagtcgtggccaacgccgtcggtggcgtgctggccctgctgctcatcgggtacctgttcgtcgcgctgatcaggccggagaagttctgatgtcctcgaccacggcgggcctgctccaggtcgccctgctcatcgccgcgctggccgccgcctaccggccgttcggcgactacatggcccgcgtctacaccgacgccaagcacaccaaggtcgagcgcctgctctaccgcgcagcccgcgtcgaccccgactcgcagcagcgctggggcacctacgcgcagggcgtgctcggcttctccctcgtcggcgtggccctgctgtacctgatgcagcgagtgcagccctggctgccgttcgaccacgaccggggcgcggtctcgcccggcatggcgttcaacaccgccgcctcgttcgtggccaacacgaactggcagtcctacgtcccggagaccgtcctcggccacaccgtgcagatggccgggctgaccgtgcagaacttcgtctccggcgcggtcggcatggccgtcgccgtggcgctggtgcgcggcttcacccgcgagggctccgaccggctcggcaacttctgggtcgacctcaccaggggcaccctgcgcgtcctgctgcccgtgtcgttcgtgttcgccatcgtgctggtcgcgaccggcgtcgtgatgagtctgaaggcgggcgtggacgtggacggccagcaggtcgccatcgccccggccgcctcgcaggaggccatcaaggagctcggcaccaacggcggcggcatcttcaacgccaactccgcccacccgttcgagaaccccaacggctggtcgaacctggtcgagatcttcctgatcctgctgatcccggtctcgctcacccgcaccttcggcaccctggtcggcaaccgcaagcagggctacgtgctgctcagcgtcatgggcgtgctgtggaccgcgatgctcgcggtcatctgggcggccgaggcgcacggcctgcgccccctggagggcaaggagctgcggttcggcgtccccggcagcgccctgttcgccaacaccaccaccgccacctccaccggcgcggtcaacgccatgcacgacagcctcaccggcctgggcggcggcgcgacgctgctgaacatgctgttcggcgagatgacgccgggcggcgtcggcaccggcctgtacagcatcctggtgatggcgatcatcgcgatgttcctggccggtctgatggtcgggcgcaccccggagtacctgggcaagaagctgggccgccgcgaggtgacctgcgccgcgctgtccatcctggcgatgcccgcgctggtgctggtcggcgccgggatctcggcggtgctgccgtcgacggccgggtacctgaacaaccccggcgagcacggcctgtccgagatcctctacgcctacgcgtcggcctcgaacaacaacggcagcgcgttcgcgggcatcaccgtgaccagcgactggttccagtcctcgctcggcgtctgcatgttgctcggccggttcgtcccgatcatcgcggtgctgtgcctggccggttcgctcgcccggcagaagcgcgccccgcggaccgcgggcacgctgcccacggacagcccgctgttcgcctcgctgctggtcggcgcgatcgtgctcgtcgccgccctcaccttcgtccccgccctcgccctcggccccatcgcggaggcactgctgtgaccaccaccgacacccgccagcccgcccccgaggacacgggcgcgcggcccccggccaagcccgtcccgtcgggcgtgttcgccccgcgccagctgctcacgtccctgccggacgcgctgcgcaagctccacccccgccaccagctgcgcaaccccgtgatgttcgtggtgtgggcgggctcggtcctggtcacggtcttcgccgtcaccgacccgaacccgttcacgatcgcggtcgcgctgtggctgtggttcaccgccctgttcgccaacctcgccgaggccgtcgccgaggggcgcggcaaggcgcaggccgagtcgctgcgcaggactaagaccgacgcgctggcccgcctgaccgacggccgcaccgtgcccggcaccgagctgaaggtcggcgacctggtcgtggtcgaggccggtgaggtgatccccggcgacggcgacgtggtcgagggcatcgccaccgtcgacgagtcggcgatcaccggcgagtccgcgcccgtggtgcgcgagtccggcggcgaccggtgcgcggtcaccggcggcaccaccgtgctgtcggaccggatcgtcgtgcgcgtcaccagcaagccgggcgagacgttcgtggaccggatgatcgcgctggtcgagggcgcgcagcggcagaagacgccgaacgagatcgcgctgacgatcctgctgtccacgctcacgatcatcttcctgctcgcggtgctcgcgctccagccgttcgcggtgtactccggcggcgagcagtcggtgatcgtgctgaccgcgctgctggtgtgcctgatccccaccacgatcggcgcgctgctgtccgcgatcggcatcgcgggcatggaccgcctggtgcagcgcaacgtgctggccacctcgggccgcgccgtcgaggcggccggtgacgtggacacgctgctgctggacaagaccggcaccatcacctggggcaaccgccgcgccaccgagctgatccccgcgcccggcgtcacgctggacgagctggtggacgccgcccggttgtcgtcgctggccgacggcacccccgagggccgcagcgtggtcgagctgtgcgcgaccgggcacggccgctcccccgagcccaccgacgcggagaagaccggcgagttcgtgccgttcaccgcccagacccggatgagcggcatcgacctggacggccgcagcgtccgcaagggcgccgcgaccgcgttcaccctcaccgactcggtcaagtccacggtggacgagatcagcggcgacggcggcaccccgctggtggtcgccgacggcgagcgggtgctcggcgtgatccggctgtccgacgtggtcaagcccggcatgaaggagcggttcgccgagctgcgcgccatgggcatccgcacggtcatggtcaccggcgacaacccgctgaccgccagggcgatcgcggccgaggcgggggtcgacgactacctcgccgaggccaagcccgaggacaagatggccctgatccgcaaggagcaggagggcggcaagctggtcgcgatgaccggcgacggcaccaacgacgcgccggcgctggcccagtccgacgtgggcgtggccatgaacaccggcacctcggccgccaaggaggccgggaacatggtggacctggactccgaccccaccaagctcatcgagatcgtggagatcggcaagcagctgctgatcacgcggggcgcgctgacgacgttctcggtcgccaacgacctggcgaagtacttcgcgatcctgcccgccatgttcgccgcgatccacccgcagctggacaagctcaacgtcatgggcctggccacgccgcagtcggcgatcctgtcggcggtcatcttcaacgcgctgatcatcgtggtgctgatcccgctggcgctgcgcggcgtgcgctacaagccctccagcgcgagctcgctgctgcggcgcaacctgctggtgtacggcgtcggcggcatcatcacgccgttcgtcggcatctggctcatcgacctgctcgtccgcctcatccccggaatcgggtgaactccgtgaacgcgttcgtgaagcaggccctggccggtctgcgcgtcctgctggtgctgaccgtcatcaccggcgtgctctaccccgccgccgtctggctcgtctcgcgggtgcccggcctgcacgccaacgccgaggccaccggcaccgagctggtcgtggcgccgcgcgagggcgacggctggttccagccgcgcccgtcgatggcgacgctgcccgcgtcgggcgggtccaacaagggcgagcgcaacgccgactacgacgcggtgatcgccgagcgccgcaccgagatcgcccggcgcgagggcgttgcggaggacgccgtgccgcaggacgcggtgaccgcctcggcctccgggctggacccgctgatcagcgccgagtacgcggcgatccaggtgccgcgcgtggcgcgggagcgcggggtgtcggaggacgccgtgcgggcgctggtcgccgaggcgtcggtgggccgctcgctcgggttcgtgggcgagccgggcgtcaacgtcaccgccctcaaccgggccgtcgacgcggcggagtgagaccgaccgggggccgtcctcgcggcggcccccggtcttccccatttctctgatctcgggagcgggcgggaccgtggacaagcgcaagcgcggcgaactgcgcatctacctgggcgcggcgccgggcgtcggcaagaccttcgcgatgctcggcgaggcgcaccgccgccgggggcgcggcgcggacgtcgtcgtcgccctggtcgagacgcacggccgcgagcgcaccgccaccatggtcgacggcctggaggtgctgccccgcaaggaggtccagcaccgggggaccacgatcaccgagatggacgtggacgcggtgctggcccgcgcgcccgagatcgccgtggtggacgagctggcgcacaccaacgcccccggctcccgcaacgccaagcgctggcaggacgtcgaggagctgctggacgccggcatcgacgtgctgtccacgctcaacatccagcacctggagtcgctcaacgacgtggtgcgccgcatcacccgcgtcgagcagcgcgagaccatccccgacgaggtggtgcgccgcgccgagcaggtggagctggtcgacctgaccccggaggcgctgcgccgccgcctggcgcacggcaacgtctacgccgcgcacaagatcgacgccgcgctgggcaactacttccgggtcgggaacctgaccgcgctgcgcgagctggcgctgctgtgggtggccgaccaggtggacgtggcgctccagcggtaccgcaccgagcagcgcatcaccgacacctgggaggcccgcgagcgggtcgtggtcgcggtgaccggcggcgcggagagcgagaccctgatccgcagggcccgccgcatcgccgcgcgcgccggggcggagctgctggtggtgcacaccatgcgcggcgacggcctcgcgggttccgcgccggagtcgatccggacccgcgtcgggctcaggtgctcgacggtgctcttcaacgtggtctcctcgtaacgggacgtgcggaacaccccgcagcgcccagggtcgggcggctgacgggattcgcctgagtctaggcgaggccgcccccggccggggtggcaccccgcgaccgggtggttcacgtgcgggtgcgcgcgcccggcgcggcgcgcgcggtgcgagaggtgggccgtccggcggcgcgcggttttccgacatggcgcgcgcacgaaatagttttcggcgggtcgggcgccgtcgaatcgactcggggtcgggttttccgcgccaccccggaagcggacgaaccgggcgggcgaaccgggcgggcggtgcgcggacaacgggcgcgaccgccgcggtgcgccggtttgggcagcctttaccgccctccaggtcacccattccgccgttgcggggaacatccgcgtaccagtggcccccggcggacacgcggcccagcacccgctaggccgttcgcaggacgtcgtggtgcaccgggagcgtgaaaccgaacgtaaccggacagcggcgggctcaagtggggtaacactggcgccgcagcgcactcttacccacagcgacgaacgcggcggaacgctaccctttacaggtgaagtgaggccattcggagcaccggtgcgcagaaaactttcacgcccggagatgactccactcgccgtagtccattagtgtgggattccggtaccgttgcgccgcaggccgcaagaaggcggccaggaaagacgattaactcatccgggcgccccgccgtcgtgcacgtgaacgcgacgggcgaccgggaacggaacgagcgagacatgtcatcgcgctctttaccacctaccagaaaaggtgccgatgaccccgatgaagaccattccgccgattcccccgaacacgcgggcgtccgcccgtccgccgctcgggcaaccgcacgacgggcttgcgcgccacccggaaccccagggcccggccgcgaggcgatgttgacccgacccccggccaccagccgggacaggccgaccaccgccacgcgcggaacccacggcgaaccgcctctcgccgtgatccaccacggaccgttggggagttccatggagacccgtcaacttctggcgttcaccacagtggtgcagaccggcagcttcacgaaggccgccgccacgctgaactgctctcagcccacgatcaccaccaggatcaaggcgctggaggagaccctcggcgtcgccctgttccgcaggttgccgcgcggcatccagatgacctccgccggggtcgagctgctgccgttcgcgcgcaacatcatcacgctcaccgacaaggcccgcaaggcgatcaccatgaacggggagccgcacgggcacctcgtgataggcagcgcccagagcctcaccgactaccggctcttacccctgatcgagtacatgtgctggcgctacccgagcgtccagatctcgctgcactcgcgaacaacccggtcgaacctggccgccgtgcgcgagggcaggttggactgcgcgttcttcatcggcccggtcgagcagcgggacggtctggagacgacggtgctgtgccccgaaccgctggtgatggtcgcgggccccggccacgcgctggcgcggtcgggcgcggtcaccgaggcggacctgcggggcagcacgctggtcagggccgagaacggggcgagctaccacgagcagttcgagcgggcgctcgggctgcacgaggccgagtcgcgatcgccggtgctggccctggactcggtcgacgcggccaagcgggcggtcgcctcggggctgggcatctcgctggtgccggaggtcacggtcgccgcggagctggcggacggcaggctcagccgcatcggctggaccccgccgttccgggtgttcacccagttcgcgtggcgccaggacaactcggcgaacccgtcggtgaccgcgctggtctcggcggcggcgcaggtggtgagcgagcaggtggccgcgacacccgcgtagggcgtcgacgtgcagggtcgtggatgcggagcggccccctcgtgctgcgcagagggggccgagaccgtcggggcgacaggatttgaacctgcgaccccccgctcccaaagcgggtgcgctaccaaactgcgccacgccccggtcaccaggagcttagcgcgacgcgctaagctgttttcagcacccacccggtgggcgctgcgcgggtgtagctcaatggtagagccccagccttccaagctggtcatgcgggttcgattcccgtcacccgctccaccagatcc

SEQ ID NO: 12 - DNA, 36 bp, Artificial
  primer
tctagaccgagctggaggggggcgcagccgacccga

SEQ ID NO: 13 - DNA, 36 bp, Artificial
  primer
tctagacagatcggccagttcgcggaagttgcgttg

SEQ ID NO: 14 - DNA, 653 bp, Actinosynnema pretiosum
tctagaccgagctggaggggggcgcagccgacccgaacctgcacatcggctactgggacgcgccggactcgccaacgccacgcgcggaggcggtagtgcgcttcaccgacgaacacgtccgccgcctgcacgtgaccacgggcgaccgagtgctggacgtgggctgcggcgtaggcggcccagccctgcgcgcggtggacctgaccggcgcccacgtgaccggaatcagcatcagcgccgcccagatcacccacgcgacccacctggccaagtccgcgggccacgcggacaacaccaagttcctccacgcagacgcgatggccctcccgttcccggactcctcgttcgacgcggtcatggcgatcgagtccctgatccacatgcccgaccgcgagcgggtcctgaacgaggcaagacgcgtactgcgcccaggcgggcgactggtcctcaccgaactgttcgaacgcgccccaagacccacccgcagacacccagcgataaccgagttctgccgagcatcgatggtgtccctgcccaacgcagacgactaccccgcactactacaccgagcaggcctacgcctacgggaactcctggacatcaccgaccacaccgtccaacgcaacttccgcgaactggccgatctgtctaga

SEQ ID NO: 15 - DNA, 39 bp, Artificial
  primer
ggaagctttcggtaatggggagactcgacgccgcctgac

SEQ ID NO: 16 - DNA, 40 bp, Artificial
  primer
gggatccccgaacacccgtaaccacgcggtggcgtccccc

SEQ ID NO: 17 - DNA, 2452 bp, Actinosynnema pretiosum
ggaagctttcggtaatggggagactcgacgccgcctgacctgttgcgcatacgcaacgccttacgtcgaagcatcccccattcagctcggcgatctcaaggagaacggtgtcagcgccggacggcggacatgtcgacgacaacgacagcgagaccgaaaagatcaagaaggtcgtcggggtgcgcggcgacaagatgcgaaagctagctcagttcgctggttaccacagcgactacgccttggccgaagcgatgggcgtgcaccgctccaccgtcaccagagtccttgccggtgaacttctgcccgtcggagtccaagtggccggccgaaggcgcgtggaccgccggaacccggcgaactggcgtttccccttctgttcgccactccaatcatcgccgtcctccgggaggggcatcgatgatcaacactcttgtgcccattccgcaagagctgagattgctggacctgctgccggaccccgtggtctcgacctcgtcgttggccgcgatgctgggaatcaccgtcgaggagaccgcccaacacgcgcgggactgggtgaaccagggcgtgctggtgctgacgcagccttcgtggactgaacttcacccgaacatcacgcaccccgagttcacctcctcgcgttaccggctggccggtcgccatcgcgcgcaggagacagaccaagcccaggcactgctgcgatgggctgtgcagcaggagttcccgcaagagttggagcgcttgggcaggtacctgcatcggcgcaccttggccgcagggaaactgctcggtgatttgcgcgaggtgttcgtcgacggcttcgaggtcggggcgaagccgctgttcgacaacgccttggcggcgtacaagtggctgatcgtgcatcggcgggaagtcgtggccgtgcagcgcaccgccgcactcgtgggcgggtaccggcaagaagtgctggacatcggcgccgcgctgtggtcggtaacgctgctgaacgagcgctggagctggcgttgtgcaacaccgatcccgacagccgatcgtggtcactggccgaagcgcacggtgtcatgggatggctggagacgagcgagggcaacgcgcgggccgtgctgtggcacggtgttcgggcgttggaagccgacgaggcgcgcggggtgccggaagccgcgttggcaggcagggttgtcgggttgtcgggcacctacctgatgctcggcgatcttggccgggccgccaaggaggccaagcgggcgtgggaaatggtcgcccctgaacgggagcgacaccctctggggtacgcgacgatggtattggccatgcactgcacgcccgacgactgctctcggttctggaggcgctggcagaagatcacaaggggcggatcgacctgctcggcaagcagttccggcgggaccgcgccgcagcagcgtgaccaatcgtcgcaggcggcccgacccctttgcggccgggccgctttgcgattcgcagttcaaccctttcgcctgatcgaccttctgcacggagtcccggagtgtctcgcacacaccctgccgcgccttcccgctcaccgctgaccacctcgggccatgcgcggcccgatgtcaggacgacgtgcgcgtgcgactcgctggacgaggcgttcaccgcgctcagcgccccggatctcgattcacggcgtgctgagaaactgctccgggggatgacctcgtcccggcagcaatagggcacctccgccccgcgcaggtcaactaccaccggtgaagacacgaggccgcccccgtccgcgaggatcgcggagttgtcgtcctggtcccggctcagactgcgctccgaaaccagcaagaccttgttgcacgcctacaccgccctggcacgtctggacgaaacagtcgcgcgttccaggttgagcgccagcctgcatgaggtgaccgcgatccgtgaggctcgcgcgatcgcccacctgggcgatgcgcaccgggcggaggaaggtcaaagcgcttcccgccccgctgctcgtcgccccactgctcaacagcaccatgctgaccgccgaactcggctatacgcaccgcgccgcactcctgatcttggagcagcttgcggatagcggcctactgcgcaggtcccgccaccgcgtgtacaaccacctctacgtgcgacgggacgtgctgcctctgctcaccgaactccaccttccaagctgccccgctcagtccggaaacagtgcccccatcccagctcggccttaaggcaccgaagcgcgacaagtcgctgacctgctcaaacggcacatgatctgttggcagtcagtgggaatttgacatgcgccaggccacacggagaagagggacgcgggagtggggctgcgcgggctcgctgcgctcgccccgcagcccgcgaggcggtggcgtccgtgggggacgccaccgcgtggttacgggtgttcggggatccc

SEQ ID NO: 18 - DNA, 30 bp, Artificial
  primer
cagcaggagttcccgcaagagttggagcgc

SEQ ID NO: 19 - DNA, 40 bp, Artificial
  primer
gggatccgggaacggcctttcggggtcggcttgcgggagg

SEQ ID NO: 20 - DNA, 39 bp, Artificial
  primer
gggaattcccccggagagaaaggccgccgcagtgttcac

SEQ ID NO: 21 - DNA, 2572 bp, Actinosynnema pretiosum
gggatccgggaacggcctttcggggtcggcttgcgggaggtctgcgtggcaggaggtctggtgggacggggtcaccaggtgacgggcagctcgtagacgccgtagacgatcccgtcgtgcttgaacgggatgcgctccagcgcggtcgccagtcgcaaggtcgggacgcggcggtagagggtgttcacgacgacctgcagctccatgcgggccagcgactggcccaggcactggtgggtgccgaagctgaacgcgaagtgctgcttcaccggcctgcgcaggtccagcacgtgcgggtcgccgtcgtagacgtcggggtcgcggttgccgatcgaggtcgccagcaccacgccctcgcccgcgcggatcacctggccgtcgatctccacgtcctcggtcgccacgcggcgcatcccggtgtgcgagatcgtcaggtagcgcatcagctcctcgacggcggtctcggcgagggcggggtcgtcgcccgcctggagcagcgccagctggtcggggtgccggagcagcgcgagggtgccgagcgcgatcatgttcgcggtggtctcgtgcccgcccgccagcaccaggacggtcatgtcgaccgcctccggcctgctgatctcgcctgccagcacccgtgcgcccaggtcggagaggaggtcgtcggcggggtcggtcatcttgtcgcccaggacgcggtcgaggtggtgctggagctcctcgctggcctcggcccgttcctgcgggggcacgtcctggcggatcagcacggcgctgttgcgctcgaagacctcgtggtcggagtagggcaggcccaggacggcgctgatcgcgagggacgggatgggcagcgcgaacgcggtgaccaggtcgaccgggttggggcccgcgaggagttcgtcgatcttctcgtcgacgatccgctgcacgaacgggcgcaggcgttccaccttgcggacggtgaacgggcccaggaccatccggcgctgccggttgtgctcggggtcgtccatgagcgggaagctgctgatgaacgtcgacttgtcctcgatgcgaatcgggctggggtagccagggcgcaggaagtccgcgctgaggcgggggtcgcgcagcagctcgcgctggtgggcgtgcttggtgatcagccacggggtgctgccgtcccacaggcgcacccgcgacaggccgcccgcctcctgcgcctcgcggagtcgagggggcgggtcgaacgggtgctcgggcgacctgggcatcgggaactccggggccaggtcgggggtgggggtggtgcgggtcatcggctctcccctggtggacggtcggtgctcaccacgtgaccggcagcgagcgcaggccgaggttcatggactccagggcgaagtccaggtcctcgaacggcgtggcaggcgccaggcccggcaggccgcacagcagggtctccagcgcgatctgcagctcgacgcgcgccagcgtcatgcccacgcactggtggacgccgtagccgaacgcgaagtgccgccgggcgttgtgcctggtcacgtcgaggcggtcgggatcggggaagacgtcggggtcgcggttggccgcgggcaggttgacgatcacgccggagcccgcgggaacgagcacgccgtccagctcgacgtcgtccagggccacgcggccggtgccgtcctggacgatggtgtgccagcgcagcagctcctcgacggcgttggcgaaaccggcgggctcggcgcgcaggaaggccagctgctcggggtcgcgcaggagcgcggtcgtgcccagcgcgatcatgttcgcggtggtctcgtagcccgccagcagcacgaccatcacggtggcgacgagctgcttccgggtcagctcgccggtgcgctcgaagtccagcaccaggcggctgatcaggtcgtcgccgggggcggcccgcttggccgcgcacaggtcgtccacgaacccgaccatcgagttgatggcggcgcccatctcctcggcggacagggacgggtccagggcgcgcgtcgagtcgcgcgcgaactcgtccaggccggggcccgccacgccgagcacgccgccgatcaccagggacgggatcgggaacgcgtagtgggccaccaggtcggcgggggagcccgcctgcacgagctggcgcagctgggcgtggcagatcgtggtgatcgcggggcgcatcgcctccacggggcggctcgcgaagtgcccggcgaccatgcggcgcagcctcgcgtgctccgggtcgtccatcttgccgaagttgaacacgtcgtccgggatgtcgaacttcacgaagcgcggctgggcgcggttggcgccgtcggagctgaagcgcgggtcggtcagggcggtgcggcacacctcgtgaccggtgagcaggaacggggtgctgccgtcccacaggcggaccctggtgacgggctgctcctcccgtaattggccgtagcgcgggggtggcgcgaacgggcagtcgccccggtcccgcaggaacgggtaggcgggcaggtcggtggccacgtcctcggtgaacactgcggcggcctttctctccgggggaattccc

SEQ ID NO: 22 - DNA, 33 bp, Artificial
  primer
cctcgtggtcggagtagggcaggcccaggacgg

SEQ ID NO: 23 - DNA, 38 bp, Artificial
  primer
ggtctagaggtcacgggcggtctgcggcgaccagcagg

SEQ ID NO: 24 - DNA, 37 bp, Artificial
  primer
ggcatatgagcgacaccacgctgtccgtgcccgtccc

(19) United States
(12) Patent Application Publication
(10) Pub. No.: US 2009/0209507 A1
(43) Pub. Date: Aug. 20, 2009

(54) 15-O-Desmethylmacbecin Derivatives and Their Use in the Treatment of Cancer or B-Cell Malignancies

(76) Inventors: Sabine Gaisser, Essex (GB); Christine Martin, Essex (GB); Ming Zhang, Essex (GB); Barrie Wilkinson, Essex (GB); Nigel Coates, Essex (GB); Mohammed Nur-E-Alam, Essex (GB); William Vousden, Essex (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/294,253

(22) PCT Filed: Mar. 30, 2007
(86) PCT No.: PCT/EP2007/053131

§ 371 Date: Jan. 22, 2009

(30) Foreign Application Priority Data
Mar. 31, 2006 (GB) 0606542.9

(51) Int. Cl. A61K 31/395; C07D 225/06; A61P 35/00; C12P 17/10; C12N 5/10

(52) U.S. Cl. 514/183; 540/461; 435/121; 435/348

(57) ABSTRACT

The present invention relates to 15-O-desmethylmacbecin analogues according to the formula (IA) or (IB) herein, or a pharmaceutically acceptable salt thereof: wherein: R₁ and R₂ either both represent H or together they represent a bond (i.e. C4 to C5 is a double bond); and R₃ represents H or CONH₂ that are useful, e.g. in the treatment of cancer, B-cell malignancies, malaria, fungal infection, diseases of the central nervous system and neurodegenerative diseases, diseases dependent on angiogenesis, autoimmune diseases and/or as a prophylactic pretreatment for cancer. The present invention also provides methods for the production of these compounds and their use in medicine, in particular in the treatment and/or prophylaxis of cancer or B-cell malignancies.

### BACKGROUND OF THE INVENTION

The 90 kDa heat shock protein (Hsp90) is an abundant molecular chaperone involved in the folding and assembly of proteins, many of which are involved in signal transduction pathways (for reviews see Neckers, 2002; Sreedhar et al., 2004a; Wegele et al., 2004 and references therein). So far nearly 50 of these so-called client proteins have been identified and include steroid receptors, non-receptor tyrosine kinases e.g. src family, cyclin-dependent kinases e.g. cdk4 and cdk6, the cystic transmembrane regulator, nitric oxide synthase and others (Donzé and Picard, 1999; McLaughlin et al., 2002; Chiosis et al., 2004; Wegele et al., 2004; http://www.picard.ch/downloads/Hsp90interactors.pdf). Furthermore, Hsp90 plays a key role in stress response and protection of the cell against the effects of mutation (Bagatell and Whitesell, 2004; Chiosis et al., 2004). The function of Hsp90 is complicated and it involves the formation of dynamic multi-enzyme complexes (Bohen, 1998; Liu et al., 1999; Young et al., 2001; Takahashi et al., 2003; Sreedhar et al., 2004; Wegele et al., 2004). Hsp90 is a target for inhibitors (Fang et al., 1998; Liu et al., 1999; Blagosklonny, 2002; Neckers, 2003; Takahashi et al., 2003; Beliakoff and Whitesell, 2004; Wegele et al., 2004) resulting in degradation of client proteins, cell cycle dysregulation and apoptosis. More recently, Hsp90 has been identified as an important extracellular mediator for tumour invasion (Eustace et al., 2004). Hsp90 was identified as a new major therapeutic target for cancer therapy which is mirrored in the intense and detailed research about Hsp90 function (Blagosklonny et al., 1996; Neckers, 2002; Workman and Kaye, 2002; Beliakoff and Whitesell, 2004; Harris et al., 2004; Jez et al., 2003; Lee et al., 2004) and the development of high-throughput screening assays (Carreras et al., 2003; Rowlands et al., 2004). Hsp90 inhibitors include compound classes such as ansamycins, macrolides, purines, pyrazoles, coumarin antibiotics and others (for review see Bagatell and Whitesell, 2004; Chiosis et al., 2004 and references therein).

The benzenoid ansamycins are a broad class of chemical structures characterised by an aliphatic ring of varying length joined either side of an aromatic ring structure. Naturally occurring ansamycins include: macbecin and 18,21-dihydromacbecin (also known as macbecin I and macbecin II respectively) (1 & 2; Tanida et al., 1980), geldanamycin (3; DeBoer et al., 1970; DeBoer and Dietz, 1976; WO 03/106653 and references therein), and the herbimycin family (4; 5, 6, Omura et al., 1979, Iwai et al., 1980 and Shibata et al, 1986a, WO 03/106653 and references therein).

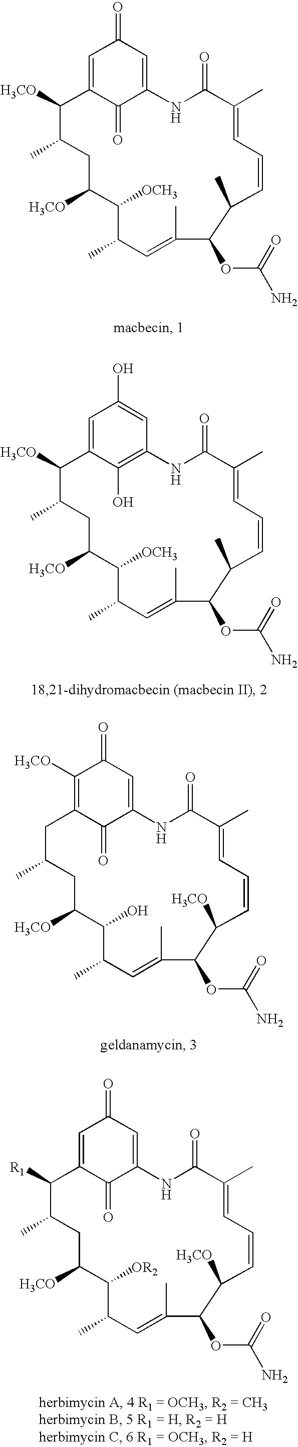

Ansamycins were originally identified for their antibacterial and antiviral activity, however, recently their potential utility as anticancer agents has become of greater interest (Beliakoff and Whitesell, 2004). Many Hsp90 inhibitors are currently being assessed in clinical trials (Csermely and Soti, 2003; Workman, 2003). In particular, geldanamycin has nanomolar potency and apparent specificity for aberrant protein kinase dependent tumour cells (Chiosis et al., 2003; Workman, 2003).

It has been shown that treatment with Hsp90 inhibitors enhances the induction of tumour cell death by radiation and increased cell killing abilities (e.g. breast cancer, chronic myeloid leukaemia and non-small cell lung cancer) by combination of Hsp90 inhibitors with cytotoxic agents has also been demonstrated (Neckers, 2002; Beliakoff and Whitesell, 2004). The potential for anti-angiogenic activity is also of interest: the Hsp90 client protein HIF-1α plays a key role in the progression of solid tumours (Hur et al., 2002; Workman and Kaye, 2002; Kaur et al., 2004).

Hsp90 inhibitors also function as immunosuppressants and are involved in the complement-induced lysis of several types of tumour cells after Hsp90 inhibition (Sreedhar et al., 2004). Treatment with Hsp90 inhibitors can also result in induced superoxide production (Sreedhar et al., 2004a) associated with immune cell-mediated lysis (Sreedhar et al., 2004). The use of Hsp90 inhibitors as potential anti-malaria drugs has also been discussed (Kumar et al., 2003). Furthermore, it has been shown that geldanamycin interferes with the formation of complex glycosylated mammalian prion protein PrP^{c} (Winklhofer et al., 2003).

As described above, ansamycins are of interest as potential anticancer and anti-B-cell malignancy compounds, however the currently available ansamycins exhibit poor pharmacological or pharmaceutical properties, for example they show poor water solubility, poor metabolic stability, poor bioavailability or poor formulation ability (Goetz et al., 2003; Workman 2003; Chiosis 2004). Both herbimycin A and geldanamycin were identified as poor candidates for clinical trials due to their strong hepatotoxicity (review Workman, 2003) and geldanamycin was withdrawn from Phase I clinical trials due to hepatotoxicity (Supko et al., 1995; WO 03/106653).

Geldanamycin was isolated from culture filtrates of Streptomyces hygroscopicus and shows strong activity in vitro against protozoa and weak activity against bacteria and fungi. In 1994 the association of geldanamycin with Hsp90 was shown (Whitesell et al., 1994). The biosynthetic gene cluster for geldanamycin was cloned and sequenced (Allen and Ritchie, 1994; Rascher et al., 2003; WO 03/106653). The DNA sequence is available under the NCBI accession number AY179507. The isolation of genetically engineered geldanamycin producer strains derived from S. hygroscopicus subsp. duamyceticus JCM4427 and the isolation of 4,5-dihydro-7-O-descarbamoyl-7-hydroxygeldanamycin and 4,5-dihydro-7-O-descarbamoyl-7-hydroxy-17-O-demethylgeldanamycin were described recently (Hong et al., 2004). By feeding geldanamycin to the herbimycin producing strain Streptomyces hygroscopicus AM-3672 the compounds 15-hydroxygeldanamycin, the tricyclic geldanamycin analogue KOSN-1633 and methyl-geldanamycinate were isolated (Hu et al., 2004). The two compounds 17-formyl-17-demethoxy-18-O-21-O-dihydrogeldanamycin and 17-hydroxymethyl-17-demethoxygeldanamycin were isolated from S. hygroscopicus K279-78. S. hygroscopicus K279-78 is S. hygroscopicus NRRL 3602 containing cosmid pKOS279-78 which has a 44 kbp insert which contains various genes from the herbimycin producing strain Streptomyces hygroscopicus AM-3672 (Hu et al., 2004). Substitutions of acyltransferase domains have been made in four of the modules of the polyketide synthase of the geldanamycin biosynthetic cluster (Patel et al., 2004). AT substitutions were carried out in modules 1, 4 and 5 leading to the fully processed analogues 14-desmethyl-geldanamycin, 8-desmethyl-geldanamycin and 6-desmethoxy-geldanamycin and the not fully processed 4,5-dihydro-6-desmethoxy-geldanamycin. Substitution of the module 7 AT lead to production of three 2-desmethyl compounds, KOSN1619, KOSN1558 and KOSN1559, one of which (KOSN1559), a 2-demethyl-4,5-dihydro-17-demethoxy-21-deoxy derivative of geldanamycin, binds to Hsp90 with a 4-fold greater binding affinity than geldanamycin and an 8-fold greater binding affinity than 17-AAG. However this is not reflected in an improvement in the IC₅₀ measurement using SKBr₃. Another analogue, a novel nonbenzoquinoid geldanamycin, designated KOS-1806 has a monophenolic structure (Rascher et al., 2005). No activity data was given for KOS-1806.

In 1979 the ansamycin antibiotic herbimycin A was isolated from the fermentation broth of Streptomyces hygroscopicus strain No. AM-3672 and named according to its potent herbicidal activity. The antitumour activity was established by using cells of a rat kidney line infected with a temperature sensitive mutant of Rous sarcoma virus (RSV) for screening for drugs that reverted the transformed morphology of the these cells (for review see Uehara, 2003). Herbimycin A was postulated as acting primarily through the binding to Hsp90 chaperone proteins but the direct binding to the conserved cysteine residues and subsequent inactivation of kinases was also discussed (Uehara, 2003).

Chemical derivatives have been isolated and compounds with altered substituents at C19 of the benzoquinone nucleus and halogenated compounds in the ansa chain showed less toxicity and higher antitumour activities than herbimycin A (Omura et al., 1984; Shibata et al., 1986b). The sequence of the herbimycin biosynthetic gene cluster was identified in WO 03/106653 and in a recent paper (Rascher et al., 2005).

The ansamycin compounds macbecin (1) and 18,21-dihydromacbecin (2) (C-14919E-1 and C-14919E-1), identified by their antifungal and antiprotozoal activity, were isolated from the culture supernatants of Nocardia sp No. C-14919 (Actinosynnema pretiosum subsp pretiosum ATCC 31280) (Tanida et al., 1980; Muroi et al., 1980; Muroi et al., 1981; U.S. Pat. No. 4,315,989 and U.S. Pat. No. 4,187,292). 18,21-Dihydromacbecin is characterized by containing the dihydroquinone form of the nucleus. Both macbecin and 18,21-dihydromacbecin were shown to possess similar antibacterial and antitumour activities against cancer cell lines such as the murine leukaemia P388 cell line (Ono et al., 1982). Reverse transcriptase and terminal deoxynucleotidyl transferase activities were not inhibited by macbecin (Ono et al., 1982). The Hsp90 inhibitory function of macbecin has been reported in the literature (Bohen, 1998; Liu et al., 1999). The conversion of macbecin and 18,21-dihydromacbecin after adding to a microbial culture broth into a compound with a hydroxy group instead of a methoxy group at a certain position or positions is described in U.S. Pat. No. 4,421,687 and U.S. Pat. No. 4,512,975.

During a screen of a large variety of soil microorganisms, the compounds TAN-420A to E were identified from producer strains belonging to the genus Streptomyces (7-11, EP 0 110 710).

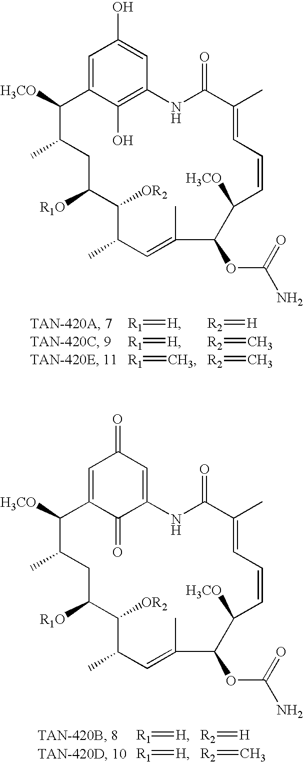

In 2000, the isolation of the geldanamycin related, non-benzoquinone ansamycin metabolite reblastin from cell cultures of Streptomyces sp. S6699 and its potential therapeutic value in the treatment of rheumatoid arthritis was described (Stead et al., 2000).

A further Hsp90 inhibitor, distinct from the chemically unrelated benzoquinone ansamycins is Radicicol (monorden) which was originally discovered for its antifungal activity from the fungus Monosporium bonorden (for review see Uehara, 2003) and the structure was found to be identical to the 14-membered macrolide isolated from Nectria radicicola. In addition to its antifungal, antibacterial, anti-protozoan and cytotoxic activity it was subsequently identified as an inhibitor of Hsp90 chaperone proteins (for review see Uehara, 2003; Schulte et al., 1999). The anti-angiogenic activity of radicicol (Hur et al., 2002) and semi-synthetic derivates thereof (Kurebayashi et al., 2001) has also been described.

Recent interest has focussed on 17-amino derivatives of geldanamycin as a new generation of ansamycin anticancer compounds (Bagatell and Whitesell, 2004), for example 17-(allylamino)-17-desmethoxy geldanamycin (17-AAG, 12) (Hostein et al., 2001; Neckers, 2002; Nimmanapalli et al., 2003; Vasilevskaya et al., 2003; Smith-Jones et al., 2004) and 17-desmethoxy-17-N,N-dimethylaminoethylamino-geldanamycin (17-DMAG, 13) (Egorin et al., 2002; Jez et al., 2003). More recently geldanamycin was derivatised on the 17-position to create 17-geldanamycin amides, carbamates, ureas and 17-arylgeldanamycin (Le Brazidec et al., 2003). A library of over sixty 17-alkylamino-17-demethoxygeldanamycin analogues has been reported and tested for their affinity for Hsp90 and water solubility (Tian et al., 2004). A further approach to reduce the toxicity of geldanamycin is the selective targeting and delivering of an active geldanamycin compound into malignant cells by conjugation to a tumour-targeting monoclonal antibody (Mandler et al., 2000).

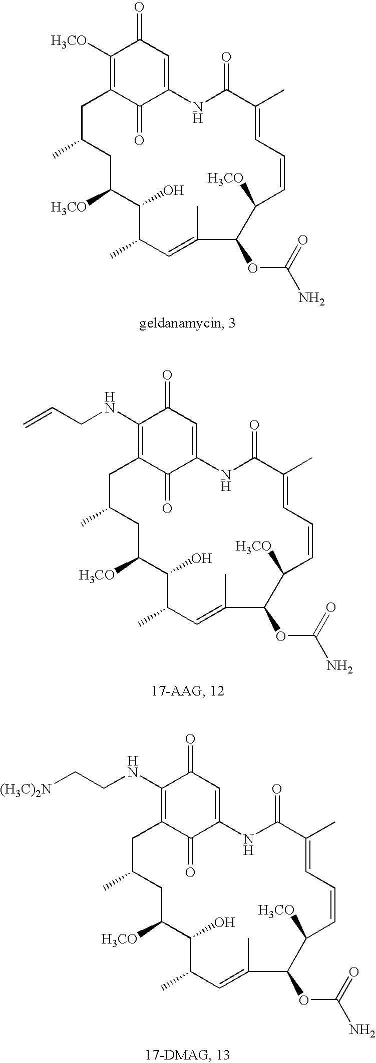

Whilst many of these derivatives exhibit reduced hepatotoxicity they still have only limited water solubility. For example 17-AAG requires the use of a solubilising carrier (e.g. Cremophore®, DMSO-egg lecithin), which itself may result in side-effects in some patients (Hu et al., 2004).

Most of the ansamycin class of Hsp90 inhibitors bear the common structural moiety: the benzoquinone which is a Michael acceptor that can readily form covalent bonds with nucleophiles such as proteins, glutathione, etc. The benzoquinone moiety also undergoes redox equilibrium with dihydroquinone, during which oxygen radicals are formed, which give rise to further unspecific toxicity (Dikalov et al., 2002). For example treatment with geldanamycin can result in induced superoxide production (Sreedhar et al., 2004a).

Therefore, there remains a need to identify novel ansamycin derivatives, which may have utility in the treatment of cancer and/or B-cell malignancies, preferably such ansamycins have improved water solubility, an improved pharmacological profile and/or reduced side-effect profile for administration. The present invention discloses novel ansamycin analogues generated by genetic engineering of the parent producer strain. In particular the present invention discloses novel 15-O-desmethylmacbecin analogues, which generally have improved pharmaceutical properties compared with the presently available ansamycins; in particular they are expected show improvements in respect of one or more of the following properties: activity against different cancer sub-types, toxicity, water solubility, metabolic stability, bioavailability and formulation ability. Preferably the 15-O-desmethylmacbecin analogues show improved water solubility and/or bioavailability.

### SUMMARY OF THE INVENTION

The inventors of the present invention have made significant effort to clone and elucidate the gene cluster that is responsible for the biosynthesis of macbecin. With this insight, the gene that is responsible for the addition of a methyl group to the oxygen attached at the C15 position has been specifically targeted, e.g. by integration into mbcMT2, targeted deletion of a region of the macbecin cluster including all or part of the mbcMT2 gene optionally followed by insertion of gene(s) or other methods of rendering MbcMT2 non-functional e.g. chemical inhibition, site-directed mutagenesis of mbcMT2 or mutagenesis of the cell for example by the use of UV radiation, in order to produce novel derivatives devoid of a methyl group at the C15 position. As a result, the present invention provides 15-O-desmethylmacbecin analogues, methods for the preparation of these compounds, and methods for the use of these compounds in medicine or as intermediates in the production of further compounds.

Therefore, in a first aspect the present invention provides analogues of macbecin which are lacking the methyl group usually attached to the oxygen attached to the C15 position, the macbecin analogues may either have a benzoquinone (i.e. they are macbecin I analogues) or have a dihydroquinone moiety (i.e., they are 18,21-dihydromacbecin or macbecin II analogues).

In a more specific aspect the present invention provides 15-O-desmethylmacbecin analogues according to the formula (IA) or (IB) below, or a pharmaceutically acceptable salt thereof:

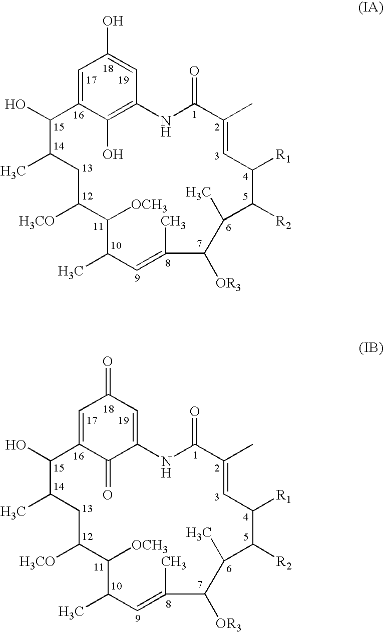

wherein:

R₁ and R₂ either both represent H or together they represent a bond (i.e. C4 to C5 is a double bond); and

R₃ represents H or CONH₂.

15-O-Desmethylmacbecin analogues are also referred to herein as “compounds of the invention”, such terms are used interchangeably herein. Compounds of formula (IA) and (IB) are referred to collectively in the foregoing as compounds of formula (I).

The above structure shows a representative tautomer and the invention embraces all tautomers of the compounds of formula (I) for example keto compounds where enol compounds are illustrated and vice versa.

The invention embraces all stereoisomers of the compounds defined by structure (I) as shown above.

In a further aspect, the present invention provides 15-O-desmethylmacbecin analogues such as compounds of formula (I) or a pharmaceutically acceptable salt thereof, for use as a pharmaceutical.

### DEFINITIONS

The articles “a” and “an” are used herein to refer to one or to more than one (i.e. at least one) of the grammatical objects of the article. By way of example “an analogue” means one analogue or more than one analogue.

As used herein the term “analogue(s)” refers to chemical compounds that are structurally similar to another but which differ slightly in composition (as in the replacement of one atom by another or in the presence or absence of a particular functional group).

As used herein, the term “homologue(s)” refers a homologue of a gene or of a protein encoded by a gene disclosed herein from either an alternative macbecin biosynthetic cluster from a different macbecin producing strain or a homologue from an alternative ansamycin biosynthetic gene cluster e.g. from geldanamycin, herbimycin or reblastatin. Such homologue(s) encode a protein that performs the same function of can itself perform the same function as said gene or protein in the synthesis of macbecin or a related ansamycin polyketide. Preferably, such homologue(s) have at least 40% sequence identity, preferably at least 60%, at least 70%, at least 80%, at least 90% or at least 95% sequence identity to the sequence of the particular gene disclosed herein (Table 3, SEQ ID NO: 11 which is a sequence of all the genes in the cluster, from which the sequences of particular genes may be deduced). Percentage identity may be calculated using any program known to a person of skill in the art such as BLASTn or BLASTp, available on the NCBI website.

As used herein, the term “cancer” refers to a benign or malignant new growth of cells in skin or in body organs, for example but without limitation, breast, prostate, lung, kidney, pancreas, brain, stomach or bowel. A cancer tends to infiltrate into adjacent tissue and spread (metastasise) to distant organs, for example to bone, liver, lung or the brain. As used herein the term cancer includes both metastatic tumour cell types, such as but not limited to, melanoma, lymphoma, leukaemia, fibrosarcoma, rhabdomyosarcoma, and mastocytoma and types of tissue carcinoma, such as but not limited to, colorectal cancer, prostate cancer, small cell lung cancer and non-small cell lung cancer, breast cancer, pancreatic cancer, bladder cancer, renal cancer, gastric cancer, gliobastoma, primary liver cancer and ovarian cancer.

As used herein the term “B-cell malignancies” includes a group of disorders that include chronic lymphocytic leukaemia (CLL), multiple myeloma, and non-Hodgkin's lymphoma (NHL). They are neoplastic diseases of the blood and blood forming organs. They cause bone marrow and immune system dysfunction, which renders the host highly susceptible to infection and bleeding.

As used herein, the term “bioavailability” refers to the degree to which or rate at which a drug or other substance is absorbed or becomes available at the site of biological activity after administration. This property is dependent upon a number of factors including the solubility of the compound, rate of absorption in the gut, the extent of protein binding and metabolism etc. Various tests for bioavailability that would be familiar to a person of skill in the art are described in Egorin et al. (2002).

The term “water solubility” as used in this application refers to solubility in aqueous media, e.g. phosphate buffered saline (PBS) at pH 7.3. An exemplary water solubility assay is given in the Examples below.

As used herein the term “post-PKS genes(s)” refers to the genes required for post-polyketide synthase modifications of the polyketide, for example but without limitation monooxygenases, O-methyltransferases and carbamoyltransferases. Specifically, in the macbecin system these modifying genes include mbcM, mbcN, mbcP, mbcMT1, mbcMT2 and mbcP450.

The pharmaceutically acceptable salts of compounds of the invention such as the compounds of formula (I) include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. References hereinafter to a compound according to the invention include both compounds of formula (I) and their pharmaceutically acceptable salts.

As used herein the terms “18,21-dihydromacbecin” and “macbecin II” (the dihydroquinone form of macbecin) are used interchangeably.

### BRIEF DESCRIPTION OF THE DRAWINGS

FIG. 1: Representation of the biosynthesis of macbecin showing the first putative enzyme free intermediate, pre-macbecin and the post-PKS processing to macbecin. The list of PKS processing steps in the figure in not intended to represent the order of events. The following abbreviations are used for particular genes in the cluster: AL0—AHBA loading domain; ACP—Acyl Carrier Protein; KS—β-ketosynthase; AT—acyl transferase; DH—dehydratase; ER—enoyl reductase; KR—β-ketoreductase.

FIG. 2: Depiction of the sites of post-PKS processing of pre-macbecin to give macbecin.

FIG. 3: Sequence of the amplified PCR product PCRgp25 (SEQ ID NO: 14)

FIG. 4: Diagrammatic representation of generation of the engineered strain BIOT-3819 in which plasmid pGP25 was integrated into the chromosome by homologous recombination resulting in mbcMT2 gene disruption.

FIG. 5: Diagrammatic representation of the generation of an Actinosynnema pretiosum strain carrying a deletion of the methyltransferase genes mbcMT1 and mbcMT2 and subsequently complemented by mbcMT1.

FIG. 6: Sequence of amplified PCR product PCR1 (SEQ ID NO: 17)

FIG. 7: Sequence of amplified PCR product PCR 2 (SEQ ID NO: 21)

FIG. 8: Sequence of 831 bp amplified PCR product mbcMT1 (SEQ ID NO: 25)

FIG. 9: Structures of the compounds (14-15) produced in the examples

### DESCRIPTION OF THE INVENTION

The present invention provides 15-O-desmethylmacbecin analogues, as set out above, methods for the preparation of these compounds, methods for the use of these compounds in medicine and the use of these compounds as intermediates or templates for further semi-synthetic derivatisation or derivatisation by biotransformation methods.

Preferably the 15-O-desmethylmacbecin analogues have a structure according to Formula IA.

Preferably the 15-O-desmethylmacbecin analogues have a structure according to Formula IB.

Preferably R₃ represents —CONH₂

Preferably R₁ and R₂ together represent a bond

In one preferred embodiment of the invention R₁ and R₂ together represent a bond and R₃ represents CONH₂.

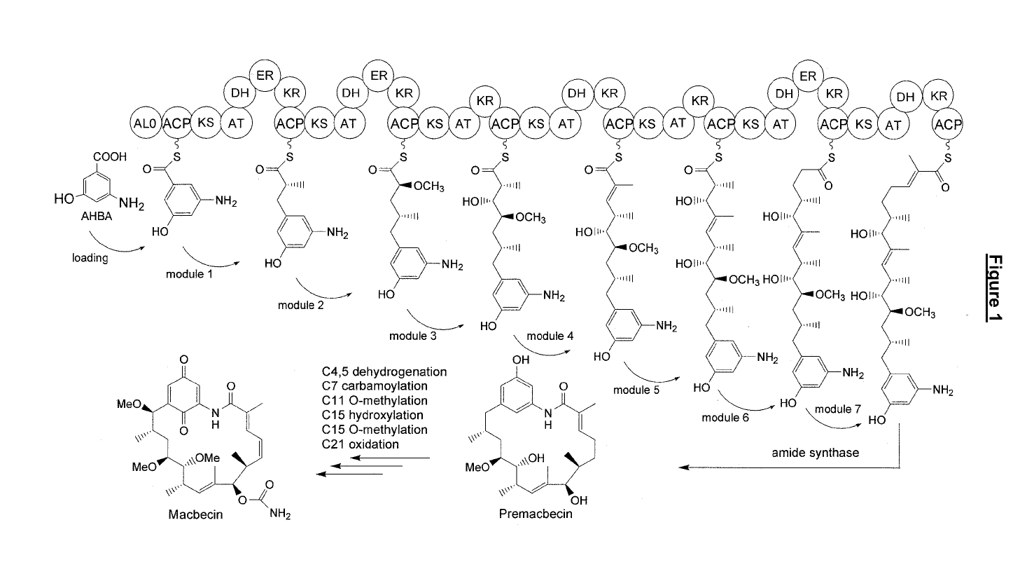

The preferred stereochemistry of the non-hydrogen sidechains to the ansa ring is as shown in FIGS. 1, 2 and 9 below (that is to say the preferred stereochemistry follows that of macbecin).

The compounds of the invention may be isolated from the fermentation broth in their benzoquinone form or in their dihydroquinone form. It is well-known in the art that benzoquinones can be chemically converted to dihydroquinones (reduction) and vice versa (oxidation), therefore these forms may be readily interconverted using methods well-known to a person of skill in the art. For example, but without limitation, if the benzoquinone form is isolated then it may be converted to the corresponding dihydroquinones. As an example (but not by way of limitation) this may be achieved in organic media with a source of hydride, such as but not limited to, LiAlH₄ or SnCl₂—HCl. Alternatively this transformation may be mediated by dissolving the benzoquinone form of the compound of the invention in organic media and then washing with an aqueous solution of a reducing agent, such as, but not limited to, sodium hydrosulfite (Na₂S₂O₄ or sodium thionite). Preferably, this transformation is carried out by dissolving the compound of the invention in ethyl acetate and mixing this solution vigorously with an aqueous solution of sodium hydrosulfite (Muroi et al., 1980). The resultant organic solution can then be washed with water, dried and the solvent removed under reduced pressure to yield an almost quantitative amount of the 18,21-dihydro form of the compound of the invention.

In order to oxidise a dihydroquinone to a quinone several routes are available, including, but not limited to the following: the dihydroquinone form of the compound of the invention is dissolved in an organic solvent such as ethyl acetate and then this solution is vigorously mixed with an aqueous solution of iron (III) chloride (FeCl₃). The organic solution can then be washed with water, dried and the organic solvent removed under reduced pressure to yield an almost quantitative amount of the benzoquinone form of the macbecin compound.

The present invention also provides a pharmaceutical composition comprising a 15-O-desmethylmacbecin analogue, or a pharmaceutically acceptable salt thereof, together with a pharmaceutically acceptable carrier.

The present invention also provides for the use of a 15-O-desmethylmacbecin analogue as a substrate for further modification either by biotransformation or by synthetic chemistry.

In one aspect the present invention provides for the use of a 15-O-desmethylmacbecin analogue in the manufacture of a medicament. In a further embodiment the present invention provides for the use of a 15-O-desmethylmacbecin analogue in the manufacture of a medicament for the treatment of cancer and/or B-cell malignancies. In a further embodiment the present invention provides for the use of an 15-O-desmethylmacbecin analogue in the manufacture of a medicament for the treatment of malaria, fungal infection, diseases of the central nervous system, diseases dependent on angiogenesis, autoimmune diseases and/or as a prophylactic pre-treatment for cancer.

In another aspect the present invention provides for the use of a 15-O-desmethylmacbecin analogue in medicine. In a further embodiment the present invention provides for the use of a 15-O-desmethylmacbecin analogue in the treatment of cancer and/or B-cell malignancies. In a further embodiment the present invention provides for the use of a 15-O-desmethylmacbecin analogue in the manufacture of a medicament for the treatment of malaria, fungal infection, diseases of the central nervous system and neurodegenerative diseases, diseases dependent on angiogenesis, autoimmune diseases and/or as a prophylactic pre-treatment for cancer.

In a further embodiment the present invention provides a method of treatment of cancer and/or B-cell malignancies, said method comprising administering to a patient in need thereof a therapeutically effective amount of a 15-O-desmethylmacbecin analogue. In a further embodiment the present invention provides a method of treatment of malaria, fungal infection, diseases of the central nervous system and neurodegenerative diseases, diseases dependent on angiogenesis, autoimmune diseases and/or a prophylactic pre-treatment for cancer, said method comprising administering to a patient in need thereof a therapeutically effective amount of a 15-O-desmethylmacbecin analogue.

As noted above, compounds of the invention may be expected to be useful in the treatment of cancer and/or B-cell malignancies. Compounds of the invention may also be effective in the treatment of other indications for example, but not limited to malaria, fungal infection, diseases of the central nervous system and neurodegenerative diseases, diseases dependent on angiogenesis, autoimmune diseases such as rheumatoid arthritis or as a prophylactic pre-treatment for cancer.

Diseases of the central nervous system and neurodegenerative diseases include, but are not limited to, Alzheimer's disease, Parkinson's disease, Huntington's disease, prion diseases, spinal and bulbar muscular atrophy (SBMA) and amyotrophic lateral sclerosis (ALS).

Diseases dependent on angiogenesis include, but are not limited to, age-related macular degeneration, diabetic retinopathy and various other ophthalmic disorders, atherosclerosis and rheumatoid arthritis.

Autoimmune diseases include, but are not limited to, rheumatoid arthritis, multiple sclerosis, type I diabetes, systemic lupus erythematosus and psoriasis.

“Patient” embraces human and other animal (especially mammalian) subjects, preferably human subjects. Accordingly the methods and uses of the 15-O-desmethylmacbecin analogues of the invention are of use in human and veterinary medicine, preferably human medicine.

The aforementioned compounds of the invention or a formulation thereof may be administered by any conventional method for example but without limitation they may be administered parenterally (including intravenous administration), orally, topically (including buccal, sublingual or transdermal), via a medical device (e.g. a stent), by inhalation, or via injection (subcutaneous or intramuscular). The treatment may consist of a single dose or a plurality of doses over a period of time.

Whilst it is possible for a compound of the invention to be administered alone, it is preferable to present it as a pharmaceutical formulation, together with one or more acceptable carriers. Thus there is provided a pharmaceutical composition comprising a compound of the invention together with one or more pharmaceutically acceptable diluents or carriers. The diluents(s) or carrier(s) must be “acceptable” in the sense of being compatible with the compound of the invention and not deleterious to the recipients thereof. Examples of suitable carriers are described in more detail below.

The compounds of the invention may be administered alone or in combination with other therapeutic agents. Co-administration of two (or more) agents may allow for significantly lower doses of each to be used, thereby reducing the side effects seen. It might also allow resensitisation of a disease, such as cancer, to the effects of a prior therapy to which the disease has become resistant. There is also provided a pharmaceutical composition comprising a compound of the invention and a further therapeutic agent together with one or more pharmaceutically acceptable diluents or carriers.

In a further aspect, the present invention provides for the use of a compound of the invention in combination therapy with a second agent e.g. a second agent for the treatment of cancer or B-cell malignancies such as a cytotoxic or cytostatic agent.

In one embodiment, a compound of the invention is co-administered with another therapeutic agent e.g. a therapeutic agent such as a cytotoxic or cytostatic agent for the treatment of cancer or B-cell malignancies. Exemplary further agents include cytotoxic agents such as alkylating agents and mitotic inhibitors (including topoisomerase II inhibitors and tubulin inhibitors). Other exemplary further agents include DNA binders, antimetabolites and cytostatic agents such as protein kinase inhibitors and tyrosine kinase receptor blockers. Suitable agents include, but are not limited to, methotrexate, leukovorin, prenisone, bleomycin, cyclophosphamide, 5-fluorouracil, paclitaxel, docetaxel, vincristine, vinblastine, vinorelbine, doxorubicin (adriamycin), tamoxifen, toremifene, megestrol acetate, anastrozole, goserelin, anti-HER2 monoclonal antibody (e.g. trastuzumab, trade name Herceptin™), capecitabine, raloxifene hydrochloride, EGFR inhibitors (e.g. gefitinib, trade name Iressa®, erlotinib, trade name Tarceva™, cetuximab, trade name Erbitux™), VEGF inhibitors (e.g. bevacizumab, trade name Avastin™), proteasome inhibitors (e.g. bortezomib, trade name Velcade™) or imatinib, trade name Glivec®. Further suitable agents include, but are not limited to, conventional chemotherapeutics such as cisplatin, cytarabine, cyclohexylchloroethylnitrosurea, gemcitabine, Ifosfamid, leucovorin, mitomycin, mitoxantone, oxaliplatin, taxanes including taxol and vindesine; hormonal therapies; monoclonal antibody therapies such as cetuximab (anti-EGFR); protein kinase inhibitors such as dasatinib, lapatinib; histone deacetylase (HDAC) inhibitors such as vorinostat; angiogenesis inhibitors such as sunitinib, sorafenib, lenalidomide; and mTOR inhibitors such as temsirolimus. Additionally, a compound of the invention may be administered in combination with other therapies including, but not limited to, radiotherapy or surgery.

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatine and acacia. Additionally, lubricating agents such as magnesium stearate, stearic acid, glyceryl behenate and talc may be included.

Solid compositions of a similar type may also be employed as fillers in gelatine capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerine, and combinations thereof.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatine, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations suitable for topical administration in the mouth include lozenges comprising the active ingredient in a flavoured basis, usually sucrose and acacia or tragacanth; pastilles comprising the active ingredient in an inert basis such as gelatine and glycerine, or sucrose and acacia; and mouth-washes comprising the active ingredient in a suitable liquid carrier.

It should be understood that in addition to the ingredients particularly mentioned above the formulations of this invention may include other agents conventional in the art having regard to the type of formulation in question, for example those suitable for oral administration may include flavouring agents.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base.

Alternatively, the active agent may be formulated in a cream with an oil-in-water cream base or a water-in-oil base.

For parenteral administration, fluid unit dosage forms are prepared utilizing the active ingredient and a sterile vehicle, for example but without limitation water, alcohols, polyols, glycerine and vegetable oils, water being preferred. The active ingredient, depending on the vehicle and concentration used, can be either suspended or dissolved in the vehicle. In preparing solutions the active ingredient can be dissolved in water for injection and filter sterilised before filling into a suitable vial or ampoule and sealing.

Advantageously, agents such as local anesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Parenteral suspensions are prepared in substantially the same manner as solutions, except that the active ingredient is suspended in the vehicle instead of being dissolved and sterilization cannot be accomplished by filtration. The active ingredient can be sterilised by exposure to ethylene oxide before suspending in the sterile vehicle. Advantageously, a surfactant or wetting agent is included in the composition to facilitate uniform distribution of the active ingredient.

The compounds of the invention may also be administered using medical devices known in the art. For example, in one embodiment, a pharmaceutical composition of the invention can be administered with a needleless hypodermic injection device, such as the devices disclosed in U.S. Pat. No. 5,399,163; U.S. Pat. No. 5,383,851; U.S. Pat. No. 5,312,335; U.S. Pat. No. 5,064,413; U.S. Pat. No. 4,941,880; U.S. Pat. No. 4,790,824; or U.S. Pat. No. 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

The dosage to be administered of a compound of the invention will vary according to the particular compound, the disease involved, the subject, and the nature and severity of the disease and the physical condition of the subject, and the selected route of administration. The appropriate dosage can be readily determined by a person skilled in the art.

The compositions may contain from 0.1% by weight, preferably from 5-60%, more preferably from 10-30% by weight, of a compound of invention, depending on the method of administration.

It will be recognized by one of skill in the art that the optimal quantity and spacing of individual dosages of a compound of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

In a further aspect the present invention provides methods for the production of 15-O-desmethylmacbecin analogues.

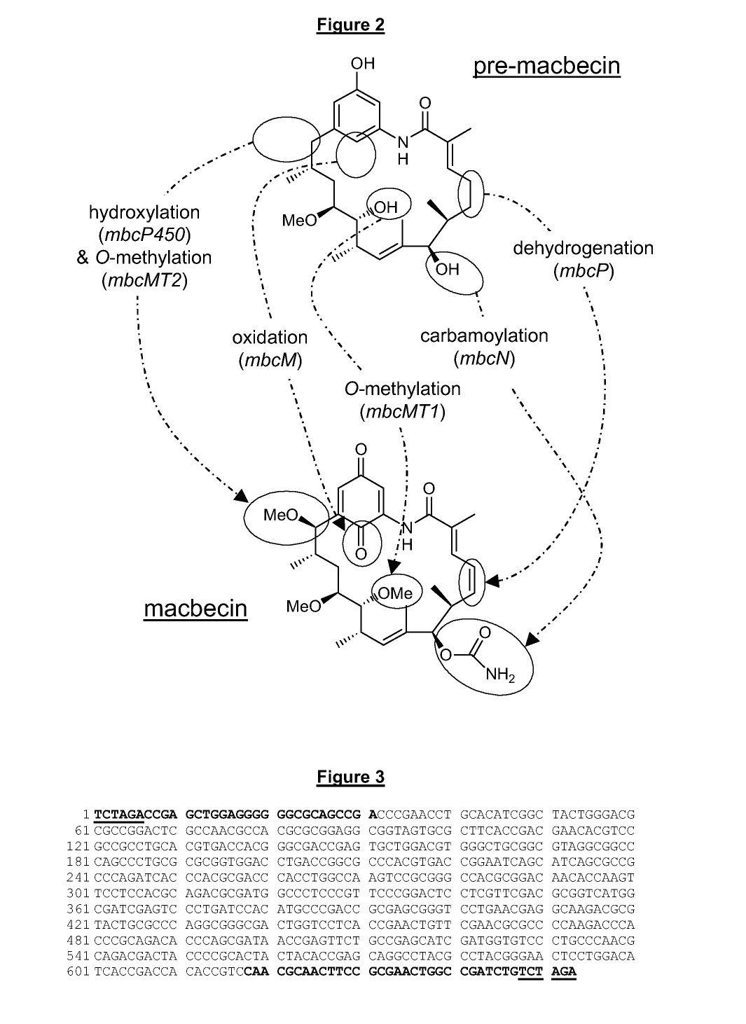

Macbecin can be considered to be biosynthesised in two stages. In the first stage the core-PKS genes assemble the macrolide core by the repeated assembly of 2-carbon units which are then cyclised to form the first enzyme-free intermediate “pre-macbecin”, see FIG. 1. In the second stage a series of “post-PKS” tailoring enzymes (e.g. P450 monooxygenases, methyltransferases, FAD-dependent oxygenases and a carbamoyltransferase) act to add the various additional groups to the pre-macbecin template resulting in the final parent compound structure, see FIG. 2. The 15-O-desmethylmacbecin analogues may be biosynthesised in a similar manner.

This biosynthetic production may be exploited by genetic engineering of suitable producer strains to result in the production of novel compounds. In particular, the present invention provides a method of producing 15-O-desmethylmacbecin analogues said method comprising:
a) providing a first host strain that produces macbecin or an analogue thereof when cultured under appropriate conditions
b) deleting or inactivating one or more post-PKS genes, wherein at least one of the post-PKS genes is mbcMT2, or a homologue thereof
c) culturing said modified host strain under suitable conditions for the production of 15-O-desmethylmacbecin analogues; and
d) optionally isolating the compounds produced.

In step (a) by “macbecin or an analogue thereof” is meant macbecin or those analogues of macbecin that are embraced by the definitions of R₁-R₃.

In step (b), deleting or inactivating one or more post-PKS genes, wherein at least one of the post-PKS genes is mbcMT2, or a homologue thereof will suitably be done selectively.

In a further embodiment, step b) comprises inactivating mbcMT2 (or a homologue thereof) by integration of DNA into the mbcMT2 gene (or a homologue thereof) such that functional MbcMT2 protein is not produced. In an alternative embodiment, step b) comprises making a targeted deletion of the mbcMT2 gene, or a homologue thereof. In a further embodiment mbcMT2, or a homologue thereof, is inactivated by site-directed mutagenesis. In a further embodiment the host strain of step a) is subjected to mutagenesis and a modified strain is selected in which one or more of the post-PKS enzymes is not functional, wherein at least one of these is MbcMT2. The present invention also encompasses mutations of the regulators controlling the expression of mbcMT2, or a homologue thereof, a person of skill in the art will appreciate that deletion or inactivation of a regulator may have the same outcome as deletion or inactivation of the gene.

In a further embodiment the strain of step b) is complemented with one or more of the genes that have been deleted or inactivated, not including mbcMT2 or a homologue thereof.

In a particular embodiment of the present invention, a method of selectively deleting or inactivating a post PKS gene comprises:
(i) designing degenerate oligos based on homologue(s) of the gene of interest (e.g. from the rifamycin biosynthetic cluster and/or other available sequences of O-methyl transferases) and isolating the internal fragment of the gene of interest (e.g. mbcMT2) from a suitable macbecin producing strain, by using these primers in a PCR reaction;
(ii) introduction of a plasmid containing this fragment into either the same, or a different macbecin producing strain followed by homologous recombination, which results in the disruption of the targeted gene (e.g. mbcMT2 or a homologue thereof),
(iii) culturing the strain thus produced under conditions suitable for the production of the macbecin analogues, i.e. 15-O-desmethylmacbecin analogues.
   In a specific embodiment, the macbecin-producing strain in step (i) is Actinosynnema mirum (A. mirum). In a further specific embodiment the macbecin-producing strain in step (ii) is Actinosynnema pretiosum (A. pretiosum).

A person of skill in the art will appreciate that an equivalent strain may be achieved using alternative methods to that described above, e.g.:
Degenerate oligos may be used to amplify the gene of interest from any macbecin producing strain for example, but not limited to A. pretiosum, or A. mirum
Different degenerate oligos may be designed which will successfully amplify an appropriate region of the mcbMT2 gene or a homologue thereof of a macbecin producer, or a homologue thereof.
The sequence of the mbcMT2 gene of the A. pretiosum strain may be used to generate the oligos which may be specific to the mbcMT2 gene of A. pretiosum and then the internal fragment may be amplified from any macbecin producing strain e.g A. pretiosum or A. mirum.
The sequence of the mbcMT2 gene of the A. pretiosum strain may be used along with the sequence of homologous genes to generate degenerate oligos to the mbcMT2 gene of A. pretiosum and then the internal fragment may be amplified from any macbecin producing strain e.g A. pretiosum or A. mirum.

In further aspects of the invention, additional post-PKS genes may also be deleted or inactivated in addition to mbcMT2. FIG. 2 shows the activity of the post-PKS genes in the macbecin biosynthetic cluster. A person of skill in the art would thus be able to identify which additional post-PKS genes would need to be deleted or inactivated in order to arrive at a strain that will produce the compound(s) of interest.

In further aspects of the invention, an engineered strain in which one or more post-PKS genes including mbcMT2 have been deleted or inactivated as above, has re-introduced into it one or more of the same post PKS genes not including mbcMT2, or homologues thereof, e.g. from an alternative macbecin producing strain, or even from the same strain.

It may be observed in these systems that when a strain is generated in which MbcMT2, or a homologue thereof, does not function as a result of one of the methods described including inactivation or deletion, that more than one macbecin analogue may be produced. There are a number of possible reasons for this which will be appreciated by those skilled in the art. For example there may be a preferred order of post-PKS steps and removing a single activity leads to all subsequent steps being carried out on substrates that are not natural to the enzymes involved. This can lead to intermediates building up in the culture broth due to a lowered efficiency towards the novel substrates presented to the post-PKS enzymes, or to shunt products which are no longer substrates for the remaining enzymes possibly because the order of steps has been altered.

A person of skill in the art will appreciate that the ratio of compounds observed in a mixture can be manipulated by using variations in the growth conditions.

When a mixture of compounds is observed these can be readily separated using standard techniques some of which are described in the following examples.

15-O-Desmethylmacbecin analogues may be screened by a number of methods, as described herein, and in the circumstance where a single compound shows a favourable profile a strain can be engineered to make this compound preferably. In the unusual circumstance when this is not possible, an intermediate can be generated which is then biotransformed to produce the desired compound.

The present invention provides novel macbecin analogues generated by the selected deletion or inactivation of one or more post-PKS genes from the macbecin PKS gene cluster. In particular, the present invention relates to novel 15-O-desmethylmacbecin analogues produced by the selected deletion or inactivation of at least mbcMT2, or a homologue thereof, from the macbecin PKS gene cluster. In one embodiment, mbcMT2, or a homologue thereof, alone is deleted or inactivated. In an alternative embodiment, other post-PKS genes in addition to mbcMT2 are additionally deleted or inactivated. In a specific embodiment, additional genes selected from the group consisting of: mbcM, mbcN, mbcP, mbcMT1 and mbcP450 are deleted or inactivated in the host strain. In a further embodiment, additionally 1 or more of the post-PKS genes selected from the group consisting of: mbcM, mbcN, mbcP, mbcMT1 and mbcP450 are deleted or inactivated. In a further embodiment, additionally 2 or more of the post-PKS genes selected from the group consisting of mbcM, mbcN, mbcP, mbcMT1 and mbcP450 are deleted or inactivated. In a further embodiment, additionally 3 or more of the post-PKS genes selected from the group consisting of mbcM, mbcN, mbcP, mbcMT1 and mbcP450 are deleted or inactivated. In a further embodiment, additionally 4 or more of the post-PKS genes selected from the group consisting of mbcM, mbcN, mbcP, mbcMT1 and mbcP450 are deleted or inactivated.

A person of skill in the art will appreciate that a gene does not need to be completely deleted for it to be rendered non-functional, consequentially the term “deleted or inactivated” as used herein encompasses any method by which a gene is rendered non-functional including but not limited to: deletion of the gene in its entirety, deletion of part of the gene, inactivation by insertion into the target gene, site-directed mutagenesis which results in the gene either not being expressed or being expressed in an inactive form, mutagenesis of the host strain which results in the gene either not being expressed or being expressed in an inactive form (e.g. by radiation or exposure to mutagenic chemicals, protoplast fusion or transposon mutagenesis). Alternatively the function of an active gene can be impaired chemically with inhibitors, for example metapyrone (alternative name 2-methyl-1,2-di(3-pyridyl-1-propanone), EP 0 627 009) and ancymidol are inhibitors of oxygenases and these compounds can be added to the production medium to generate analogues. Additionally, sinefungin is a methyl transferase inhibitor that can be used similarly but for the inhibition of methyl transferase activity in vivo (McCammon and Parks 1981).

In an alternative embodiment, there is provided a method for the production of a 15-O-desmethylmacbecin analogue, said method comprising:
a) providing a first host strain that produces macbecin when cultured under appropriate conditions
b) deleting or inactivating one or more post-PKS genes, wherein at least one of the post-PKS genes is mbcMT2, or a homologue thereof,
c) re-introducing some or all of the post-PKS genes not including mbcMT2, or a homologue thereof,
d) culturing said modified host strain under suitable conditions for the production of 15-O-desmethylmacbecin analogues; and
e) optionally isolating the compounds produced.

In a further embodiment an engineered strain in which one or more post-PKS genes including mbcMT2 have been deleted or inactivated is complemented by one or more of the post PKS genes from a heterologous PKS cluster including, but not limited to the clusters directing the biosynthesis of rifamycin, ansamitocin, geldanamycin or herbimycin.

In an alternative embodiment, all of the post-PKS genes may be deleted or inactivated and then one or more of the genes, but not including mbcMT2, or a homologue thereof, may then be reintroduced by complementation (e.g. at an attachment site, on a self-replicating plasmid or by insertion into a homologous region of the chromosome). Therefore, in a particular embodiment the present invention relates to methods for the generation of 15-O-desmethylmacbecin analogues, said method comprising:
a) providing a first host strain that produces macbecin when cultured under appropriate conditions
b) selectively deleting or inactivating all the post-PKS genes,
c) culturing said modified host strain under suitable conditions for the production of novel compounds; and
d) optionally isolating the compounds produced.

In an alternative embodiment, one or more of the deleted post-PKS genes are reintroduced, provided that mbcMT2 is not one of the genes reintroduced. In a further embodiment, 1 or more of the post-PKS genes selected from the group consisting of mbcM, mbcN, mbcP, mbcMT1 and mbcP450 are reintroduced. In a further embodiment, 2 or more of the post-PKS genes selected from the group consisting of mbcM, mbcN, mbcP, mbcMT1 and mbcP450 are reintroduced. In a further embodiment, 3 or more of the post-PKS genes selected from the group consisting of mbcM, mbcN, mbcP, mbcMT1 and mbcP450 are reintroduced. In a further embodiment, 4 or more of the post-PKS genes selected from the group consisting of mbcM, mbcN, mbcP, mbcMT1 and mbcP450 are reintroduced. In a further alternative embodiment, mbcM, mbcN, mbcP, mbcMT1 and mbcP450 are reintroduced.

Additionally, it will be apparent to a person of skill in the art that a subset of the post-PKS genes, including mbcMT2, or a homologue thereof, could be deleted or inactivated and a smaller subset of said post-PKS genes not including mbcMT2 could be reintroduced to arrive at a strain producing 15-O-desmethylmacbecin analogues.

In a specific embodiment, mbcMT1 and mbcMT2 are deleted and mbcMT1 is reintroduced under a suitable promoter at an attachment site to yield 15-O-desmethylmacbecin analogues.

A person of skill in the art will appreciate that there are a number of ways to generate a strain that contains the biosynthetic gene cluster for macbecin but that is lacking at least mbcMT2, or a homologue thereof, or lacking at least the function of mbcMT2 or homologue thereof.

It is well known to those skilled in the art that polyketide gene clusters may be expressed in heterologous hosts (Pfeifer and Khosla, 2001). Accordingly, the present invention includes the transfer of the macbecin biosynthetic gene cluster without mbcMT2 or with a non-functional mutant of mbcMT2, with or without resistance and regulatory genes, either otherwise complete or containing additional deletions, into a heterologous host. Methods and vectors for the transfer as defined above of such large pieces of DNA are well known in the art (Rawlings, 2001; Staunton and Weissman, 2001) or are provided herein in the methods disclosed. In this context a preferred host cell strain is a prokaryote, more preferably an actinomycete or Escherichia coli, still more preferably include, but are not limited to Actinosynnema mirum (A. mirum), Actinosynnema pretiosum subsp. pretiosum (A. pretiosum), S. hygroscopicus, S. hygroscopicus sp., S. hygroscopicus var. ascomyceticus, Streptomyces tsukubaensis, Streptomyces coelicolor, Streptomyces lividans, Saccharopolyspora erythraea, Streptomyces fradiae, Streptomyces avermitilis, Streptomyces cinnamonensis, Streptomyces rimosus, Streptomyces albus, Streptomyces griseofuscus, Streptomyces longisporoflavus, Streptomyces venezuelae, Streptomyces albus, Micromonospora sp., Micromonospora griseorubida, Amycolatopsis mediterranei or Actinoplanes sp. N902-109. Further examples include Streptomyces hygroscopicus subsp. geldanus and Streptomyces violaceusniger.

In one embodiment the entire biosynthetic cluster without mbcMT2 is transferred. In an alternative embodiment the entire PKS is transferred without any of the associated post-PKS genes, including mbcMT2.

In a further embodiment the entire macbecin biosynthetic cluster is transferred and then manipulated according to the description herein.

In an alternative aspect of the invention, the 15-O-desmethylmacbecin analogue of the present invention may be further processed by biotransformation with an appropriate strain. The appropriate strain either being an available wild type strain for example, but without limitation Actinosynnema mirum, Actinosynnema pretiosum subsp. pretiosum, S. hygroscopicus, S. hygroscopicus sp. Alternatively, an appropriate strain may be a engineered to allow biotransformation with particular post-PKS enzymes for example, but without limitation, those encoded by mbcM, mbcN, mbcP, mbcMT1, mbcP450 (as defined herein), gdmN, gdmM, gdmL, gdmP, (Rascher et al., 2003) the geldanamycin O-methyl transferase, hbmN, hbmL, hbmP, (Rascher et al., 2005) herbimycin O-methyl transferases and further herbimycin mono-oxygenases, asm7, asm10, asm11, asm12, asm19 and asm21 (Cassady et al., 2004, Spiteller et al., 2003). Where genes have yet to be identified or the sequences are not in the public domain it is routine to those skilled in the art to acquire such sequences by standard methods. For example the sequence of the gene encoding the geldanamycin O-methyl transferase is not in the public domain, but one skilled in the art could generate a probe, either a heterologous probe using a similar O-methyl transferase, or a homologous probe by designing degenerate primers based on sequences from available homologous genes to carry out amplification of an O-methyl transferase fragment. Either type of probe can then be used in Southern Blot analysis on a geldanamycin producing strain and thus acquire this gene to generate biotransformation systems.

In a particular embodiment the strain may have had one or more of its native polyketide clusters deleted, either entirely or in part, or otherwise inactivated, so as to prevent the production of the polyketide produced by said native polyketide cluster. Said engineered strain may be selected from the group including, for example but without limitation, Actinosynnema mirum, Actinosynnema pretiosum subsp. pretiosum, S. hygroscopicus, S. hygroscopicus sp., S. hygroscopicus var. ascomyceticus, Streptomyces tsukubaensis, Streptomyces coelicolor, Streptomyces lividans, Saccharopolyspora erythraea, Streptomyces fradiae, Streptomyces avermitilis, Streptomyces cinnamonensis, Streptomyces rimosus, Streptomyces albus, Streptomyces griseofuscus, Streptomyces longisporoflavus, Streptomyces venezuelae, Micromonospora sp., Micromonospora griseorubida, Amycolatopsis mediterranei or Actinoplanes sp. N902-109. Further possible strains include Streptomyces hygroscopicus subsp. geldanus and Streptomyces violaceusniger.

In a further aspect the present invention provides host strains which naturally produce macbecin or analogue thereof, in which the mbcMT2 gene, or a homologue thereof, has been deleted or inactivated such that it thereby produces 15-O-desmethylmacbecin or an analogue thereof (e.g. a 15-O-desmethylmacbecin analogue as defined by compounds of formula (I)) and their use in the production of 15-O-desmethylmacbecin or analogues thereof.

Therefore, in one embodiment the present invention provides a genetically engineered strain which naturally produces macbecin in its unaltered state, said strain having one or more post-PKS genes from the macbecin PKS gene cluster deleted wherein one of said deleted or inactivated post-PKS genes is mbcMT2, or a homologue thereof.

The invention embraces all products of the inventive processes described herein.

Although the process for preparation of the 15-O-desmethylmacbecin analogues of the invention as described above is substantially or entirely biosynthetic, it is not ruled out to produce or interconvert 15-O-desmethylmacbecin analogues of the invention by a process which comprises standard synthetic chemical methods.

In order to allow for the genetic manipulation of the macbecin PKS gene cluster, first the gene cluster was sequenced from Actinosynnema pretiosum subsp. pretiosum however, a person of skill in the art will appreciate that there are alternative strains which produce macbecin, for example but without limitation Actinosynnema mirum. The macbecin biosynthetic gene cluster from these strains may be sequenced as described herein for Actinosynnema pretiosum subsp. pretiosum, and the information used to generate equivalent strains.

Further aspects of the invention include:
An engineered strain based on a macbecin producing strain in which mbcMT2 and optionally further post-PKS genes have been deleted or inactivated, particularly such an engineered strain in which mbcMT2 has been deleted or inactivated or such an engineered strain in which mbcMT1 and mbcMT2 have been deleted or inactivated and mbcMT1 has been reintroduced. Suitably the macbecin producing strain is A. pretiosum or A. mirum.
A process for producing an 15-O-desmethylmacbecin analogue which comprises culturing an aforementioned strain. The strains will be cultured in suitable media known to a skilled person and provided with suitable feed materials eg appropriate starter acids.
Such a process further comprising the step of isolating 15-O-desmethylmacbecin or an analogue thereof. Isolation may be performed by conventional means eg chromatography (eg HPLC).
Use of such an engineered strain in the preparation of a 15-O-desmethylmacbecin analogue.

Compounds of the invention are advantageous in that they may be expected to have one or more of the following properties: good activity against one or more different cancer sub-types compared with the parent compound; good toxicological profile such as good hepatotoxicity profile, good nephrotoxicity, good cardiac safety; good water solubility; good metabolic stability; good formulation ability; good bioavailability; good pharmacokinetic or pharmacodynamic properties such as tight binding to Hsp90, fast on-rate of binding to Hsp90 and/or good brain pharmacokinetics; good cell uptake; and low binding to erythrocytes.

### EXAMPLES

### General Methods

### Fermentation of Cultures

Conditions used for growing the bacterial strains Actinosynnema pretiosum subsp. pretiosum ATCC 31280 (U.S. Pat. No. 4,315,989) and Actinosynnema mirum DSM 43827 (KCC A-0225, Watanabe et al., 1982) were described in the U.S. Pat. No. 4,315,989 and U.S. Pat. No. 4,187,292. Methods used herein were adapted from these patents and are as follows for culturing of broths in tubes or flasks in shaking incubators, variations to the published protocols are indicated in the examples. Strains were grown on ISP2 agar (Medium 3, Shirling, E. B. and Gottlieb, D., 1966) at 28° C. for 2-3 days and used to inoculate seed medium (Medium 1, see below adapted from U.S. Pat. No. 4,315,989 and U.S. Pat. No. 4,187,292). The inoculated seed medium was then incubated with shaking between 200 and 300 rpm with a 5 or 2.5 cm throw at 28° C. for 48 h. For production of macbecin, 18,21-dihydromacbecin and macbecin analogues such as 15-O-desmethylmacbecin analogues the fermentation medium (Medium 2, see below and U.S. Pat. No. 4,315,989 and U.S. Pat. No. 4,187,292) was inoculated with 2.5%-10% of the seed culture and incubated with shaking between 200 and 300 rpm with a 5 or 2.5 cm throw initially at 28° C. for 24 h followed by 26° C. for four to six days. The culture was then harvested for extraction.

### Media

### Medium 1—Seed Medium

In 1 L of distilled water

| | | |
|---|---|---|
| Glucose | 20 | g |
| Soluble potato starch (Sigma) | 30 | g |
| Spray dried corn steep liquor (Roquette Freres) | 10 | g |
| ‘Nutrisoy’ toasted soy flour (Archer Daniels Midland) | 10 | g |
| Peptone from milk solids (Sigma) | 5 | g |
| NaCl | 3 | g |
| CaCO₃ | 5 | g |

| | |
|---|---|
| Adjust pH with NaOH | 7.0 |

Sterilisation by autoclaving at 121° C. for 20 minutes.
Apramycin was added when appropriate after autoclaving to give a final concentration of 50 mg/L.

### Medium 2—Fermentation Medium

In 1 L of distilled water

| | | |
|---|---|---|
| Glycerol | 50 | g |
| Spray dried corn steep liquor (Roquette Freres) | 10 | g |
| ‘Bacto’ yeast extract (Difco) | 20 | g |
| KH₂PO₄ | 20 | g |
| MgCl₂•6H₂O | 5 | g |
| CaCO₃ | 1 | g |

| | |
|---|---|
| Adjust pH with NaOH | 6.5 |

Sterilisation by autoclaving at 121° C. for 20 minutes.

### Medium 3—ISP2 Medium

In 1 L of distilled water

| | | |
|---|---|---|
| Malt extract | 10 | g |
| Yeast extract | 4 | g |
| Dextrose | 4 | g |
| Agar | 15 | g |

| | |
|---|---|
| Adjust pH with NaOH | 7.3 |

Sterilisation by autoclaving at 121° C. for 20 minutes.

### Medium 4—MAM

In 1 L of distilled water

| | | |
|---|---|---|
| Wheat starch | 10 | g |
| Corn steep solids | 2.5 | g |
| Yeast extract | 3 | g |
| CaCO₃ | 3 | g |
| Iron sulphate | 0.3 | g |
| Agar | 20 | g |

Sterilisation by autoclaving at 121° C. for 20 minutes.

### Extraction of Culture Broths for LCMS Analysis

Culture broth (1 mL) and ethyl acetate (1 mL) was added and mixed for 15-30 min followed by centrifugation for 10 min. 0.5 mL of the organic layer was collected, evaporated to dryness and then re-dissolved in 0.23 mL of methanol and 0.02 mL of a 1% solution of FeCl₃.

### LCMS Analysis Procedures

### Method 1

LCMS was performed using an integrated Agilent HP1100 HPLC system in combination with a Bruker Daltonics Esquire 3000+electrospray mass spectrometer operating in positive and/or negative ion mode. Chromatography was achieved over a Phenomenex Hyperclone column (C₁₈ BDS, 3u, 150×4.6 mm) eluting over 11 min at a flow rate of 1 mL/min with a linear gradient from acetonitrile+0.1% formic acid/water+0.1% formic acid (40/60) to acetonitrile+0.1% formic acid/water+0.1% formic acid (80/20). UV spectra were recorded between 190 and 400 nm, with extracted chromatograms taken at 210, 254 and 276 nm. Mass spectra were recorded between 100 and 1500 amu.

### Method 2

LCMS was performed using an Agilent HP1100 HPLC system in combination with a Bruker Daltonics Esquire 3000+electrospray mass spectrometer operating in positive and/or negative ion mode. Chromatography was achieved over a Phenomenex Hyperclone column (C₁₈ BDS, 3u, 150×4.6 mm) eluting at a flow rate of 1 mL/min using the following gradient elution process; T=0, 10% B; T=2, 10% B; T=20, 100% B; T=22, 100% B; T=22.05, 10% B; T=25, 10% B. Mobile phase A=water+0.1% formic acid; mobile phase B=acetonitrile+0.1% formic acid. UV spectra were recorded between 190 and 400 nm, with extracted chromatograms taken at 210, 254 and 276 nm. Mass spectra were recorded between 100 and 1500 amu.

### NMR Structure Elucidation Methods

NMR spectra were recorded on a Bruker Advance 500 spectrometer at 298 K operating at 500 MHz and 125 MHz for ¹H and ¹³C respectively. Standard Bruker pulse sequences were used to acquire ¹H-¹H COSY, APT, HMBC and HMQC spectra. NMR spectra were referenced to the residual proton or standard carbon resonances of the solvents in which they were run.

### Assessment of Compound Purity

Purified compounds were analysed using LCMS method 2 described above. Purity was assessed by MS and at multiple wavelengths (210, 254 & 276 nm). All compounds were >95% pure at all wavelengths. Purity was finally confirmed by inspection of the ¹H and ¹³C NMR spectra.

### Assessment of Water Solubility

Water solubility may be tested as follows: A 10 mM stock solution of the 15-O-desmethylmacbecin analogue is prepared in 100% DMSO at room temperature. Triplicate 0.01 mL aliquots are made up to 0.5 mL with either 0.1 M PBS, pH 7.3 solution or 100% DMSO in amber vials. The resulting 0.2 mM solutions are shaken in the dark, at room temperature on an IKA® vibrax VXR shaker for 6 h, followed by transfer of the resulting solutions or suspensions into 2 mL Eppendorf tubes and centrifugation for 30 min at 13200 rpm. Aliquots of the supernatant fluid are then analysed by LCMS as described above.

Compounds are quantified by peak area measurement at 258 nm. All analyses are performed in triplicate and the solubility of the 15-O-desmethylmacbecin compounds calculated by comparing PBS solutions with 0.2 mM in DMSO (with an assumed solubility of 100% in DMSO).

### In Vitro Bioassay for Anticancer Activity

In vitro evaluation of compounds for anticancer activity in a panel of human tumour cell lines in a monolayer proliferation assay was carried out at the Oncotest Testing Facility, Institute for Experimental Oncology, Oncotest GmbH, Freiburg. The characteristics of the selected cell lines are summarised in Table 1.

TABLE 1
Test cell lines

| # | Cell line | Characteristics |
|---|---|---|
| 1 | CNXF 498NL | CNS |
| 2 | CXF HT29 | Colon |
| 3 | LXF 1121L | Lung, large cell ca |
| 4 | MCF-7 | Breast, NCI standard |
| 5 | MEXF 394NL | Melanoma |
| 6 | DU145 | Prostate - PTEN positive |

The Oncotest cell lines were established from human tumor xenografts as described by Roth et al., (1999). The origin of the donor xenografts was described by Fiebig et al., (1999). Other cell lines are either obtained from the NCI (DU145, MCF-7) or purchased from DSMZ, Braunschweig, Germany.

All cell lines, unless otherwise specified, were grown at 37° C. in a humidified atmosphere (95% air, 5% CO₂) in a ‘ready-mix’ medium containing RPMI 1640 medium, 10% fetal calf serum, and 0.1 mg/mL gentamicin (PAA, Cölbe, Germany).

A modified propidium iodide assay was used to assess the effects of the test compound(s) on the growth of the human tumour cell lines (Dengler et al., (1995)).

Briefly, cells were harvested from exponential phase cultures by trypsinization, counted and plated in 96 well flat-bottomed microtitre plates at a cell density dependent on the cell line (5-10.000 viable cells/well). After 24 h recovery to allow the cells to resume exponential growth, 0.010 mL of culture medium (6 control wells per plate) or culture medium containing a 15-O-desmethylmacbecin analogue were added to the wells. Each concentration was plated in triplicate. Compounds are applied in two concentrations (1 μg/mL and 10 μg/mL). Following 4 days of continuous exposure, cell culture medium with or without test compound was replaced by 0.2 mL of an aqueous propidium iodide (PI) solution (7 mg/L). To measure the proportion of living cells, cells were permeabilized by freezing the plates. After thawing the plates, fluorescence was measured using the Cytofluor 4000 microplate reader (excitation 530 nm, emission 620 nm), giving a direct relationship to the total number of viable cells.

Growth inhibition is expressed as treated/control×100 (% T/C).

### Example 1

### Sequencing of the Macbecin PKS Gene Cluster

Genomic DNA was isolated from Actinosynnema pretiosum (ATCC 31280) and Actinosynnema mirum (DSM 43827, ATCC 29888) using standard protocols described in Kieser et al., (2000) DNA sequencing was carried out by the sequencing facility of the Biochemistry Department, University of Cambridge, Tennis Court Road, Cambridge CB2 1QW using standard procedures.

Primers BIOSG104 5′-GGTCTAGAGGTCAGTGCCCCCGCGTACCGTCGT-3′ (SEQ ID NO: 1) AND BIOSG105 5′-GGCATATGCTTGTGCTCGGGCTCAAC-3′ (SEQ ID NO: 2) were employed to amplify the carbamoyltransferase-encoding gene gdmN from the geldanamycin biosynthetic gene cluster of Streptomyces hygroscopicus NRRL 3602 (Accession number of sequence: AY179507) using standard techniques. Southern blot experiments were carried out using the DIG Reagents and Kits for Non-Radioactive Nucleic Acid Labelling and Detection according to the manufacturers' instructions (Roche). The DIG-labeled gdmN DNA fragment was used as a heterologous probe. Using the gdmN generated probe and genomic DNA isolated from A. pretiosum 2112 an approximately 8 kb EcoRI fragment was identified in Southern Blot analysis. The fragment was cloned into Litmus 28 applying standard procedures and transformants were identified by colony hybridization. The clone p3 was isolated and the approximately 7.7 kb insert was sequenced. DNA isolated from clone p3 was digested with EcoRI and EcoRI/SacI and the bands at around 7.7 kb and at about 1.2 kb were isolated, respectively. Labelling reactions were carried out according to the manufacturers' protocols. Cosmid libraries of the two strains named above were created using the vector SuperCos 1 and the Gigapack III XL packaging kit (Stratagene) according to the manufacturers' instructions. These two libraries were screened using standard protocols and as a probe, the DIG-labelled fragments of the 7.7 kb EcoRI fragment derived from clone p3 were used. Cosmid 52 was identified from the cosmid library of A. pretiosum and submitted for sequencing to the sequencing facility of the Biochemistry Department of the University of Cambridge. Similarly, cosmid 43 and cosmid 46 were identified from the cosmid library of A. mirum. All three cosmids contain the 7.7 kb EcoRI fragment as shown by Southern Blot analysis.

An around 0.7 kbp fragment of the PKS region of cosmid 43 was amplified using primers BIOSG124 5′-CCCGCCCGCGCGAGCGGCGCGTGGCCGCCCGAGGGC-3′ (SEQ ID NO: 3) and BIOSG125 5′-GCGTCCTCGCGCAGCCACGCCACCAGCAGCTCCAGC-3′ (SEQ ID NO: 4) applying standard protocols, cloned and used as a probe for screening the A. pretiosum cosmid library for overlapping clones. The sequence information of cosmid 52 was also used to create probes derived from DNA fragments amplified by primers BIOSG130 5′-CCAACCCCGCCGCGTCCCCGGCCGCGCCGAACACG-3′ (SEQ ID NO: 5) and BIOSG131 5′-GTCGTCGGCTACGGGCCGGTGGGGCAGCTGCTGT-5′ (SEQ ID NO: 6) as well as BIOSG132 5′-GTCGGTGGACTGCCCTGCGCCTGATCGCCCTGCGC-3′ (SEQ ID NO: 7) and BIOSG133 5′-GGCCGGTGGTGCTGCCCGAGGACGGGGAGCTGCGG-3′ (SEQ ID NO: 8) which were used for screening the cosmid library of A. pretiosum. Cosmids 311 and 352 were isolated and cosmid 352 was sent for sequencing. Cosmid 352 contains an overlap of approximately 2.7 kb with cosmid 52. To screen for further cosmids, an approximately 0.6 kb PCR fragment was amplified using primers BIOSG136 5′-CACCGCTCGCGGGGGTGGCGCGGCGCACGACGTGG CTGC-3′ (SEQ ID NO: 9) and BIOSG137 5′-CCTCCTCGGACAGCGCGATCAGCGCCGCGC ACAGCGAG-3′ (SEQ ID NO: 10) and cosmid 311 as template applying standard protocols. The cosmid library of A. pretiosum was screened and cosmid 410 was isolated. It overlaps approximately 17 kb with cosmid 352 and was sent for sequencing. The sequence of the three overlapping cosmids (cosmid 52, cosmid 352 and cosmid 410) was assembled. The sequenced region spans about 100 kbp and 23 open reading frames were identified potentially constituting the macbecin biosynthetic gene cluster, (SEQ ID NO: 11). The location of each of the open reading frames within SEQ ID NO: 11 is shown in Table 3

TABLE 2
Summary of the cosmids

| Cosmid | Strain |
|---|---|
| Cosmid 43 | Actinosynnema mirum ATCC 29888 |
| Cosmid 46 | Actinosynnema mirum ATCC 29888 |
| Cosmid 52 | Actinosynnema pretiosum ATCC 31280 |
| Cosmid 311 | Actinosynnema pretiosum ATCC 31280 |
| Cosmid 352 | Actinosynnema pretiosum ATCC 31280 |
| Cosmid 410 | Actinosynnema pretiosum ATCC 31280 |

TABLE 3
location of each of the open reading
frames within SEQ ID NO: 11

| Nucleotide position in | | Function of the encoded |
|---|---|---|
| SEQ ID NO: 11 | Gene Name | protein |
| 14925-17909* | mbcRII | transcriptional regulator |
| 18025-19074c | mbcO | aminohydroquinate synthase |
| 19263-20066c* | mbc? | unknown, AHBA biosynthesis |
| 20330-40657 | mbcAI | PKS |
| 40654-50859 | mbcAII | PKS |
| 50867-62491* | mbcAIII | PKS |
| 62500-63276* | mbcF | amide synthase |
| 63281-64852* | mbcM | C21 monooxygenase |
| 64899-65696c* | PH | phosphatase |
| 65693-66853c* | OX | oxidoreductase |
| 66891-68057c* | Ahs | AHBA synthase |
| 68301-68732* | Adh | ADHQ dehydratase |
| 68690-69661c* | AHk | AHBA kinase |
| 70185-72194c* | mbcN | carbamoyltransferase |
| 72248-73339c | mbcH | methoxymalonyl ACP pathway |
| 73336-74493c | mbcI | methoxymalonyl ACP pathway |
| 74490-74765c | mbcJ | methoxymalonyl ACP pathway |
| 74762-75628c* | mbcK | methoxymalonyl ACP pathway |
| 75881-76537 | mbcG | methoxymalonyl ACP pathway |
| 76534-77802* | mbcP | C4,5 monooxygenase |
| 77831-79054* | mbcP450 | P450 |
| 79119-79934* | mbcMT1 | O-methyltransferase |
| 79931-80716* | mbcMT2 | O-methyltransferase |

[Note 1:
c indicates that the gene is encoded by the complement DNA strand;
Note 2:
it is sometimes the case that more than one potential candidate start codon can been identified. One skilled in the art will recognise this and be able to identify alternative possible start codons. We have indicated those genes which have more than one possible start codon with a ‘*’ symbol. Throughout we have indicated what we believe to be the start codon, however, a person of skill in the art will appreciate that it may be possible to generate active protein using an alternative start codon.]

### Example 2

### Generation of Strain BIOT-3819: an Actinosynnema Pretiosum Strain in which the Methyltransferase mbcMT2 has been Interrupted by Insertion of a Plasmid

2.1. Construction of Plasmid pGP25

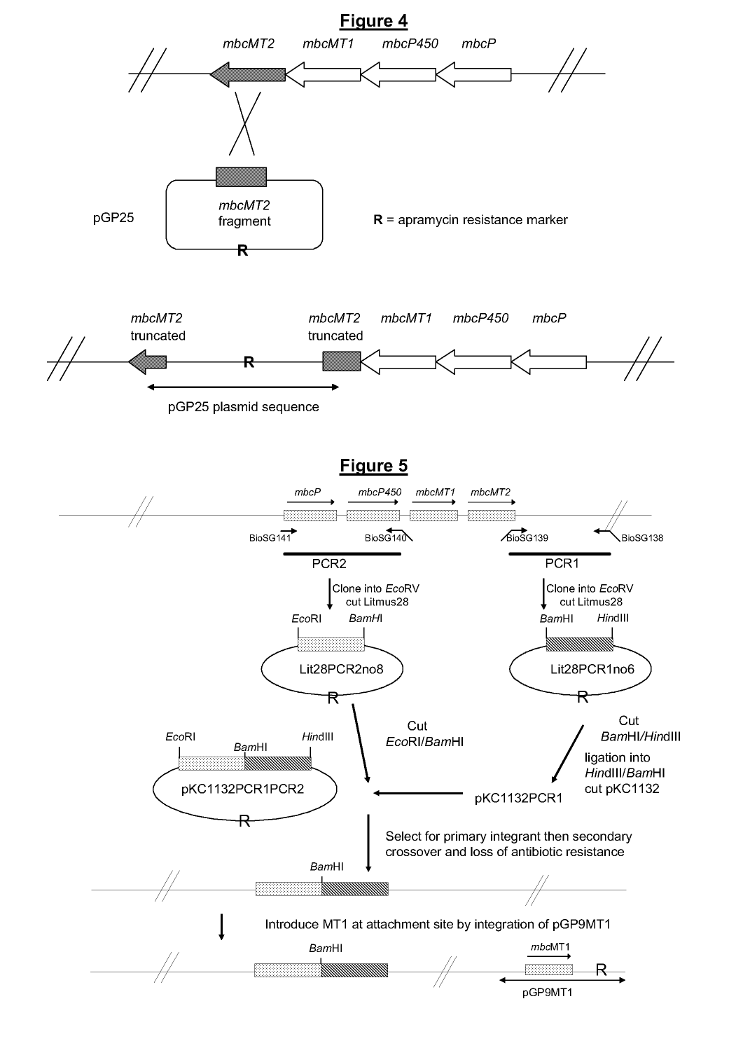

Oligos gpMT2a TCTAGACCGAGCTGGAGGGGGGCGCAGCCGACCCGA (SEQ ID NO: 12) and gpMT2b TCTAGACAGATCGGCCAGTTCGCGGAAGTTGCGTTG (SEQ ID NO: 13) were used to amplify a 653 bp region of DNA from Actinosynnema pretiosum (ATCC 31280) in a standard PCR reaction using cosmid 52 (from example 1) as the template and Pfu DNA polymerase. A 5′ extension was designed in each oligo to introduce an XbaI site (underlined) to aid cloning of the amplified fragment. The amplified PCR product (PCRgp25, SEQ ID NO: 14, FIG. 3) encoded a truncated form of mbcMT2 with 46 bp deleted from the 5′ end of the gene and 87 bp deleted from the 3′ end of the gene. This 653 bp fragment was cloned into pUC19 that had been linearised with SmaI, resulting in plasmid pGP23. The 647 bp XbaI fragment of pGP23 was ligated into XbalI cut pKC1132. The resultant plasmid, designated pGP25, is apramycin resistant and contains an internal fragment of the A. pretiosum mbcMT2 gene (FIG. 4).

2.2 Transformation of Actinosynnema pretiosum Subsp. pretiosum

Escherichia coli ET12567, harbouring the plasmid pUZ8002 was transformed with pGP25 by electroporation to generate the E. coli donor strain for conjugation. This strain was used to transform Actinosynnema pretiosum subsp. pretiosum by vegetative conjugation (Matsushima et al., 1994). Exconjugants were plated on Medium 4 and incubated at 28° C. Plates were overlayed after 24 h with 50 mg/L apramycin and 25 mg/L nalidixic acid. As pGP25 is unable to replicate in Actinosynnema pretiosum subsp. pretiosum, any apramycin resistant colonies were anticipated to be transformants that contained plasmid integrated into the mbcMT2 gene of the chromosome by homologous recombination via the plasmid borne mbcMT2 internal fragment). This results in two truncated copies of the mbcMT2 gene on the chromosome. One exconjugant was isolated and tested for the production of macbecin analogues.

The colony was patched onto Medium 4 (with 50 mg/L apramycin and 25 mg/L nalidixic acid). A 6 mm circular plug from the patch was used to inoculate an individual 50 mL falcon tube containing 10 mL seed medium (variant of Medium 1-2% glucose, 3% soluble starch, 0.5% corn steep solids, 1% soybean flour, 0.5% peptone, 0.3% sodium chloride, 0.5% calcium carbonate) plus 50 mg/L apramycin. The seed culture was incubated for 2 days at 28° C., 200 rpm with a 5 cm throw. This was then used to inoculate (5% v/v) fermentation medium (Medium 2) and were grown at 28° C. for 24 hours and then at 26° C. for a further 5 days. Metabolites were extracted from the culture according to the standard protocol described above. Samples were assessed for production of macbecin analogues by HPLC using the standard protocol described in the general methods. The isolate produced novel compounds and was designated BIOT-3819.

2.3 Identification of Metabolites from BIOT-3819

LCMS was performed using Method 2 described above. No macbecin was observed and four new compounds were produced. These compounds all had benzoquinine chromophores and characteristics which led to the following structures being ascribed to them:

TABLE 4

| Compound | MS ions observed (m/z) |
|---|---|
| 15-O-desmethyl macbecin 14 | 543.4 [M − H]⁻; 567.5 [M + Na]⁺ |
| 11-O-desmethyl-15-O-desmethyl | 529.5 [M − H]⁻; 553.5 [M + Na]⁺ |
| macbecin 15 | |
| 11-O-desmethyl-15-O-desmethoxy | 513.5 [M − H]⁻; 537.5 [M + Na]⁺ |
| macbecin | |
| 15-desmethoxy macbecin | 527.5 [M − H]⁻; 551.5 [M + Na]⁺ |

2.4 Fermentation of BIOT-3819: an Actinosynnema Pretiosum Strain in which the O-methyltransferase mbcMT2 is Disrupted

Vegetative cultures were prepared by removing two agar plugs, 5 mm in diameter, from a MAM plate (Medium 4) and inoculating them into 30 mL medium 1 in 250 mL shake flasks containing 50 mg/L apramycin. The flasks were incubated at 28° C., 200 rpm (5 cm throw) for 48 h.

Vegetative cultures were inoculated at 5% v/v into 12×200 mL production medium (Medium 2) in 12×2 L shake flasks. Cultivation was carried out for 1 day at 28° C. followed by 5 days at 26° C., 200 rpm (5 cm throw).

2.5 Isolation of 15-O-desmethyl macbecin (14)

The fermentation broth (2.5 L) was extracted three times with an equal volume of ethyl acetate. The organic extracts were combined and the solvent removed in vacuo to yield 3.35 g of an oily residue. This was dissolved in methanol (15 mL) and a solution of FeCl₃ (1%, 200 mL) added. This was extracted with ethyl acetate (3×200 mL), the organic extracts combined and the solvent removed in vacuo to yield an oily residue (2.5 g). The residue was then chromatographed over Silica gel 60 eluting with a step gradient from CHCl₃:MeOH (99:1) to CHCl₃:methanol (95:5) and collecting fractions of ca. 200 mL. The fractions were analysed by LCMS (method 1) and four different sets of fractions, each containing one of the four different compounds. The fraction containing compound 14 was further purified by chromatography over Silica gel 60 eluting with EtOAc:Hexane (3:2) to give a white amorphous solid (292 mg).

### 2.6 Characterisation of 15-O-desmethylmacbecin (14)

TABLE 5

| | |
|---|---|
| | (14) |
| 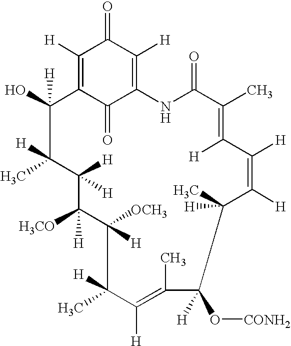 | |

| | ¹H NMR | | ¹³C NMR |
|---|---|---|---|
| Position | δ ppm | Multiplicity, Hz | δ ppm |
| 1 | — | — | 171.2 |
| 2 | — | — | 134.4 |
| 2-CH₃ | 1.94 | s | 13.4 |
| 3 | 7.24 | d, 12 | 131.2 |
| 4 | 6.36 | dd, 12, 11 | 125.7 |
| 5 | 5.67 | dd, 11, 7 | 143.5 |
| 6 | 3.18 | m | 35.4 |
| 6-CH₃ | 1.00 | d, 7.0 | 15.1 |
| 7 | 5.77 | d, 4.5 | 80.2 |
| 7-CONH₂ | — | — | 158.1 |
| 8 | — | — | 133.4 |
| 8-CH₃ | 1.58 | s | 16.1 |
| 9 | 5.33 | d, 9.5 | 130.1 |
| 10 | 2.50 | m | 39.5 |
| 10-CH₃ | 1.02 | d, 6.5 | 18.5 |
| 11 | 3.26 | dd, 2.5, 8.5 | 85.5 |
| 11-OCH₃ | 3.46 | s | 61.1 |
| 12 | 3.66 | ddd, 2.5, 3, 6 | 85.1 |
| 12-OCH₃ | 3.29 | s | 56.9 |
| 13 | 1.33 | m | 36.2 |
| 14 | 1.74 | m | 42.3 |
| 14-CH₃ | 0.80 | d, 7.0 | 14.7 |
| 15 | 5.14 | m | 68.8 |
| 16 | — | — | 150.8 |
| 17 | 6.65 | d, 1.5 | 134.6 |
| 18 | — | — | 189.5 |
| 19 | 7.12 | d, 1.5 | 113.4 |
| 20 | — | — | 141.1 |
| 21 | — | — | 185.0 |

### Example 3

### Generation of an Actinosynnema pretiosum strain carrying a deletion of the methyltransferase genes mbcMT1 and mbcMT2 and subsequently complemented by mbcMT1

3.1 Cloning of DNA Homologous to the Downstream Flanking Region of mbcMT2.

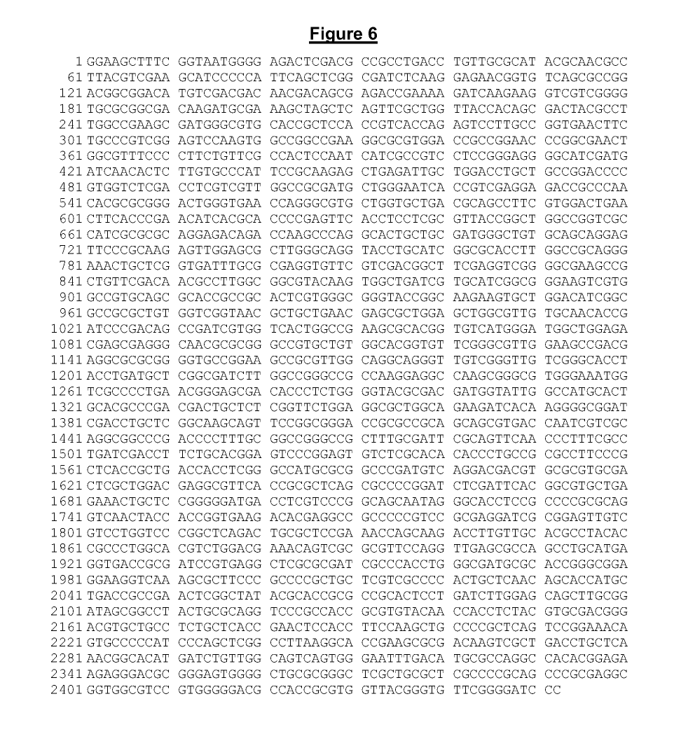

Oligos BioSG138 (SEQ ID NO: 15) and BioSG139 (SEQ ID NO: 16) were used to amplify a 2452 bp region of DNA from Actinosynnema pretiosum (ATCC 31280) using cosmid 52 (from example 1) as the template and standard PCR techniques. The HindIII and BamHI restriction sites introduced at the end of the primers are underlined. The amplified PCR product was cloned into vector Litmus28 previously linearised with EcoRV using standard techniques (FIG. 5). Plasmid Lit28PCR1 no6 was isolated and confirmed by DNA sequence analysis (SEQ ID NO: 17, FIG. 6). The analysis was completed using the sequencing primer BioSG150 (SEQ ID NO: 18).

BioSG138

(SEQ ID NO: 15)

5′-GGAAGCTTTCGGTAATGGGGAGACTCGACGCCGCCTGAC-3′
BioSG139

(SEQ ID NO: 16)

5′-GGGATCCCCGAACACCCGTAACCACGCGGTGGCGTCCCCC-3′
BioSG150

(SEQ ID NO: 18)

5′-CAGCAGGAGTTCCCGCAAGAGTTGGAGCGC-3′

3.2 Cloning of DNA Homologous to the Upstream Flanking Region of mbcMT1.

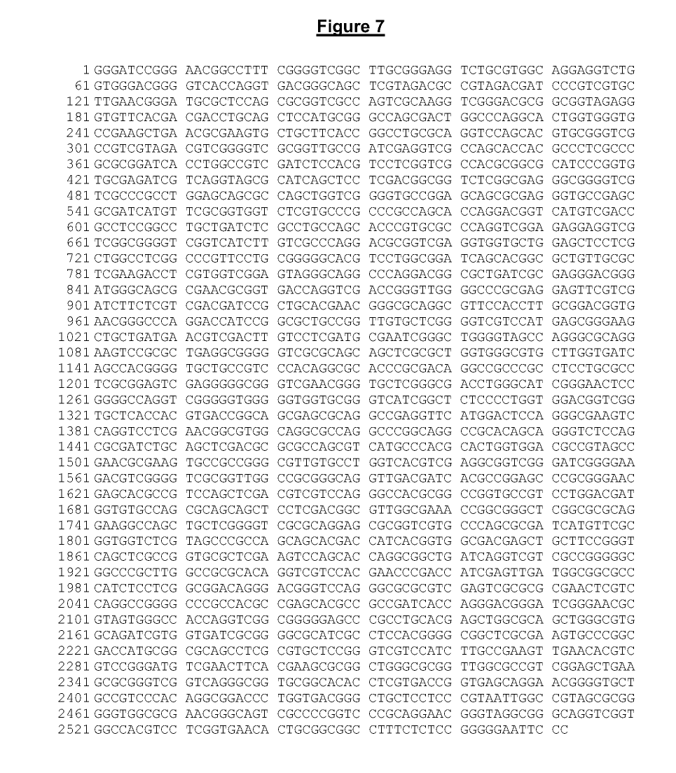

Oligos BioSG140 (SEQ ID NO: 19) and BioSG141 (SEQ ID NO: 20) were used to amplify a 2572 bp region of DNA from Actinosynnema pretiosum (ATCC 31280) using cosmid 52 (from example 1) as the template and standard PCR techniques. The BamHI and EcoRI restriction sites introduced at the end of the primers are underlined. The amplified PCR product was cloned into vector Litmus28 previously linearised with EcoRV using standard techniques (FIG. 5). Plasmid Lit28PCR2 no8 was isolated and confirmed by DNA sequence analysis (SEQ ID NO: 21 FIG. 7). The analysis was completed using the sequencing primer BioSG152 (SEQ ID NO: 22).

BioSG140

(SEQ ID NO: 19)

5′-GGGATCCGGGAACGGCCTTTCGGGGTCGGCTTGCGGGAGG-3′
BioSG141

(SEQ ID NO: 20)

5′-GGGAATTCCCCCGGAGAGAAAGGCCGCCGCAGTGTTCAC-3′
BioSG152

(SEQ ID NO: 22)

5′-CCTCGTGGTCGGAGTAGGGCAGGCCCAGGACGG-3′

3.3 Isolation of pKC1132PCR1

Plasmid Lit28PCR1 no6 was digested with HindIII/BamHI, the approximately 2.5 kb DNA insert was isolated and cloned into pKC1132 (Bierman et al., 1992) previously treated with HindIII/BamHI using standard techniques. Plasmid pKC1132PCR1 was isolated and confirmed by restriction digests (FIG. 5).

3.4 Isolation of pKC1132PCR1PCR2

Plasmid Lit28PCR2 no8 was digested with BamHI/EcoRI, the about 2.5 kb DNA insert was isolated and cloned into pKC1132PCR1 previously treated with BamHI/EcoRI using standard techniques. Plasmid pKC1132PCR1PCR2 was isolated and confirmed by restriction digests and sequence analysis.

3.5 Transformation of Actinosynnema pretiosum subsp. pretiosum

Escherichia coli ET12567, harbouring the plasmid pUZ8002 was used to transform pKC1132PCR1PCR2 by electroporation to generate the E. coli donor strain for conjugation. This strain was used for conjugation experiments in combination with Actinosynnema pretiosum subsp. pretiosum (Matsushima et al, 1994). Conjugated cells were plated on medium 4 (MAM medium) and incubated at 28° C. Plates were overlayed after 24 h with 50 mg/L apramycin and 25 mg/L nalidixic acid. Genomic DNA was isolated from apramycin resistant clones and plasmid integration was confirmed by Southern Blot analysis using standard techniques.

### 3.6 Screening for Secondary Recombinants

To isolate secondary recombinants clones were subjected to a series of subculturing steps in the absence of antibiotic selection followed by a protoplasting step to create colonies derived from single cells applying standard techniques. Four subculturing steps were employed using 20 mL ISP2 medium (Shirling and Gottlieb, 1966) in 250 ml conical flasks inoculated with 0.5 ml of culture and incubated as described in General Methods. 1 mL of glycine (10%) was added to the fourth subculturing step prior to protoplasting which was carried out using standard protocols. Colonies were patched in parallel onto MAM agar plates with and without the addition of apramycin and the plates were incubated at 28° C. for four days. Apramycin sensitive clones were re-patched to confirm the loss of the antibiotic marker. Deletion mutants were patched onto MAM medium and grown at 28° C. for four days. A 6 mm circular plug from each patch was used to inoculate individual 50 mL falcon tubes containing 10 mL of Medium 1 (seed medium). These seed cultures were incubated for 2 days at 28° C., 200 rpm with a 2 inch throw. These were then used to inoculate (0.5 mL into 10 mL) Medium 2 (production medium) and were grown at 28° C. for 24 hours and then at 26° C. for a further 5 days. Secondary metabolites were extracted from these cultures and samples were assessed for production of macbecin analogues by HPLC as described in General Methods.

3.7 Identification of Compounds from A. pretiosum ΔMT1 MT2 no13 (BIOT-3848)

LCMS analysis was performed using method 2. No macbecin was produced by this strain and production of 11-O-desmethyl-15-O-desmethylmacbecin, 15, was confirmed in the supernatant of clone A. pretiosum ΔMT1 MT2 no13 as expected. Chromatographic and MS analysis verified that it was identical to 11-O-desmethyl-15-O-desmethylmacbecin (15) that had been fully characterised elsewhere.

3.8 Isolation of Plasmid Lit28 mbcMT1

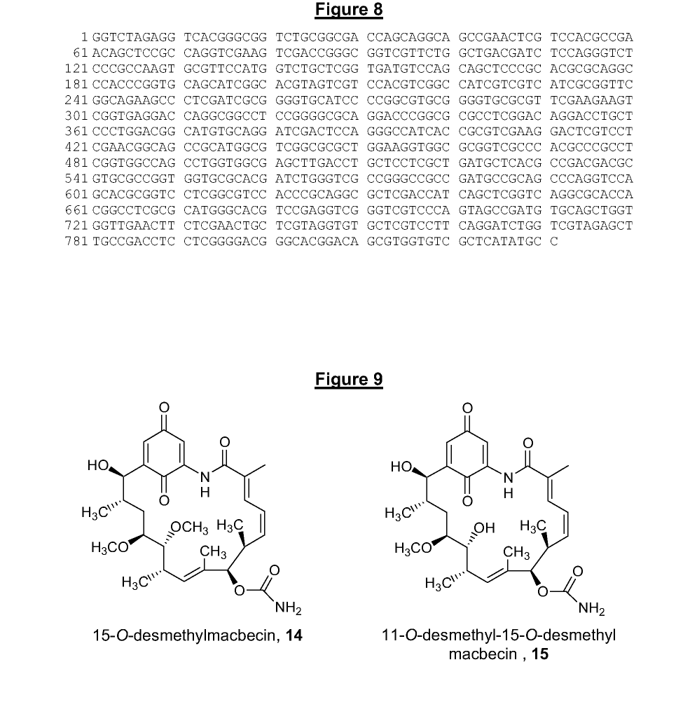

Oligos BioSG143 (SEQ ID NO: 23) and BioSG148 (SEQ ID NO: 24) were used to amplify of DNA from Actinosynnema pretiosum (ATCC 31280) using cosmid 52 (from example 1) as the template and standard PCR techniques. The XbaI and NdeI restriction sites introduced at the end of the primers are underlined. The amplified PCR product was cloned into vector Litmus28 previously linearised with EcoRV using standard techniques. Plasmid Lit28 mbcMT1 no15 was isolated and confirmed by DNA sequence analysis (SEQ ID NO: 25, FIG. 8).

BioSG143

(SEQ ID NO: 23)

5′-GGTCTAGAGGTCACGGGCGGTCTGCGGCGACCAGCAGG-3′
BioSG148

(SEQ ID NO: 24)

5′-GGCATATGAGCGACACCACGCTGTCCGTGCCCGTCCC-3′

3.9 Isolation of Plasmid pGP9 mbcMT1

Plasmid Lit28 mbcMT1 no15 was digested with NdeI/XbaI and the about 0.8 kb insert DNA fragment was isolated and cloned into NdeI/XbaI treated vector pGP9. Plasmid pGP9 mbcMT1 was isolated using standard techniques. The construct was confirmed by restriction digest analysis.

3.10 Complementation of A. pretiosum ΔMT1MT2 no13 with pGP9 mbcMT1

Conjugation experiments with Actinosynnema pretiosum subsp. pretiosum ΔMT1 MT2 no13 using plasmid pGP9 mbcMT1 were carried out as follows. Escherichia coli ET12567, harbouring the plasmid pUZ8002 was used to transform pGP9 mbcMT1 by electroporation to generate the E. coli donor strain for conjugation. This strain was used for conjugation experiments in combination with Actinosynnema pretiosum subsp. pretiosum (Matsushima et al, 1994). Exconjugants were plated on Medium 4 (MAM medium) and incubated at 28° C. Plates were overlayed after 24 h with 50 mg/L apramycin and 25 mg/L nalidixic acid.

Colonies were patched onto MAM agar plates with 50 mg/L apramycin and 25 mg/L nalidixic acid and the plates were incubated at 28° C. for four days. A 6 mm circular plug from each patch was used to inoculate individual 50 mL falcon tubes containing 10 mL of Medium 1 (seed medium) with 50 mg/L apramycin. These seed cultures were incubated for 2 days at 28° C., 200 rpm with a 2 inch throw. These were then used to inoculate (0.5 mL into 10 mL) Medium 2 (production medium) and were grown at 28° C. for 24 hours and then at 26° C. for a further 5 days. Secondary metabolites were extracted from these production cultures and samples were assessed for production of macbecin analogues by HPLC as described in General Methods.

The production of 15-O-desmethylmacbecin (14) in addition to 11-O-desmethyl-15-O-desmethylmacbecin (15) was confirmed. The isolated material was shown by chromatographic and MS methods to be identical to the 15-O-desmethylmacbecin (14) isolated and characterised in Example 2.

### Example 4

### Biological Data—In Vitro Evaluation of Anticancer Activity of Macbecin

In vitro evaluation of the test compounds for anticancer activity in a panel of human tumour cell lines in a monolayer proliferation assay was carried out as described in the general methods using a modified propidium iodide assay.

The results are displayed in Table 6 below; each result represents the mean of triplicate experiments, except for macbecin which represents the mean of duplicate experiments.

TABLE 6
in vitro cell line data

| | Test/Control (%) at drug concentration | | | |
|---|---|---|---|---|
| | Macbecin | | 14 | |
| Cell line | 1 (μg/mL) | 10 (μg/mL) | 1 (μg/mL) | 10 (μg/mL) |
| CNXF 498NL | 29 | 14 | 49.3 | 4.7 |
| CXF HT29 | 22 | 8 | 58.3 | 7.7 |
| LXF 1121L | 30 | 17 | 63 | 6 |
| MCF-7 | 42 | 19 | 65.3 | 15.3 |
| MEXF 394NL | 16 | 13 | 17 | 2.3 |
| DU145 | 13 | 5 | 49.7 | 6 |

All references including patent and patent applications referred to in this application are incorporated herein by reference to the fullest extent possible.
Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

### REFERENCES

Allen, I. W. and Ritchie, D. A. (1994) Cloning and analysis of DNA sequences from Streptomyces hygroscopicus encoding geldanamycin biosynthesis. Mol. Gen. Genet. 243: 593-599.
Bagatell, R. and Whitesell, L. (2004) Altered Hsp90 function in cancer: A unique therapeutic opportunity. Molecular Cancer Therapeutics 3: 1021-1030.
Beliakoff, J. and Whitesell, L. (2004) Hsp90: an emerging target for breast cancer therapy. Anti-Cancer Drugs 15:651-662.
Bierman, M., Logan, R., O'Brien, K., Seno, E T., Nagaraja Rao, R. and Schoner, B E. (1992) “Plasmid cloning vectors for the conjugal transfer of DNA from Escherichia coli to Streptomyces spp.” Gene 116: 43-49.
Blagosklonny, M. V. (2002) Hsp-90-associated oncoproteins: multiple targets of geldanamycin and its analogues. Leukemia 16:455-462.
Blagosklonny, M. V., Toretsky, J., Bohen, S, and Neckers, L. (1996) Mutant conformation of p53 translated in vitro or in vivo requires functional HSP90. Proc. Natl. Acad. Sci. USA 93:8379-8383.
Bohen, S. P. (1998) Genetic and biochemical analysis of p23 and ansamycin antibiotics in the function of Hsp90-dependent signaling proteins. Mol Cell Biol 18:3330-3339.
Carreras, C. W., Schirmer, A., Zhong, Z. and Santi D. V. (2003) Filter binding assay for the geldanamycin-heat shock protein 90 interaction. Analytical Biochemistry 317:40-46.
Cassady, J. M., Chan, K. K., Floss, H. G. and Leistner E. (2004) Recent developments in the maytansinoid antitumour agents. Chem. Pharm. Bull. 52(1) 1-26.
Chiosis, G., Huezo, H., Rosen, N., Mimnaugh, E., Whitesell, J. and Neckers, L. (2003) 17AAG: Low target binding affinity and potent cell activity—finding an explanation. Molecular Cancer Therapeutics 2:123-129.
Chiosis, G., Vilenchik, M., Kim, J. and Solit, D. (2004) Hsp90: the vulnerable chaperone. Drug Discovery Today 9:881-888.
Csermely, P. and Soti, C. (2003) Inhibition of Hsp90 as a special way to inhibit protein kinases. Cell. Mol. Biol. Lett. 8:514-515.
DeBoer, C. and Dietz, A. (1976) The description and antibiotic production of Streptomyces hygroscopicus var. geldanus. J. Antibiot. 29:1182-1188.
DeBoer, C., Meulman, P. A., Wnuk, R. J., and Peterson, D. H. (1970) Geldanamycin, a new antibiotic. J. Antibiot. 23:442-447.
Dengler W. A., Schulte J., Berger D. P., Mertelsmann R. and Fiebig H H. (1995) Development of a propidium iodide fluorescence assay for proliferation and cytotoxicity assay. Anti-Cancer Drugs, 6:522-532.
Dikalov, s., Landmesser, U., Harrison, D G., 2002, Geldanamycin Leads to Superoxide Formation by Enzymatic and Non-enzymatic Redox Cycling, The Journal of Biological Chemistry, 277(28), pp 25480-25485
Donzé O. and Picard, D. (1999) Hsp90 binds and regulates the ligand-inducible α subunit of eukaryotic translation initiation factor kinase Gcn2. Mol Cell Biol 19:8422-8432.
Egorin M J, Lagattuta T F, Hamburger D R, Covey J M, White K D, Musser S M, Eiseman J L. (2002) “Pharmacokinetics, tissue distribution, and metabolism of 17-(dimethylaminoethylamino)-17-demethoxygeldanamycin (NSC 707545) in CD2F1 mice and Fischer 344 rats.” Cancer Chemother Pharmacol, 49(1), pp 7-19.
Eustace, B. K., Sakurai, T., Stewart, J. K., et al. (2004) Functional proteomic screens reveal an essential extracellular role for hsp90α in cancer cell invasiveness. Nature Cell Biology 6:507-514.
Fang, Y., Fliss, A. E., Rao, J. and Caplan A. J. (1998) SBA1 encodes a yeast Hsp90 cochaperone that is homologous to vertebrate p23 proteins. Mol Cell Biol 18:3727-3734.
Fiebig H. H., Dengler W. A. and Roth T. Human tumor xenografts: Predictivity, characterization, and discovery of new anticancer agents. In: Fiebig H H, Burger A M (eds). Relevance of Tumor Models for Anticancer Drug Development. Contrib. Oncol. 1999, 54: 29-50.
Goetz, M. P., Toft, D. O., Ames, M. M. and Ehrlich, C. (2003) The Hsp90 chaperone complex as a novel target for cancer therapy. Annals of Oncology 14:1169-1176.
Harris, S. F., Shiau A. K. and Agard D. A. (2004) The crystal structure of the carboxy-terminal dimerization domain of htpG, the Escherichia coli Hsp90, reveals a potential substrate binging site. Structure 12: 1087-1097.
Hong, Y.-S., Lee, D., Kim, W., Jeong, J.-K. et al. (2004) Inactivation of the carbamoyltransferase gene refines post-polyketide synthase modification steps in the biosynthesis of the antitumor agent geldanamycin. J. Am. Chem. Soc. 126:11142-11143.
Hostein, I., Robertson, D., DiStefano, F., Workman, P. and Clarke, P. A. (2001) Inhibition of signal transduction by the Hsp90 inhibitor 17-allylamino-17-demethoxygeldanamycin results in cytostasis and apoptosis. Cancer Research 61:4003-4009.
Hu, Z., Liu, Y., Tian, Z.-Q., Ma, W., Starks, C. M. et al. (2004) Isolation and characterization of novel geldanamycin analogues. J. Antibiot. 57:421-428.
Hur, E., Kim, H.-H., Choi, S. M., et al. (2002) Reduction of hypoxia-induced transcription through the repression of hypoxia-inducible factor-1α/aryl hydrocarbon receptor nuclear translocator DNA binding by the 90-kDa heat-shock protein inhibitor radicicol. Molecular Pharmacology 62:975-982.
Iwai Y, Nakagawa, A., Sadakane, N., Omura, S., Oiwa, H., Matsumoto, S., Takahashi, M., Ikai, T., Ochiai, Y. (1980) Herbimycin B, a new benzoquinoid ansamycin with anti-TMV and herbicidal activities. The Journal of Antibiotics, 33(10), pp1114-1119.
Jez, J. M., Chen, J. C.-H., Rastelli, G., Stroud, R. M. and Santi, D. V. (2003) Crystal structure and molecular modeling of 17-DMAG in complex with human Hsp90. Chemistry and Biology 10:361-368.
Kaur, G., Belotti, D, Burger, A. M., Fisher-Nielson, K., Borsotti, P. et al. (2004) Antiangiogenic properties of 17-(Dimethylaminoethylamino)-17-Demethoxygeldanamycin: an orally bioavailable heat shock protein 90 modulator. Clinical Cancer Research 10:4813-4821.
Kieser, T., Bibb, M. J., Buttner, M. J., Chater, K. F., and Hopwood, D. A. (2000) Practical Streptomyces Genetics, John Innes Foundation, Norwich
Kumar, R., Musiyenko, A. and Barik S. (2003) The heat shock protein 90 of Plasmodium falciparum and antimalarial activity of its inhibitor, geldanamycin. J Malar 2:30.
Kurebayashi, J., Otsuke, T., Kurosumi, M., Soga, S., Akinaga, S, and Sonoo, H. (2001) A radicicol derivative, KF58333, inhibits expression of hypoxia-inducible factor-1α and vascular endothelial growth factor, angiogenesis and growth of human breast cancer xenografts. Jpn. J. Cancer Res. 92:1342-1351.
Le Brazidec, J.-Y., Kamal, A., Busch, D., Thao, L., Zhang, L. et al. (2003) Synthesis and biological evaluation of a new class of geldanamycin derivatives as potent inhibitors of Hsp90. J. Med. Chem. 47: 3865-3873.
Lee, Y.-S., Marcu, M. G. and Neckers, L. (2004) Quantum chemical calculations and mutational analysis suggest heat shock protein 90 catalyzes trans-cis isomeration of geldanamycin. Chem. Biol. 11:991-998.
Liu, X.-D., Morano, K. A. and Thiele D. J. (1999); The yeast Hsp10 family member, Sse1, is an Hsp90 cochaperone. J Biol Chem 274:26654-26660.
Mandler, R., Wu, C., Sausville, E. A., Roettinger, A. J., Newman, D. J., Ho, D. K., King, R., Yang, D., Lippman, M. E., Landolfi, N. F., Dadachova, E., Brechbiel, M. W. and Waldman, T. A. (2000) Immunoconjugates of geldanamycin and anti-HER2 monoclonal antibodies: antiproliferative activity on human breast carcinoma cell lines. Journal of the National Cancer Institute 92:1573-1581.
Matsushima, P., M. C. Broughton, et al. (1994). Conjugal transfer of cosmid DNA from Escherichia coli to Saccharopolyspora spinosa: effects of chromosomal insertions on macrolide A83543 production. Gene 146(1): 39-45.
McLaughlin S. H., Smith, H. W. and Jackson S. E. (2002) Stimulation of the weak ATPase activity of human Hsp90 by a client protein. J. Mol. Biol. 315: 787-798.
McCammon, M. T. and L. W. Parks (1981). Inhibition of sterol transmethylation by S-adenosylhomocysteine analogs. J Bacteriol 145(1): 106-12.
Muroi M, Izawa M, Kosai Y, Asai M. (1981) “The structures of macbecin I and II” Tetrahedron, 37, pp 1123-1130.
Muroi, M., Izawa M., Kosai, Y., and Asai, M. (1980) Macbecins I and II, New Antitumor antibiotics. II. Isolation and characterization. J Antibiotics 33:205-212.
Neckers, L (2003) Development of small molecule Hsp90 inhibitors: utilizing both forward and reverse chemical genomics for drug identification. Current Medicinal Chemistry 9:733-739.
Neckers, L. (2002) Hsp90 inhibitors as novel cancer chemotherapeutic agents. Trends in Molecular Medicine 8:S55-S61.
Nimmanapalli, R., O'Bryan, E., Kuhn, D., Yamaguchi, H., Wang, H.-G. and Bhalla, K. N. (2003) Regulation of 17-AAG-induced apoptosis: role of Bcl-2, Bcl-x_{L}, and Bax downstream of 17-AAG-mediated down-regulation of Akt, Raf-1, and Src kinases. Neoplasia 102:269-275.
Omura, S., Iwai, Y., Takahashi, Y., Sadakane, N., Nakagawa, A., Oiwa, H., Hasegawa, Y., Ikai, T., (1979), Herbimycin, a new antibiotic produced by a strain of Streptomyces. The Journal of Antibiotics, 32(4), pp 255-261.
Omura, S., Miyano, K., Nakagawa, A., Sano, H., Komiyama, K., Umezawa, I., Shibata, K, Satsumabayashi, S., (1984), “Chemical modification and antitumor activity of Herbimycin A. 8,9-epoxide, 7,9-carbamate, and 17 or 19-amino derivatives”. The Journal of Antibiotics, 37(10), pp 1264-1267.
Ono, Y., Kozai, Y. and Ootsu, K. (1982) Antitumor and cytocidal activities of a newly isolated benzenoid ansamycin, Macbecin I. Gann. 73:938-44.
Patel, K., M. Piagentini, Rascher, A., Tian, Z. Q., Buchanan, G. O., Regentin, R., Hu, Z., Hutchinson, C. R. And McDaniel, R. (2004). “Engineered biosynthesis of geldanamycin analogs for hsp90 inhibition.” Chem Biol 11 (12): 1625-33.
Pfeifer, B. A. and C. Khosla (2001). “Biosynthesis of polyketides in heterologous hosts.” Microbiology and Molecular Biology Reviews 65(1): 106-118.
Rascher, A., Hu, Z., Viswanathan, N., Schirmer, A. et al. (2003) Cloning and characterization of a gene cluster for geldanamycin production in Streptomyces hygroscopicus NRRL 3602. FEMS Microbiology Letters 218:223-230.
Rascher, A., Z. Hu, Buchanan, G. O., Reid, R. and Hutchinson, C. R. (2005). Insights into the biosynthesis of the benzoquinone ansamycins geldanamycin and herbimycin, obtained by gene sequencing and disruption. Appl Environ Microbiol 71(8): 4862-71.
Rawlings, B. J. (2001). “Type I polyketide biosynthesis in bacteria (Part B).” Natural Product Reports 18(3): 231-281.
Roth T., Burger A. M., Dengler W., Willmann H. and Fiebig H. H. Human tumor cell lines demonstrating the characteristics of patient tumors as useful models for anticancer drug screening. In: Fiebig H H, Burger A M (eds). Relevance of Tumor Models for Anticancer Drug Development. Contrib. Oncol. 1999, 54: 145-156.
Rowlands, M. G., Newbatt, Y. M., Prodromou, C., Pearl, L. H., Workman, P. and Aherne, W. (2004) High-throughput screening assay for inhibitors of heat-shock protein 90 ATPase activity. Analytical Biochemistry 327:176-183
Schulte, T. W., Akinaga, S., Murakata, T., Agatsuma, T. et al. (1999) Interaction of radicicol with members of the heat shock protein 90 family of molecular chaperones. Molecular Endocrinology 13:1435-1488.
Shibata, K., Satsumabayashi, S., Nakagawa, A., Omura, S. (1986a) The structure and cytocidal activity of herbimycin C. The Journal of Antibiotics, 39(11), pp 1630-1633.
Shibata, K., Satsumabayashi, S., Sano, H., Komiyama, K., Nakagawa, A., Omura, S. (1986b) Chemical modification of Herbimycin A: synthesis and in vivo antitumor activities of halogenated and other related derivatives of herbimycin A. The Journal of Antibiotics, 39(3), pp 415-423.
Shirling, E. B. and Gottlieb, D. (1966) International Journal of Systematic Bacteriology 16:313-340
Smith-Jones, P. M., Solit, D. B., Akhurst, T., Afroze, F., Rosen, N. and Larson, S. M. (2004) Imaging the pharmacodynamics of HER2 degradation in response to Hsp90 inhibitors. Nature Biotechnology 22:701-706.
Spiteller, P., Bai, L., Shang, G., Carroll, B. J., Yu, T.-W. and Floss, H. G. (2003). The post-polyketide synthase modification steps in the biosynthesis of the antitumor agent ansamitocin by Actinosynnema pretiosum. J Am Chem Soc 125(47): 14236-7
Sreedhar A. S., Nardai, G. and Csermely, P. (2004) Enhancement of complement-induced cell lysis: a novel mechanism for the anticancer effects of Hsp90 inhibitors. Immunology letters 92:157-161.
Sreedhar, A. S., Söti, C. and Csermely, P. (2004a) Inhibition of Hsp90: a new strategy for inhibiting protein kinases. Biochimica Biophysica Acta 1697:233-242.
Staunton, J. and K. J. Weissman (2001). “Polyketide biosynthesis: a millennium review.” Natural Product Reports 18(4): 380-416.
Stead, P., Latif, S., Blackaby, A. P. et al. (2000) Discovery of novel ansamycins possessing potent inhibitory activity in a cell-based oncostatin M signalling assay. J Antibiotics 53:657-663.
Supko, J. G., Hickman, R. L., Grever, M. R. and Malspeis, L (1995) Preclinical pharmacologic evaluation of geldanamycin as an antitumor agent. Cancer Chemother. Pharmacol. 36:305-315.
Takahashi, A., Casais, C., Ichimura K. and Shirasu, K. (2003) HSP90 interacts with RAR1 and SGT1 and is essential for RPS2-mediated disease resistance in Arabidopsis. Proc. Natl. Acad. Sci. USA 20:11777-11782.
Tanida, S., Hasegawa, T. and Higashide E. (1980) Macbecins I and II, New Antitumor antibiotics. I. Producing organism, fermentation and antimicrobial activities. J Antibiotics 33:199-204.
Tian, Z.-Q., Liu, Y., Zhang, D., Wang, Z. et al. (2004) Synthesis and biological activities of novel 17-aminogeldanamycin derivatives. Bioorganic and Medicinal Chemistry 12:5317-5329.
Uehara, Y. (2003) Natural product origins of Hsp90 inhibitors. Current Cancer Drug Targets 3:325-330.
Vasilevskaya, I. A., Rakitina, T. V. and O'Dwyer, P. J. (2003) Geldanamycin and its 17-Allylamino-17-Demethoxy analogue antagonize the action of cisplatin in human colon adenocarcinoma cells: differential caspase activation as a basis of interaction. Cancer Research 63: 3241-3246.
Watanabe, K., Okuda, T., Yokose, K., Furumai, T. and Maruyama, H. H. (1982) Actinosynnema mirum, a new producer of nocardicin antibiotics. J. Antibiot. 3:321-324.
Wegele, H., Müller, L. and Buchner, J. (2004) Hsp70 and Hsp90-a relay team for protein folding. Rev Physiol Biochem Pharmacol 151:1-44.
Wenzel, S. C., Gross, F, Zhang, Y., Fu, J., Stewart, A. F. and Müller, R (2005) Heterologous expression of a myxobacterial natural products assembly line in Pseudomonads via Red/ET recombineering. Chemistry & Biology 12: 249-356.
Whitesell, L., Mimnaugh, E. G., De Costa, B., Myers, C. E. and Neckers, L. M. (1994) Inhibition of heat shock protein HSP90-pp 60^{v-src} heteroprotein complex formation by benzoquinone ansamycins: Essential role for stress proteins in oncogenic transformation. Proc. Natl. Acad. Sci. USA 91: 8324-8328.
Winklhofer, K. F., Heller, U., Reintjes, A. and Tatzelt J. (2003) Inhibition of complex glycosylation increases the formation of PrP^{sc}. Traffic 4:313-322.
Workman P. (2003) Auditing the pharmacological accounts for Hsp90 molecular chaperone inhibitors: unfolding the relationship between pharmacokinetics and pharmacodynamics. Molecular Cancer Therapeutics 2:131-138.
Workman, P. and Kaye, S. B. (2002) Translating basic cancer research into new cancer therapeutics. Trends in Molecular Medicine 8:S1-S9.
Young, J. C.; Moarefi, I. and Hartl, U. (2001) Hsp90: a specialized but essential protein folding tool. J. Cell. Biol. 154:267-273.

1. A 15-O-desmethylmacbecin analogue according to the formula (IA) or (IB) below, or a pharmaceutically acceptable salt thereof: 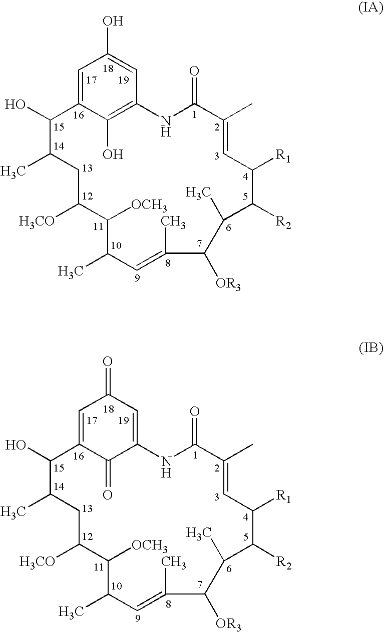
wherein:
R₁ and R₂ either both represent H or together they represent a bond (i.e. C4 to C5 is a double bond); and
R₃=H or CONH₂.

2. The compound according to claim 1, wherein the 15-O-desmethylmacbecin analogue is according to formula (IA).

3. The compound according to claim 1, wherein the 15-O-desmethylmacbecin analogue is according to formula (IB).

4. The compound according to claim 1, wherein R₃ represents CONH₂.

5. The compound according to claim 1, wherein R₁ and R₂ together represent a bond.

6. The compound according to claim 1, wherein R₁ and R₂ together represent a bond and R₃ represents CONH₂.

7. The 15-O-desmethylmacbecin analogue according to claim 1 which is: 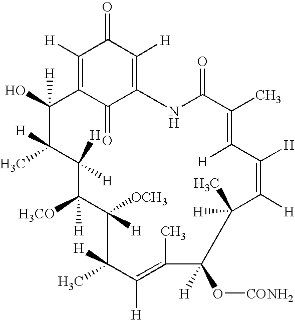
or a pharmaceutically acceptable salt thereof.

8. A pharmaceutical composition comprising a 15-O-desmethylmacbecin analogue according to claim 1, together with one or more pharmaceutically acceptable diluents or carriers.

9-11. (canceled)

12. A method of treatment of cancer, B-cell malignancies, malaria, fungal infection, diseases of the central nervous system and neurodegenerative diseases, diseases dependent on angiogenesis, autoimmune diseases and/or as a prophylactic pretreatment for cancer which comprises administering to a patient in need thereof an effective amount of an 15-O-desmethylmacbecin analogue according to claim 1.

13. The method according to claim 12, wherein the 15-O-desmethylmacbecin analogue is administered in combination with another treatment.

14. The method according to claim 13 where the other treatment is selected from the group consisting of: methotrexate, leukovorin, adriamycin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody, capecitabine, raloxifene hydrochloride, EGFR inhibitors, VEGF inhibitors, proteasome inhibitors radiotherapy and surgery.

15. The method according to claim 13 where the other treatment is selected from the group consisting of conventional chemotherapeutics such as cisplatin, cytarabine, cyclohexylchloroethylnitrosurea, cyclophosphamide, gemcitabine, Ifosfamid, leucovorin, mitomycin, mitoxantone, oxaliplatin and taxanes including taxol and vindesine; hormonal therapies such as anastrozole, goserelin, megestrol acetate and prenisone; monoclonal antibody therapies such as cetuximab (anti-EGFR); protein kinase inhibitors such as dasatinib, lapatinib; histone deacetylase (HDAC) inhibitors such as vorinostat; angiogenesis inhibitors such as sunitinib, sorafenib, lenalidomide; and mTOR inhibitors such as temsirolimus.

16. A method for the production of a 15-O-desmethylmacbecin analogue according to claim 1, said method comprising:
a) providing a first host strain that produces macbecin when cultured under appropriate conditions;
b) deleting or inactivating one or more post-PKS genes, wherein at least one of the post-PKS genes is mbcMT2, or a homologue thereof;
c) culturing said modified host strain under suitable conditions for the production of novel compounds; and
d) optionally isolating the compounds produced.

17. A method for the production of an 15-O-desmethylmacbecin analogue according to claim 1, said method comprising:
a) providing a first host strain that produces macbecin or an analogue thereof when cultured under appropriate conditions;
b) deleting or inactivating one or more post-PKS genes, wherein at least one of the post-PKS genes is mbcMT2, or a homologue thereof;
c) re-introducing some or all of the post-PKS genes not including mbcMT2, or a homologue thereof;
d) culturing said modified host strain under suitable conditions for the production of 15-O-desmethylmacbecin analogues; and
e) optionally isolating the compounds produced.

18. A host strain which naturally produces macbecin and analogues thereof, in which the mbcMT2 gene or a homologue thereof has been deleted or inactivated such that it thereby produces 15-O-desmethylmacbecin or an analogue thereof.

19. An engineered strain based on a macbecin producing strain in which mbcMT2 and optionally further post-PKS genes have been deleted or inactivated.

20. The strain according to claim 19 in which mbcMT1 and mbcMT2 have been deleted or inactivated and mbcMT1 has been reintroduced.

21. The strain according to claim 18 which is A. pretiosum or A. mirum.

22. A process for producing 15-O-desmethylmacbecin or an analogue thereof which comprises culturing a strain according to claim 18.

23. The process according to claim 22 further comprising the step of isolating 15-β-desmethylmacbecin or an analogue thereof.

24. (canceled)

25. (canceled)

26. The strain according to claim 19 which is A. pretiosum or A. mirum.

27. A process for producing 15-O-desmethylmacbecin or an analogue thereof which comprises culturing a strain according to claim 19.

28. The process according to claim 27 further comprising the step of isolating 15-O-desmethylmacbecin or an analogue thereof.

29. The composition according to claim 8 further comprising another treatment.

30. The composition according to claim 29 where the other treatment is selected from the group consisting of: methotrexate, leukovorin, adriamycin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody, capecitabine, raloxifene hydrochloride, EGFR inhibitors, VEGF inhibitors, proteasome inhibitors radiotherapy and surgery.

31. The composition according to claim 29 where the other treatment is selected from the group consisting of conventional chemotherapeutics such as cisplatin, cytarabine, cyclohexylchloroethylnitrosurea, cyclophosphamide, gemcitabine, Ifosfamid, leucovorin, mitomycin, mitoxantone, oxaliplatin and taxanes including taxol and vindesine; hormonal therapies such as anastrozole, goserelin, megestrol acetate and prenisone; monoclonal antibody therapies such as cetuximab (anti-EGFR); protein kinase inhibitors such as dasatinib, lapatinib; histone deacetylase (HDAC) inhibitors such as vorinostat; angiogenesis inhibitors such as sunitinib, sorafenib, lenalidomide; and mTOR inhibitors such as temsirolimus.
